SEQUENCE LISTING (20 sequences)

SEQ ID NO: 1 - protein, 1057 aa, synthetic construct
QLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRA

SEQ ID NO: 2 - protein, 1040 aa, synthetic construct
QYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRA

SEQ ID NO: 3 - DNA, 3174 bp, synthetic construct
  source 1..3174: mol_type = other DNA
cagttgtcttatggttatgatgaaaaatcaactggaggaattagtgttccaggtccaatgggaccatctggaccaagaggtcttcctggacctccaggtgctcctggtccacagggttttcagggaccaccaggagaaccaggagagccaggagcttcaggtcctatgggtccaagaggtccacctggccctccaggaaagaatggtgatgatggagaagcaggaaagcctggtcgtccaggcgaaagaggtcctcctggaccacaaggggctagaggactgcctggtactgctggacttccaggaatgaaaggtcatagaggtttttctggacttgacggtgctaagggagatgcaggaccagctggacctaagggtgagccaggatctccaggcgagaacggagcccctggtcagatgggaccaagaggattgccaggtgaaagaggaaggcctggagctcctggtccagctggagctaggggtaatgatggagctactggagctgcaggacctcctggtccaactggtcctgctggaccaccaggttttcctggagctgtgggagctaaaggtgaggctggtccacaaggtcctagaggatcagaaggaccccaaggagttagaggagaaccaggtccacctggaccagccggtgcagctggtcctgctggtaatcctggtgctgacggacaacctggcgctaaaggtgcaaacggagctcctggaatcgcaggtgctccaggttttccaggtgcaagaggtcctagtggtccacagggtccaggaggtccaccaggaccaaagggtaatagtggtgagcctggagctccaggaagcaaaggagatactggtgctaagggcgaaccaggaccagttggagtgcaaggacctccaggaccagcaggagaagaaggtaagagaggagctaggggagaaccaggacctactggtttgccaggaccaccaggtgaacgtggaggacctggatcaaggggttttccaggagctgatggggttgctggtcctaagggaccagcaggagaaagaggatctccaggtcctgctggaccaaaaggaagtcctggagaagctggcagacctggagaagcaggtcttccaggtgctaagggtcttactggatctccaggatctcctggtcctgatggaaaaactggaccaccaggtcctgctggacaagacggtagacctggacctcctggtccacctggagctagaggtcaagctggtgttatgggatttcctggaccaaagggtgctgctggtgaaccagggaaagctggtgaaagaggagtgcctggtccacctggagctgttggccctgctggaaaggatggtgaagctggtgctcaaggaccaccaggacctgctggtcctgctggagaaagaggtgagcagggaccagctggaagtcctggttttcaaggattgccaggaccagctggtcctccaggggaagcaggtaagccaggcgaacaaggagtcccaggagatttgggagctcctggaccatctggagcaagaggtgaaagaggatttccaggagaaagaggagttcagggtccgccaggacctgctggaccaagaggagcaaacggagcaccaggaaatgatggagctaagggggatgctggtgctccaggtgcacctggatctcaaggagctccaggactccaaggaatgcctggtgaaagaggtgctgctggtcttccaggacctaagggagatagaggagatgcaggaccaaagggagctgatggaagccctggtaaggatggtgttagaggacttactggaccaataggtcctcctggtccagctggagcacctggggataagggtgagagtggtccttctggtcctgcaggcccaacaggagcaagaggtgctcctggtgatagaggtgaacctggacctccaggtcctgctggatttgctggtccacctggtgctgatggacaacctggagcaaagggagagcctggagatgcaggagcaaaaggagatgctggtccacctggaccagctggtcctgctggtcctcctggaccaatcggtaatgttggagctcctggtgctaaaggtgctaggggttcagctggacctcctggagctactggttttcctggtgctgctggcagggttggaccacctggtccaagtggaaatgccggaccacctggcccaccaggaccagctggaaaagaaggtggaaaaggaccaagaggagaaactggtccagcaggtcgtccaggtgaagtgggccctccaggcccaccaggacctgctggagaaaagggaagtccaggtgcagatggaccagctggcgctcctggtactccaggacctcagggtatcgctggacaaagaggtgttgttggtttgccaggtcagagaggagagagaggttttccaggattgccaggtccttctggtgagcctggtaaacagggtccttctggagcttctggtgaaagaggacctcctggtcctatgggtccaccaggattggcaggaccaccaggtgaatctggaagagaaggtgcaccaggagcagaaggatctccaggtagggatggaagccctggggctaaaggagataggggagaaactggaccagcaggaccaccaggtgctcctggtgccccaggtgctcctggaccagttggtcctgctggtaagtctggtgacagaggtgaaactgggccagctggaccagctggacctgttggtcctgttggtgctagaggtccagctggacctcaaggtcctagaggagataaaggagaaactggtgaacaaggtgataggggtattaagggtcataggggattttctggtttgcaaggaccacctggaccaccaggttcaccaggagagcaaggtccaagtggagcatctggaccagctggtccaaggggacctcctggatctgctggagctccaggtaaagatggacttaatggtcttccaggtccaattggacctcctggaccaagaggaagaactggagatgcaggaccagttggaccaccaggtccaccaggacctcctggtcctccaggacctccaagtgcaggttttgatttttcatttcttcctcaaccaccacaagagaaggctcacgatggaggaaggtattatagagcttaa

SEQ ID NO: 4 - DNA, 3123 bp, synthetic construct
  source 1..3123: mol_type = other DNA
caatacgatggaaaaggagttggtctcggaccaggtccaatgggtttgatgggaccaagaggtcctccaggagctgctggtgctcctggtcctcaaggatttcaaggaccagctggagagccaggtgagcctggacagactggtccagctggtgcaagaggacctgcaggacctcccggtaaagctggtgaagatggacatccaggaaaaccaggaagaccaggagagaggggtgtcgttggaccacaaggtgcaagaggttttccaggaacaccaggtcttcctggttttaaaggtattagagggcacaatggtttggatggtttgaagggtcaaccaggtgctccaggagttaaaggagaacctggtgctccaggtgaaaatggtactccgggacaaactggagcaagaggattgcctggagaaaggggtcgtgttggtgcaccaggtcctgcaggagcaagaggttcagatggatctgtgggtccagttggtcctgctggaccaattggttctgctggtcctcctggatttccaggagctcctggaccaaagggagaaattggagcagttggaaatgcaggacctgctggtcctgctggacctagaggtgaagttggattgcctggtttgtcgggcccagtaggtcctccaggaaatccaggagctaatggattgactggtgctaaaggagctgctggattgcctggtgtggcaggtgctcctggtcttccaggtcctagaggcattcctggtccagtaggagctgcaggagctactggtgcaagaggtcttgttggagaaccaggacccgcaggttcaaaaggagaatctggaaataaaggtgaaccaggatctgctggacctcagggtccacctggtcctagtggtgaagaaggaaagagaggacctaatggtgaggccggaagcgctggtcctcctggaccaccaggtcttagaggaagtcctggtagtagaggattgccaggagcagatggaagagctggtgttatgggaccaccaggttctagaggagctagcggaccagctggagtgaggggtccaaatggagatgctggaaggcctggagaaccaggattgatgggtcctaggggtttaccaggaagtccaggaaatattggaccagcaggtaaagaaggacctgtgggtttgccaggaattgatggaaggccaggaccaattggaccagctggtgctagaggagagcctggtaatattggttttccaggtccaaagggtccaactggagaccctggaaagaacggtgataaaggacatgcaggacttgctggagcaagaggagctcctggccctgatggtaataatggtgctcaaggtcctccaggaccacaaggtgttcaaggaggaaaaggtgagcaaggaccacctggaccaccaggttttcaaggacttcctggcccatctggtccagctggtgaagttggaaaaccaggagagagaggtcttcatggagaatttggacttccaggaccagctggccctagaggagaaagaggacctccaggtgaatctggtgctgcaggtccaactggaccaattggttccagaggaccatccggacctcctggaccagatggaaataaaggtgaaccaggagttgtgggtgctgttggtacagcaggtccatcaggtccatctggtcttccaggagagaggggcgctgctggtattcctggtggaaagggagagaagggcgaaccaggactcagaggtgaaattggaaatcccggaagagatggagcaagaggagctcctggagctgttggtgctccaggaccagctggtgcaacaggtgataggggtgaagctggggctgctggacctgctggaccagctggtcctaggggttctcctggagaaagaggtgaggtaggtcctgctggacctaatggttttgctgggccagccggtgctgctggacaaccaggagccaagggagagagaggagctaaaggaccaaaaggagagaatggagtcgttggtcctactggaccagttggagctgctggaccagctggaccaaatggaccaccaggaccagctggatctagaggagatggtggaccaccaggtatgacaggtttcccaggtgcagctggaaggactggacctccagggccatcaggtatttctggacctccaggaccaccaggtccagctggaaaagagggtctcagaggaccaagaggagatcaaggaccagtgggaagaacaggtgaagtgggtgctgtgggtccacctggttttgctggggagaaaggtccttccggagaagctggaactgcaggtccaccaggaactccaggtccacagggtttgcttggagctcctggaattcttggtttacctggttcaagaggagaaagaggtcttcctggtgttgctggagccgttggagagccaggaccattgggaattgctggacctccaggtgctaggggaccacctggcgctgtgggatctccaggcgtgaatggtgcaccaggagaggcaggaagggatggtaatccgggtaacgatggacctccaggtagagatgggcaaccaggacacaagggggaaaggggttatccaggaaatattggacctgttggagctgcaggtgctccaggtcctcatggaccagttggacctgcaggaaaacatggaaatagaggagagactggtccttcaggtcctgtgggaccagcaggtgctgttggtcctagaggtccatcaggaccacaaggtattagaggagataagggagagccaggagaaaagggaccaagaggtttacctggtttgaaaggacataatggattgcaggggcttccaggtattgctggccatcacggtgatcagggagctccaggttctgtaggtccagcaggtccaaggggaccagctggtccttctggacctgctggtaaggatggtaggactggtcatccaggaactgtgggaccagcaggaattaggggtcctcaagggcatcaaggtcctgctggtccacctggacctccaggtcctccaggacctcctggtgtttctggtggtgggtatgattttggttacgatggagatttttatagggcatga

SEQ ID NO: 5 - protein, 748 aa, synthetic construct
QGAGAAAAAAGGAGQGGYGGLGGQGAGQGGYGGLGGQGAGQGAGAAAAAAAGGAGQGGYGGLGSQGAGRGGQGAGAAAAAAGGAGQGGYGGLGSQGAGRGGLGGQGAGAAAAAAAGGAGQGGYGGLGNQGAGRGGQGAAAAAAGGAGQGGYGGLGSQGAGRGGLGGQGAGAAAAAAGGAGQGGYGGLGGQGAGQGGYGGLGSQGAGRGGLGGQGAGAAAAAAAGGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAAGGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAAGGAGQGGYGGLGGQGVGRGGLGGQGAGAAAAGGAGQGGYGGVGSGASAASAAASRLSSPQASSRVSSAVSNLVASGPTNSAALSSTISNVVSQIGASNPGLSGCDVLIQALLEVVSALIQILGSSSIGQVNYGSAGQATQIVGQSVYQALG

SEQ ID NO: 6 - DNA, 2247 bp, synthetic construct
  source 1..2247: mol_type = other DNA
caaggagctggagctgcagctgcagctgctggaggagctggacaaggtggatatggtggtttgggaggtcaaggtgctggacaaggaggttacggaggtcttggaggacagggagctggacaaggtgctggtgctgcagctgctgctgctgctggaggagctggtcaaggaggttacggtggtttaggttcacaaggagctggtagaggaggacagggtgcaggtgctgctgctgcagcagcaggaggtgctggacagggcggttatggtggtttgggttcccaaggagctggaaggggaggattaggtggacagggagctggtgctgctgctgctgctgctgcaggaggtgctggtcagggtggctatggtggtttggggaatcaaggagctggaaggggtggtcaaggagctgcagcagctgctgctggaggtgctggacaaggcggttacggaggtttgggatctcagggtgcaggaagaggaggtcttggcggacagggagctggagctgctgccgctgctgctggaggtgcaggacaaggcggttatggtggtcttggtggtcaaggagcaggacaaggtgggtacgggggacttggaagtcagggtgctggtaggggaggacttggaggacaaggtgctggggcagctgctgcagctgctgctggaggtgctggacaaggaggtcttggtggacagggagcaggacaaggtgctggagcatctgcagctgcagcaggtggagcagggcagggaggatacggaggacttggttctcaaggtgctggtagaggaggtgaaggagcaggtgctgctgcagctgcagctggaggcgctggacaaggaggctatggaggacttggtggacagggggctggacagggtggttatggaggtcttggatcccagggagctgggaggggaggactcggaggacaaggagctggagctgcagctgctggtggtgctggacaaggtggacttggtggacaaggagctggccaaggtgctggagctgcagctgctgctgctggtggtgcaggtcaaggaggatatggaggacttggaagtcaaggagctggaagaggaggtcttggtggtcaaggtgccggagcagttgctgccgctgctgctggaggtgctggtcagggtggatatggaggtcttggttctcagggtgcaggaagaggaggtcaaggagcaggtgctgcagctgcagctgcaggtggagctggtcaaagaggatacggaggacttggaaaccagggtgctggtagaggaggccttggaggtcagggagctggggctgctgctgctgctgcagctggtggtgctggacaaggtggatatggtggactcggaaatcagggtgctggaagaggtggacaaggtgctgcagctgcagctggcggagcaggacaaggtggatatggtggtttgggttcacagggagcaggaagaggaggacagggagctggtgctgctgctgcagcagctgttggagcaggacaagaaggaattagaggacaaggagctggtcagggaggatatggaggtttgggatctcaaggttcaggtaggggtggacttggtggacaaggagcaggagcagctgctgctgctgctggtggagctggacaaggaggattgggaggacaaggagctggtcaaggagctggagctgctgccgcagctgctggaggagtgagacaaggtgggtatggtggtttgggttcacaaggagcaggtagaggtggacaaggagcaggtgcagcagcagcagctgctggtggtgctggtcaaggaggttacggtggacttggaggtcaaggagttggtaggggaggtcttggtggtcaaggggctggtgctgctgccgctggtggagctggacaaggtggttatggtggtgttggttctggagcttctgctgctagtgctgctgcatcaagactttcttctcctcaagcttcttctagagttagcagcgctgttagtaaccttgtagcttcaggtccaactaattctgctgctctttctagtactatttctaatgttgtgagccaaatcggagcttcaaatccaggattgtctggttgtgatgttttaattcaagctttgttggaggtggtttctgctcttattcagattttgggttcttcttctatcggtcaagttaactatggatctgctggtcaggctacacaaattgttggtcagtctgtttaccaggctcttgggtga

SEQ ID NO: 7 - protein, 636 aa, synthetic construct
ARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 8 - DNA, 1911 bp, synthetic construct
  source 1..1911: mol_type = other DNA
gcaagagcaggatctggtcagcaaggacctggtcaacaaggtccaggacagcagggaccaggacaacaaggtccttatggacctggtgcttcagcagctgctgctgctgctggtggttatggaccaggatctggacaacagggcccttctcagcaaggacctggacagcagggtcctggtggtcaaggaccttacggtcctggagcttctgctgctgctgctgctgcaggtggatatggtcctggtagtggtcaacaaggtccaggaggacaaggaccatacggccctggtagctcagctgctgcagcagctgctggtggtaatggcccaggaagtggacaacaaggtgctggacaacagggtccaggacaacaaggacctggtgcatcagccgctgctgctgctgctggaggttatggaccaggttctggacaacaaggtccaggacaacaaggaccaggaggtcaaggaccttatggaccaggagctagtgctgcagcagctgctgctggaggttatggtcctggaagtggtcaaggacctggacaacaaggacctgggggtcaaggtccttatggacctggagcttccgctgctgctgcagcagccggaggttatggtccaggaagtggtcagcaaggaccaggacagcagggaccaggacagcaaggtcctggaggtcagggaccatatggtccaggagcttctgctgctgctgcagctgctggtggatatggaccaggttatggacaacaaggacctggacaacaaggacctggagggcaaggtccatatggaccaggagcttctgctgctagtgctgcttcaggagggtatggaccaggttctggacaacaaggaccaggacaacaaggtccaggtggtcaaggaccttatggaccgggtgcttctgcagctgccgcagctgctggaggatatggtcctggttcaggtcaacaaggacccggtcaacaaggaccaggtcagcaaggtccaggacaacagggtcctggaggtcagggtccttatgggccaggagcatctgctgcagctgctgctgctggtggttatggaccaggctctggacaacaaggacctggtcaacaaggacctggacagcagggtcctggacagcaaggtccaggacaacaaggaccaggacagcaaggtccaggtcagcaaggacctggacaacaaggaccaggtcaacaaggacctggtggtcagggtgcttatggtccaggtgctagtgctgctgccggagcagctggaggctatggacctggatctggtcagcagggacctggtcaacaaggacctggtcaacagggaccaggtcaacaaggaccagggcaacagggacctggacaacaaggacctggacaacaaggtccaggtcagcagggaccttatggacctggtgcttcagctgctgctgcagctgctggaggatatggcccaggttctggacaacagggacctggacaacaaggtccaggtcaacaaggtccaggtggccaaggaccatacggaccaggagctgcatctgcagctgtttctgttggaggatatggacctcaatcatcatctgttcctgtggcatcagctgttgcttcaagattgtcttcaccagctgcttcatctagagttagcagtgcagtttcttccttggtttcttctggacctactaaacatgctgctctctctaatactattagtagtgttgtttctcaggtttctgcttctaatccaggtttatcaggatgcgatgttcttgttcaagcacttttggaagttgtttccgctttggtttcaattttaggatcatcttctattggtcaaattaattatggcgcttctgcccaatatactcagatggtcggacaatcagttgctcaagctcttgcttaa

SEQ ID NO: 9 - protein, 284 aa, synthetic construct
MDISNLPPHIRQQILGLISKPQQNNDESSSSNNKNNLINNEKVSNVLIDLTSNLKIENFKIFNKESLNQLEKKGYLIIDNFLNDLNKINLIYDESYNQFKENKLIEAGMNKGTDKWKDKSIRGDYIQWIHRDSNSRIQDKDLSSTIRNINYLLDKLDLIKNEFDNVIPNFNSIKTQTQLAVYLNGGRYIKHRDSFYSSESLTISRRITMIYYVNKDWKKGDGGELRLYTNNPNNTNQKELKQTEEFIDIEPIADRLLIFLSPFLEHEVLQCNFEPRIAITTWIY

SEQ ID NO: 10 - protein, 490 aa, synthetic construct
APDEEDHVLVLHKGNFDEALAAHKYLLVEFYAPWCGHCKALAPEYAKAAGKLKAEGSEIRLAKVDATEESDLAQQYGVRGYPTIKFFKNGDTASPKEYTAGREADDIVNWLKKRTGPAASTLSDGAAAEALVESSEVAVIGFFKDMESDSAKQFFLAAEVIDDIPFGITSNSDVFSKYQLDKDGVVLFKKFDEGRNNFEGEVTKEKLLDFIKHNQLPLVIEFTEQTAPKIFGGEIKTHILLFLPKSVSDYEGKLSNFKKAAESFKGKILFIFIDSDHTDNQRILEFFGLKKEECPAVRLITLEEEMTKYKPESDELTAEKITEFCHRFLEGKIKPHLMSQELPDDWDKQPVKVLVGKNFEEVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDLEEDDDQKAVKDEL

SEQ ID NO: 11 - DNA, 855 bp, synthetic construct
  source 1..855: mol_type = other DNA
atggatatttctaatttgccaccacatatcaggcagcaaattcttggcttgatttctaagccacaacaaaataacgatgagagttcttcatcaaacaataaaaataaccttattaacaacgagaaggtgtcaaatgttttgattgatcttacttccaatcttaagattgagaacttcaaaatctttaacaaggagtctctcaatcagcttgaaaagaagggataccttattattgataatttcttgaatgatttgaataaaattaatttaatttatgatgaatcctataatcaatttaaagaaaataaacttatagaagctggaatgaacaagggaactgataagtggaaagataagtcaatcagaggagattacatacagtggattcatcgggattctaatagtagaattcaggataaggatctttcatctacgattagaaatattaattatcttctcgataaacttgacttgattaaaaatgagtttgataatgtcattccaaattttaattcaatcaaaacacaaactcaattagcagtgtacttgaatggcggtaggtacattaagcatagagattctttctattcttctgaaagtcttactatttctaggaggattactatgatatactatgtgaataaggattggaagaagggagatggaggtgagttgagattgtatacaaacaatccaaataatacaaatcaaaaagagttgaagcagactgaagaattcattgatattgaacctattgctgatcgtttacttatttttctttcccccttccttgaacatgaggtgcttcaatgcaatttcgagccaaggatagctattactacttggatatactaa

SEQ ID NO: 12 - DNA, 1473 bp, synthetic construct
  source 1..1473: mol_type = other DNA
gctcctgatgaagaggaccatgtacttgttttgcataagggtaattttgatgaagctcttgcagcacataaatatctattggtggagttctatgcaccttggtgtggacactgcaaggctttggctccagagtacgctaaggctgcaggaaaacttaaggctgagggttctgagattagactcgctaaggttgatgctactgaggaatcagatttggctcagcaatatggagttaggggatacccaactattaaatttttcaaaaatggggatactgcatcaccaaaagaatacactgccgggagagaagctgatgatattgttaattggttgaagaagaggactggccctgctgcttctactttgtctgatggagcagctgcagaggcacttgttgaatcttcagaagttgctgttattggatttttcaaagatatggagtccgattctgctaagcaattctttctggcagcagaagtcattgatgatattccatttggaatcacttcaaattctgatgttttttctaagtatcagcttgataaggatggggttgttctttttaaaaaattcgacgagggaaggaataacttcgagggagaggtgacaaaagagaagcttcttgatttcattaagcataatcagctccctcttgttattgaatttaccgaacagactgcacctaagatcttcggtggagaaattaaaactcatattttgctttttttgccaaaatctgtttcagattatgaaggaaaactttctaattttaagaaagctgctgaatcatttaagggtaagattttgtttatttttattgactcagatcatactgataatcaaaggatcttggaattttttgggttaaagaaggaggaatgtcctgctgttagacttattactttggaggaagaaatgacaaagtacaagcctgaaagtgatgaattgacagcagaaaagattactgaattctgtcatcgtttcctggaaggtaagattaagccacatttgatgtcccaagaacttcctgatgattgggataagcaacctgttaaggttctcgttggtaagaattttgaagaggtggcttttgatgagaaaaagaatgtctttgttgagttctatgcaccttggtgtggacactgtaaacaattggctccaatttgggacaagctcggtgaaacctataaggatcacgaaaacattgttattgctaagatggattctacagctaatgaagtggaggctgttaaggtgcattcttttccaacacttaaatttttcccagcttcagctgataggactgtaatagattataacggagagcgtactctagatggtttcaaaaagtttctggagtcaggtggtcaagatggagctggagatgatgatgatcttgaagatctcgaggaagctgaggaaccagatcttgaggaggatgatgatcaaaaagctgttaaggatgagttgtga

SEQ ID NO: 13 - protein, 722 aa, synthetic construct
SDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYTYPEHERELGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 14 - DNA, 2145 bp, synthetic construct
  source 1..2145: mol_type = other DNA
tctgataggccacgtggaagagatcctgtgaacccagagaagcttttggttattactgttgctactgcagagactgaaggataccttagatttcttcgttcagcagagttctttaattatactgttagaactttgggattgggtgaagaatggagaggaggagatgttgctaggactgttggtggtggacaaaaagttaggtggttgaagaaggaaatggaaaagtatgctgatagggaggatatgattattatgtttgtggattcctatgacgttattcttgctggatctcctaccgagcttcttaaaaagtttgttcaatcaggatcaagacttctctttagtgcagagagtttctgttggccagaatggggtctcgctgaacaatacccagaagttggaactggaaagagattcttgaattctggaggatttattggatttgctacaactattcatcaaattgttagacagtggaagtataaggatgatgatgatgatcagcttttttatactaggctttatttagatcctggtctcagagaaaaactttctttgaatcttgatcataagagcagaatcttccaaaatcttaacggagctttggatgaggttgttcttaaatttgatagaaacagagttaggattagaaatgttgcttatgatactttgcctattgttgttcatggaaatggaccaactaagcttcaacttaactatcttggtaactacgttcctaatggatggactccagaaggtggttgtggattttgcaaccaggatagaaggacacttcctgggggacaaccccctccaagggtgttcttggctgtttttgttgaacaaccaactccatttttgcctcgttttttgcaaagactgctacttcttgattatccacctgatagagttactttgttcttgcataataatgaagtttttcatgaaccacatattgcagattcttggccacaactccaagatcactttagcgctgttaaacttgttggtccagaagaagctttgtctcctggtgaagcaagagacatggccatggatctctgcagacaagacccagaatgtgaattttattttagtttggatgctgatgctgttttgactaatttgcagacattgagaattcttatcgaagaaaacaggaaagttattgctcccatgctttcaaggcatggaaagctctggtctaatttctggggtgcactttctccagacgaatactatgcaagatctgaagattatgttgagttggttcagagaaagagagttggagtttggaacgttccctatatttcacaggcttacgttattagaggagataccttgcgtatggaattgccacagagggatgttttctctggctctgatacagatccagatatggctttctgcaaatcttttagagacaaaggtatttttttgcatttgtcaaaccaacatgaattcggtagacttcttgctacctctaggtatgatactgaacaccttcatccagatctctggcaaatttttgacaatcctgttgactggaaggagcaatatattcacgagaactactctagagctcttgaaggagagggtattgttgagcaaccttgccctgatgtgtactggtttcctttgctctctgagcaaatgtgtgatgaacttgttgctgaaatggaacattacggccaatggtcaggtggtaggcatgaggattcaagacttgctggtggttacgaaaacgttccaacagtggatatccatatgaagcaagttggttatgaagatcagtggcttcaattgcttaggacttatgttggacctatgactgaatcactttttccaggatatcacacaaaggctagagctgtgatgaattttgttgttagatatagaccagatgaacagccatcattgagaccacatcatgattcttctacttttactttaaatgtggctcttaaccataagggactggattacgaaggaggaggttgcagatttcttagatatgattgtgttatttcctctccaagaaaagggtgggcattgttacatccaggtagacttacacattaccatgaaggacttcctactacttggggaactcgttatattatggtttctttcgttgatccttaa

SEQ ID NO: 15 - protein, 2943 aa, synthetic construct
MATTLISKLTLSSAFLGQQFSSRGNSMRSAPAGLFLRGPRQGAGAAAAAAGGAGQGGYGGLGGQGAGQGGYGGLGGQGAGQGAGAAAAAAAGGAGQGGYGGLGSQGAGRGGQGAGAAAAAAGGAGQGGYGGLGSQGAGRGGLGGQGAGAAAAAAAGGAGQGGYGGLGNQGAGRGGQGAAAAAAGGAGQGGYGGLGSQGAGRGGLGGQGAGAAAAAAGGAGQGGYGGLGGQGAGQGGYGGLGSQGAGRGGLGGQGAGAAAAAAAGGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAAGGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAAGGAGQGGYGGLGGQGVGRGGLGGQGAGAAAAGGAGQGGYGGVGSGASAASAAASRLSSPQASSRVSSAVSNLVASGPTNSAALSSTISNVVSQIGASNPGLSGCDVLIQALLEVVSALIQILGSSSIGQVNYGSAGQATQIVGQSVYQALGGSGEGRGSLLTCEDVEENPGPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRTYPEHEREPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRAGSGEGRGSLLTCEDVEENPGPQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGTYPEHERESKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRA

SEQ ID NO: 16 - DNA, 8784 bp, synthetic construct
  source 1..8784: mol_type = other DNA
atggctaccactcttatatctaagttgactctttcatcagctttccttggacaacagttttcatctagaggtaattcaatgagatcagcaccagctggactttttcttaggggaccaagacaaggagcaggtgctgctgcagcagcagctggaggtgctggacaaggtggttatggaggacttggaggacaaggagcaggacagggtggatatggtggattgggaggacaaggtgctggacaaggagctggagctgctgctgctgcagcagctggaggtgctggtcagggtggatatggaggattgggaagccaaggagctggtaggggtggtcaaggagctggtgctgctgctgcagcagctggtggagcaggtcagggcggttatggaggcttgggttctcaaggagctggaaggggtggcttgggtggccaaggtgccggtgctgctgctgctgctgctgctggtggtgctggtcaaggcggatatggaggacttggaaaccaaggtgctggccgtggtggacagggagctgctgctgctgctgcaggaggagctggtcagggtgggtatggtggtttgggttcacagggagctggaaggggtggacttggaggacagggtgcaggagcagctgctgctgcagctggtggtgcaggtcaaggtggatacggtggtcttggtggacaaggagctggtcagggtggctacggtggacttggaagtcaaggagctggaagaggtggtcttggaggtcaaggagccggtgctgctgctgcagctgcagctggtggagctggacaaggcggtctgggtggccaaggtgctggacagggagcaggtgcatctgcagctgcagctggtggagctggtcaaggtggctatggtggattgggttctcagggagctggtagaggtggagaaggagctggagctgctgcagctgctgctggaggagcaggtcagggtggttacggaggtttaggaggtcaaggagccggacaaggaggatatggaggtcttggttctcaaggggcagggagaggaggtttaggtggacagggagctggtgctgcagctgctggaggagctggtcagggaggacttggaggacaaggtgcaggtcaaggtgctggtgcagctgctgctgccgctggaggtgctggacagggagggtatggaggccttggtagccagggtgcaggcaggggaggtttgggaggacagggtgctggtgctgtggcagcagctgccgcaggaggtgctggacaaggaggatatggaggacttggatctcaaggtgctggtagaggtggtcaaggagctggagctgctgctgctgcagctggaggagccggtcaaagaggatacggtggactaggtaatcaaggagctggaaggggaggattgggtggtcagggagctggagcagcagctgcagcagctgctggaggagcaggtcaggggggttatggaggattggggaatcaaggtgcaggaagaggtggacaaggggctgctgcagctgctggtggagctggccaaggaggttacggtggacttggttctcagggagcaggaagaggagggcagggagctggagctgcagctgctgctgctgttggtgctggtcaggaaggtattagaggacagggagctggtcaaggaggttacggaggtttagggtcccagggttctggaagaggaggactgggaggacaaggagcaggtgctgctgctgctgcagctggtggtgctggacaaggaggtcttggaggacaaggagctggacagggagctggtgcagctgctgctgctgctggaggagttagacagggaggatatggaggtttgggatcacaaggtgcaggaagaggtggacagggagctggagctgcagctgctgcggctggtggggctggacaaggtggatatggagggcttggaggccaaggagttggaaggggtgggcttggtggacaaggtgcaggtgctgctgctgctggaggtgctggtcaaggcggttacggaggtgttggttctggagcttcagctgcaagtgctgcagctagtaggctttctagtccacaagcatcatctagagtttcttctgctgtttctaatttggtggcatctggtccaacaaactcggcagcactttcttctactatttctaatgttgtttctcagataggtgcatctaacccaggtctttcaggatgtgatgttttgatacaggctttgcttgaagtggttagtgctcttatacaaattctcggatcctcatcaattggtcaagtgaactacggttctgctggacaagctacacagattgttggtcaatcagtttatcaagcacttgggggttctggtgaaggaaggggtagtcttcttacttgtgaagatgtggaagaaaatcctggaccacagctttcttatggatacgatgaaaagtctactggaggtatatctgttcctggtcctatgggacctagtggtcctagaggtttgccaggacctcctggtgctccaggaccacaaggatttcagggaccaccaggggaaccaggtgaacctggagcttctggaccaatgggtcctagaggtccacctggacctcctggtaaaaatggagatgatggtgaggctggaaagccaggaaggccaggagaaagaggtccaccaggaccacagggtgctcgtggtcttccaggaacagccggtttacctggcatgaagggacatagaggattttcaggtttggatggagctaaaggagatgctggaccagctggacctaaaggagagccaggatctcctggagagaatggtgcacctggccagatgggtccaaggggtcttccaggtgagagaggtagacctggagccccaggtccagcaggcgctagagggaatgacggagccacaggtgcagctggtccacctggacctactggtcctgctgggcctcctggctttcctggagctgtaggtgctaagggtgaggctggacctcaaggtcctcgaggatcagaaggtccacaaggagttaggggagagcctggcccaccaggtccagctggagctgcaggtcctgctggtaatccaggagctgatggacaacctggagctaaaggtgctaacggagctcctggaattgctggagcaccaggttttcctggtgctagaggaccatcaggaccacaaggaccaggtggtcctccaggacctaaaggtaatagtggagaaccaggtgctcctggttctaaaggagatactggtgctaagggagaaccaggccctgttggagtccaaggtccacctggaccagctggagaagaaggaaagaggggagctagaggcgaaccaggacctactggattgccaggtcctccaggtgaaagaggaggtccaggttctaggggtttcccaggagcagatggagtagctggacctaagggacccgctggtgaaagaggttcacctggacctgcaggtcctaagggttcaccaggtgaagcaggtagacctggtgaagcaggtttgcctggagctaagggtttgacaggaagtccagggtcacctggaccagatggaaagacaggacctcctggtccagctggtcaagatggaagacctggtcctccaggaccaccaggtgcaagaggacaagctggagttatgggttttcctggtccaaagggagctgctggagagccaggtaaagctggtgaaagaggtgttccaggtcctcctggtgctgttggaccagctggtaaagatggagaagctggagctcaaggtccacctggtcctgcaggaccagctggagaaagaggcgaacaaggtcctgctggttcgccaggatttcagggtttaccaggtcccgctggtcctccaggtgaagctggaaaacctggagaacaaggtgtgcctggagatttgggagctccaggaccttctggtgcaagaggtgagcgtggtttccctggagaaaggggtgttcaaggtccacctggacctgctggtcctagaggagctaacggagctccaggaaatgatggtgcaaagggtgatgctggtgcacctggtgctcctggatctcaaggtgctccaggtcttcagggtatgccaggagagaggggagctgctggattacctgggcctaaaggtgatagaggagatgctggtccaaagggtgctgatggtagtccaggtaaagatggtgttagaggacttacaggccctattggtccacctgggccagctggtgcaccaggtgataagggagaaagtggaccaagtggaccagcaggaccaaccggtgctagaggagcaccaggtgatagaggagaaccaggtccaccaggaccagctggttttgctggtcctccaggagctgatggacaaccaggagctaaaggtgagcctggagatgctggagctaaaggagatgctggtccaccgggaccagcaggtccagcaggcccaccaggtccaattggaaacgttggtgcacctggcgctaagggtgccagaggaagcgcaggtccaccaggagcaactggctttccaggtgctgcaggtagagttggaccaccaggaccttctggaaacgctggacctcctgggcctccaggacctgctggaaaggaaggagggaagggtcctaggggagagactggaccagctggtagaccaggtgaggttggaccaccaggtcctccaggcccagctggtgaaaagggtagtcccggtgctgatggaccagcaggagctccaggaacaccaggacctcaaggtattgctggtcaaagaggtgttgttggtttgcctggtcagagaggagaaagaggatttccaggattgccaggaccttctggtgagcctggtaaacagggtccatcaggtgcttctggggaaagaggaccacctggtcctatgggaccaccaggtttggctggtcctcctggtgaatcaggtagggaaggagctcccggagctgaaggatcaccaggaagagatggatctcctggagctaaaggagatagaggagaaacaggtccagctggaccaccaggagcacctggtgctcctggtgctcctggacctgttggtccagctggtaaatcaggagatagaggtgaaactggacctgctggaccagctggtccagttggacctgttggtgctagagggccagcaggaccacagggtcctagaggagataagggagagactggtgaacaaggagatagaggaatcaaaggtcatagaggatttagtggacttcaaggaccacctggccctcctggttctcctggagaacaaggcccatctggtgcttctggacctgctggcccaaggggaccacctggatctgctggtgcccctggtaaagatggacttaatggattgccaggtccaattggtcctcctggtccaagaggaaggacaggagatgctggacctgttggtcctcctgggccaccaggaccacctggacctcctggacctccaagtgctggttttgatttctcttttttaccacagccaccacaggaaaaagctcatgatggtggaagatactatagagctggttcaggtgagggtagaggatccttacttacatgtgaagatgttgaggaaaatcctggaccacagtacgatggaaaaggtgttggacttggtccaggtccaatgggattgatgggaccaagaggacctccaggagctgcaggagctccaggaccacagggatttcaaggtcctgcaggagagcctggagagccaggtcaaactggacctgcaggagctagaggtccagctggacctccaggtaaagctggagaagatggtcatccaggaaagccagggaggccaggtgaaaggggtgttgttggtccacagggggctagaggcttccctggtacacctggtcttccaggatttaaaggtattagaggtcataatggtttagatggattgaagggtcaaccaggagctccaggtgttaagggggaaccaggagcaccaggtgaaaatggaactcctggtcagactggagctagaggacttccaggagaaagaggtagagtgggtgcacctggtccagcaggggctcgtggtagtgatggttccgttggacccgtcggacctgcaggtccaattggatcagcaggaccacctggattcccaggagctccaggtccaaaaggcgagattggtgctgttggaaatgctgggcctgctggacctgctggtcctagaggagaggttggacttccaggtttgtccggaccagtgggaccacctggaaatccaggagctaatggtcttactggagctaaaggagctgcagggttgcctggtgttgctggagctccaggacttcctggacctagaggaattcctggtccagttggagctgctggtgctactggtgctagaggacttgttggagaaccaggtccagcaggatctaagggagagtcaggtaataaaggtgagccaggaagtgctggtccacaaggtccaccaggaccttctggtgaggagggtaagaggggtccaaatggtgaagctggatcagctggacctccaggaccacctggacttaggggtagccctggttcaagaggactgcctggggctgatggaagagctggagttatgggacctcccggtagtaggggagcatccggaccagctggagtaaggggacctaatggtgatgctggaagaccaggagaacctggattaatgggtcctaggggtctcccaggatctccaggtaacattggtcctgctggtaaagaaggaccagttggtcttccaggcattgatggtagaccaggaccaattgggccagctggtgctcgtggcgaacctggtaatataggattcccaggtcctaagggaccaaccggtgatccaggtaaaaatggtgataaaggtcatgctggattggccggagctaggggagctccaggtccagatggaaataatggtgctcagggaccaccaggaccacagggtgttcaaggtggaaaaggagaacagggtcctcctggtcctccaggtttccaaggacttcctggaccttctggtccagcaggtgaggttggtaaaccaggagagagaggattgcacggagaatttggtttgccaggaccggctggtcctaggggtgaaagaggaccacctggtgaatctggagctgctggaccaactggtcctattggttcaaggggaccttctggacctccaggtccagatggaaataaaggagagccaggagtggttggagctgttggaacagctggaccaagtggaccttcaggactcccaggagagaggggcgctgctggtattcctggtggaaaaggtgagaagggtgagcctggacttagaggagaaataggaaatccaggcagggatggtgcacggggagctcctggagctgttggtgccccaggaccagccggagcaacaggagataggggagaggctggtgctgctggtccagctggacctgcaggacctaggggttcaccaggagaaagaggtgaggttggtccagctggtcctaatggatttgctggtcctgctggtgctgctggtcaacctggagctaagggtgagaggggtgcaaaaggacctaaaggtgaaaatggtgttgttggtcctactggaccagttggagctgctggacctgctggaccaaatggtccacctggtccagctggttctagaggagatggtgggccacctggaatgactggattcccaggtgctgctggaaggactggaccaccaggccctagtggaatttctggaccacctggtcctcctggaccagcaggtaaggaaggtttgaggggaccaagaggggatcagggacctgtaggtagaactggtgaggttggtgctgttggcccaccaggtttcgctggcgaaaagggaccttcaggtgaagctggtacagctggtcctcctggtactcctggtccacaaggtttgcttggtgctcctggtattcttggtcttccaggttcaagaggtgagagaggtcttcctggagtggctggagctgttggagaaccaggtccattgggtatagctggacctccaggcgctagaggcccacctggtgcagtcggatcaccaggtgttaacggagctccaggtgaggcaggtagagatggaaatcctggaaatgatgggcctcctggtagggatggacagccaggtcataaaggtgaaagaggataccctggaaatatcggtcctgttggtgctgctggtgcaccaggaccacatggtcctgttggtcctgctggaaagcatggtaatcgaggagaaactggaccatctggaccagttggtccagcaggtgctgttggaccacgaggaccttcaggaccacagggaattaggggtgataagggcgagcctggtgaaaagggacctaggggtcttccaggtttgaaaggtcataacggactgcaaggacttccaggaattgctggtcaccacggtgatcaaggagccccaggttctgttggtccagctggaccaagaggaccagcaggtccatcaggtccagctggaaaagatggtagaactggacatccaggcacagttggtccagctggtattaggggacctcaaggtcatcaaggaccagctggacctccaggaccacctggtccaccaggaccaccaggtgtttctggaggaggctacgattttggttatgatggtgatttttatagggcttaa

SEQ ID NO: 17 - protein, 1578 aa, synthetic construct
MATTLISKLTLSSAFLGQQFSSRGNSMRSAPAGLFLRGPRMDISNLPPHIRQQILGLISKPQQNNDESSSSNNKNNLINNEKVSNVLIDLTSNLKIENFKIFNKESLNQLEKKGYLIIDNFLNDLNKINLIYDESYNQFKENKLIEAGMNKGTDKWKDKSIRGDYIQWIHRDSNSRIQDKDLSSTIRNINYLLDKLDLIKNEFDNVIPNFNSIKTQTQLAVYLNGGRYIKHRDSFYSSESLTISRRITMIYYVNKDWKKGDGGELRLYTNNPNNTNQKELKQTEEFIDIEPIADRLLIFLSPFLEHEVLQCNFEPRIAITTWIYGSGEGRGSLLTCEDVEENPGPAPDEEDHVLVLHKGNFDEALAAHKYLLVEFYAPWCGHCKALAPEYAKAAGKLKAEGSEIRLAKVDATEESDLAQQYGVRGYPTIKFFKNGDTASPKEYTAGREADDIVNWLKKRTGPAASTLSDGAAAEALVESSEVAVIGFFKDMESDSAKQFFLAAEVIDDIPFGITSNSDVFSKYQLDKDGVVLFKKFDEGRNNFEGEVTKEKLLDFIKHNQLPLVIEFTEQTAPKIFGGEIKTHILLFLPKSVSDYEGKLSNFKKAAESFKGKILFIFIDSDHTDNQRILEFFGLKKEECPAVRLITLEEEMTKYKPESDELTAEKITEFCHRFLEGKIKPHLMSQELPDDWDKQPVKVLVGKNFEEVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDLEEDDDQKAVKDELGSGEGRGSLLTCEDVEENPGPSDRPRGRDPVNPEKLLVITVATAETEGYLRFLRSAEFFNYTVRTTYPEHERELGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 18 - DNA, 4713 bp, synthetic construct
  source 1..4713: mol_type = other DNA
atggcaacaacacttattagtaaactcactctttctagtgcttttcttggacagcaattttctagcaggggaaattctatgagaagtgctccagccggtttatttttgcgcggtcctagaatggatataagtaacttgccaccacatattagacagcaaattcttggtcttatctcaaagcctcaacagaacaatgatgaatcttcatcatctaataataagaataatcttatcaataacgaaaaggtttctaatgttcttattgatcttacttctaatttgaagattgaaaattttaaaatttttaataaagagtcacttaatcaactcgaaaaaaagggatacctcataattgataatttcttaaatgaccttaataagattaatcttatttatgatgaatcttataaccaatttaaggaaaacaagcttattgaagctggtatgaataagggtacagataaatggaaagataagagtattagaggggattatattcagtggattcatagagattccaattctagaattcaagataaggatctttcaagtacaattagaaatattaattatttgttggacaagttggatcttattaagaatgagtttgataacgttatccctaattttaattctatcaagactcaaacccaattggctgtatatttgaacggaggaagatacattaaacatagggatagtttttattcctcagaatctttgactattagcagaagaattactatgatttattatgtcaataaagactggaaaaagggagatggaggagagcttagactgtacactaataacccaaacaatactaatcaaaaagagttgaaacaaactgaagaatttattgatatagaaccaatagcagacagattgcttatttttttgtctccatttcttgaacatgaggttcttcaatgtaattttgaaccacgtattgctattactacatggatttatggatctggcgagggtaggggttcactccttacttgtgaggatgttgaagagaatcctggaccagcaccagatgaggaagatcatgttttggttcttcataaaggaaattttgatgaagctttggctgctcacaaatatttgcttgttgaattctatgctccttggtgtggtcattgcaaggcattggcccctgagtatgctaaggcagctggaaagttgaaggccgagggatctgaaattagacttgcaaaggttgacgctactgaggaatctgatttggcacaacaatatggtgttagaggttacccaactattaagttctttaagaatggtgacactgctagtcctaaggaatataccgctggtagagaggccgatgatatcgtaaattggcttaagaaaaggacaggaccagcagcttcaacattgtcagatggtgctgctgctgaagctttagtcgaatcttcagaagttgctgtgattggattttttaaagacatggaatctgatagtgccaaacagttttttttggctgcagaggtgattgatgatattccatttggaattacttcaaattcagatgtgttttcaaaatatcagcttgataaggacggagttgttttgttcaaaaagttcgatgaaggaagaaataattttgaaggtgaagtgactaaagaaaagcttcttgattttattaagcacaatcaattgccattggttattgaatttactgagcaaactgctccaaagatttttggtggtgaaattaagactcatattcttttgttcttgcctaagtctgttagtgattatgaaggtaagttgagcaactttaaaaaggctgctgaatcttttaagggaaaaattctttttatcttcattgatagcgatcacacagataatcagagaatattggaattcttcggtttgaagaaggaagaatgccctgctgttaggttgattacactggaggaggagatgactaagtacaagcctgaatctgatgagcttactgctgaaaagatcactgaattctgccacagatttcttgaggggaagattaagccacaccttatgtctcaggagttacctgatgattgggataaacaacctgttaaggttctcgtgggtaagaactttgaagaggttgctttcgatgaaaaaaaaaatgttttcgttgaattctatgcaccttggtgcggtcattgtaaacagctagcaccaatttgggataaacttggggaaacttataaggatcatgagaatattgttatagctaaaatggattcaacggctaatgaagttgaagctgttaaagtccattcatttcctactcttaagttctttcctgcttcagcagaccgtaccgttattgattacaatggtgagagaacattggatggctttaaaaaatttttggaatctggcggacaggatggagctggagatgatgatgacttggaggatttagaggaggccgaagagcctgatttggaagaagatgatgatcaaaaagcagtgaaggatgaactaggttcaggagagggtagggggagtttgttgacttgcgaggatgtagaagaaaaccctggtccatctgatagacctagaggtagagatcctgttaacccagagaagcttttggttattacagtggctacagctgagacagaaggatatcttaggtttctaaggtctgctgaattttttaattatacagttagaacattgggacttggggaggaatggagaggtggggatgttgctcgaactgtgggaggaggtcaaaaggttcgttggttgaagaaagaaatggaaaaatatgcagatagagaagatatgattattatgtttgttgatagttacgatgttattttggctggaagccctacagaattgttaaagaagtttgttcaatctggctcaaggcttttgttctccgcagagagtttctgctggccagagtggggactagctgaacagtatcctgaggtgggtactggtaagaggtttctcaattccggtggttttattggcttcgcaactactattcaccaaattgttagacaatggaagtataaagatgatgatgatgatcaacttttttatacaagactttaccttgacccaggtttgagagaaaagttgtctctgaacttggatcacaagtctagaattttccaaaatctcaatggagctttggatgaagttgttttgaaatttgatagaaatagggttaggattcgtaatgttgcctatgacacacttcctattgtagtgcatggaaatggacctactaagcttcagttgaactatttaggtaactatgtgcctaacggatggactccagaaggtggttgtggattttgtaatcaagatcgaagaactttgccaggaggacaacctccaccaagggtttttcttgctgttttcgttgagcaacctaccccattccttccaagattcttacaaagacttttgttgcttgattatccaccagatagagttactttgttccttcacaataatgaggtgtttcatgagcctcatattgctgatagttggccacaactccaagatcatttctccgcagttaagctcgttggtccagaggaagctttgtctcctggtgaagctagggatatggcaatggatctttgcagacaagatcctgaatgcgaattttacttttctttggatgctgatgctgtgcttacaaatcttcagactctaagaattttgatagaggagaacaggaaagttattgctccaatgcttagtaggcatggtaaattgtggagtaatttctggggagctctttctccggatgaatattatgctagatcggaagattacgtggagcttgttcaacgtaagagagttggtgtatggaatgttccatatatctcacaagcttacgttatcagaggagatacattgagaatggaacttcctcagagagatgtttttagcggatcagataccgatcctgatatggcattttgtaaatcattcagagataagggaattttccttcatctatctaatcagcacgaattcggaaggttgcttgctacatcaagatatgatactgagcacctgcatccagatttgtggcaaattttcgataatccagtggattggaaagaacaatacatacatgaaaattattctagagctcttgaaggtgagggaattgtcgaacaaccttgcccagacgtctattggtttccacttctttctgagcaaatgtgcgatgaactagtggcagagatggaacattacggacaatggtctggaggacggcatgaggattcaagattggctggagggtacgagaatgtgccaactgtcgatattcatatgaagcaagttggatatgaagatcagtggttgcaacttttaagaacatacgttggtcctatgactgaatcattgtttccaggataccacacaaaagcaagagcagttatgaatttcgttgttagatacagaccagatgagcaaccttctttaagaccacatcatgattcttctacatttactctcaatgttgctttgaatcacaaaggtcttgattatgagggaggaggatgcaggtttctgagatatgattgtgtaatttcatcgcctcgtaaaggatgggctttgctccatccaggaagacttactcactatcatgaaggactccctactacatggggtactagatatattatggtttcatttgttgatccttga

SEQ ID NO: 19 - protein, 2831 aa, synthetic construct
MATTLISKLTLSSAFLGQQFSSRGNSMRSAPAGLFLRGPRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGTYPEHEREPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALAGSGEGRGSLLTCEDVEENPGPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARTYPEHEREGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRAGSGEGRGSLLTCEDVEENPGPQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRA

SEQ ID NO: 20 - DNA, 8448 bp, synthetic construct
  source 1..8448: mol_type = other DNA
atggctactactttgatttcaaagttaaccctttctagtgctttcctcggccaacagttttcttctaggggtaattctatgagatctgcacctgcaggattgtttcttcgtggaccaagagctagagcaggatcgggtcaacaaggaccaggacaacagggaccaggacaacagggtccaggtcaacaaggaccatatggtcctggagcatcagcagctgctgctgcagctggtggatacggtccaggaagcggtcaacaaggtccatcccaacaaggtcctggtcaacaaggaccaggagggcaaggtccttacggacctggtgctagtgcagctgctgcagctgctggaggttacggaccaggttctggtcaacaaggaccaggaggacaaggtccatacggaccaggatcttctgctgcagctgctgctgcaggaggaaatggtcctggatctggacaacaaggagcaggtcaacaaggtcctggccaacaaggtccaggtgcttctgctgctgctgctgcagcaggtggttatggtcccggatcaggacaacaaggtcctggacaacaaggtcctggaggacaaggaccttatggtcctggtgctagtgctgctgctgctgctgctggaggatatggtccaggaagcggacaaggaccaggacagcaagggcctggaggtcagggtccatatggtcctggagcttctgcagctgctgctgctgctggtggatatggaccaggttctggacaacagggtcctggtcaacaaggaccaggacagcagggaccaggaggtcaaggtccatatggacctggagcatcagcagctgcagcagctgcaggtggctatggtcctggatatggtcaacagggacctggacagcagggtcctggaggtcaaggtccttatggtcctggagcttcagctgcttctgcagcttccggtggatatggacctggatctggtcagcaaggccctggtcaacaaggtccaggaggtcaaggaccttatgggcctggagcttctgctgctgcagctgcagctggaggatacggacctggatctggtcagcaaggaccaggtcaacagggtccaggtcaacaaggaccaggtcaacaaggtccaggagggcagggaccatatggacctggagcttcagcagcagctgctgctgctggtggatacggtccaggttcaggacaacagggccctggacaacaaggacctggccaacaaggacctggtcaacaaggtcctggtcaacaaggacctggtcaacaaggaccaggacaacaaggaccaggtcaacagggaccaggtcaacaaggtcctggaggtcagggtgcttatggtccaggtgcttccgctgctgctggtgctgcaggtggttacggacctggatctggacagcaaggaccaggtcaacaaggacctggacaacaaggtccaggacaacaaggacctggacaacaaggtccaggtcaacaaggtcctggtcagcagggtccaggacaacaaggtccttatggaccaggggctagcgctgctgcagcagcagcaggtggatatggaccaggtagtggtcaacaaggtcctggacagcaaggtcctggtcaacaaggtcctggaggtcaaggaccctacggtccaggtgctgcttcagcagctgtgtctgttggtggatatggaccacagtcttcttcagtcccagttgcatctgcagttgcatctagactttcatctccagctgcttcatctagagtttcttctgctgtttcttctcttgtgtcatctggtccaactaaacatgctgcactttctaacacaattagttcagttgtttctcaagtttctgcatctaacccaggactttctggttgcgatgttcttgtgcaagctcttctggaagttgttagtgctttggtttccattttgggttctagctctattggacagatcaattatggtgcttcagcacaatacactcaaatggttggacaaagcgttgctcaggctcttgctggaagcggagaaggaagaggtagtctgcttacatgtgaagatgttgaagaaaatcctggtccacaactttcatatggttatgatgagaaatcaacaggtggtatttctgtgccaggacctatgggtccttcaggccctagaggattgccaggtccacctggtgctcctggtcctcaaggattccaaggaccaccaggtgagccaggtgaacctggagctagtggaccaatgggtcctagaggtccacctggtcctcctggtaaaaatggtgatgatggagaggcaggaaagcctggaagacctggagaaagaggaccacctggacctcaaggagctcggggacttccaggtacagctggattgccaggtatgaagggacacagaggattcagtggcttggatggagctaagggagatgctggtccagctggacctaaaggagagccaggttctccaggagaaaacggagctccaggacaaatgggacctagaggtcttcctggtgaaaggggtaggccaggagcccctggacctgctggtgctagaggtaacgatggagctactggtgctgctggaccaccaggacctactggtcctgcaggtccaccaggttttccaggtgcagttggagcaaagggtgaggctggtccacaaggacctagaggttcagaaggaccacaaggtgttagaggtgaaccaggtccaccgggaccagcaggagccgctggccccgctggtaatcctggtgctgatggtcaaccaggtgctaagggagctaacggtgctccagggattgctggtgctccaggattccctggagctagaggaccttcaggtccacaaggtcctggtggaccacctggaccaaaaggaaatagcggagagccaggtgcacctggctcaaagggagatactggagcaaagggagagcctggacctgttggtgttcaaggtcctcctggacctgctggtgaggagggaaagagaggtgcaagaggtgagcctggtcctacaggactccctggtcctcctggtgaaaggggaggacctggatctaggggttttccaggtgctgatggagttgctggacctaaaggaccagctggagaaaggggatctccaggtccagctgggccaaagggttctcctggagaggcaggaagaccaggtgaagctggattgccaggtgccaagggacttacaggatctcctgggtcaccaggaccagatggaaagactggtcctcctggaccagctggacaagatggaagacctggaccacctggaccacctggagcaaggggtcaagctggtgttatgggttttccaggtccaaaaggtgcagcaggcgagccaggaaaggctggtgaaaggggtgttccaggtccacctggagcagttggtccagctggaaaggatggagaggctggcgctcaaggtcctcctggtcctgctgggccagcaggtgaaagaggagaacaaggacctgctgggtctcctggttttcaaggacttcctggaccagctggtcctccaggtgaagcaggcaagccaggagagcaaggtgttcctggagatcttggtgccccaggtccttctggtgcaagaggagagcgtggattccctggagaaagaggtgtgcaaggtcctccaggtccagctggtccacgtggagctaacggagctcctggtaacgatggagctaaaggagatgctggtgccccaggcgcacctggttcacaaggtgctcctggattgcaaggtatgcctggcgaaagaggtgctgctggacttcctggacctaagggtgacagaggtgatgctggaccaaaaggagctgatggatcacctggtaaagatggagtgagaggtttaaccggtccaattggaccaccaggtcccgctggagctccaggagataaaggagaaagtggaccatcaggtcctgccggtcccactggtgctagaggtgcacctggtgatagaggtgaacctggtccaccagggcctgctggatttgctggtccaccaggagcagatggacaaccaggagcaaaaggtgagcctggagatgctggagctaaaggagatgcaggtcctcctggaccagctggacctgctggaccacctggaccaattggaaatgttggtgctccaggagctaaaggggcaagaggatctgctggtcctcctggagcaactgggttccctggagcagcaggaagagttggtcctcctggaccttctggaaacgctggacctcctggtccaccaggacctgctggaaaggaaggaggaaagggtccaagaggcgaaactggaccagcaggtagaccaggagaggttggaccacctggaccacctggtcccgctggagagaaaggatctcctggagctgatggaccagcaggtgctccaggcactccaggcccacaaggaattgctggtcaaaggggagttgttggattgcctgggcaaagaggagagaggggatttcctggtcttcctggtccatcaggtgaacctggaaaacaaggtccatctggagctagtggtgagaggggccctccaggaccaatgggcccacctggacttgctggacctcctggagagtccggtagagaaggggctccaggtgctgaaggatcaccaggaagggatggatctcctggagccaagggggatagaggagaaacaggtccagcagggcctcctggtgcaccaggtgcacctggtgctcctggtccagttggacctgcaggtaaatctggtgatcgtggagaaactggtccagctggacctgctggacctgttggaccagtgggtgctcgtggacctgctggtccacagggaccaagaggagataaaggtgagactggtgagcaaggtgatagaggaattaaaggacataggggtttttctggcttacagggtcctccaggtccaccaggatctccaggagaacaaggtccatcaggagctagtggaccagcagggccaaggggacctcctggttctgctggtgcaccaggtaaagatgggcttaacggattgcctggacctataggtcctccaggtccaagaggaagaactggtgatgctggtcctgttggaccaccaggtccacctggaccaccagggccacctggacctccatctgcaggatttgatttttctttccttccacaaccaccacaagaaaaggctcacgatggtggaaggtattatagggcaggctctggtgaagggcgtggaagtcttcttacatgtgaggatgttgaagaaaatccaggaccacaatatgatggaaagggtgttggattgggtccaggtccaatgggattgatgggccctagaggtcctcctggagctgctggagctcctggaccacaaggattccagggcccagctggtgaacctggagaaccgggacaaacaggaccagctggtgctagaggtccagctggtcctccaggaaaagctggagaagatggccatcctggtaaaccaggtaggccaggagaaagaggtgttgtgggtccacagggagctaggggatttcctggtactcctgggttgcctggattcaagggaattaggggtcataatggtcttgatggtcttaaaggacaaccaggagctcctggtgttaaaggagaacctggagcacctggtgaaaatggtactccaggtcaaacaggtgcaagaggattgccaggagaaaggggtagagtgggagcaccaggtcctgctggagctagaggttcagatggaagtgtgggacctgtgggacctgcaggaccaattggatcagctggtccacctggatttccaggtgccccaggtccaaagggagaaattggagctgttggaaatgcgggcccagcaggcccagctggacctagaggtgaggttggtctaccaggtctgtcaggaccagtgggccctccaggaaatcctggtgcaaatgggcttacaggagctaagggagcagctggattgcctggtgttgctggggcaccaggtcttcctggtccaagaggtattccaggaccagtaggtgctgcaggagcaactggagctagaggtttggttggtgaaccaggaccagcaggctccaagggtgaatctggtaataagggagaacctggttctgctggaccacaaggaccaccaggaccatcaggagaagaaggtaagaggggtcctaacggagaggccggttctgcaggtccacctggaccacctggacttagaggatctccagggtctagaggtttacctggtgctgatggtagagctggagtgatgggtcctccaggttcaagaggagcatctggcccagcaggagttaggggaccaaatggtgatgctgggagaccaggtgaaccaggtcttatgggtcctagaggattgccaggttcaccaggaaatattggtccagctggaaaagaaggaccagttggacttcctggaattgatggtagaccaggtcctattggtcctgctggtgctagaggtgagccaggtaatatcggttttccaggaccaaagggaccaactggtgatccaggcaaaaatggtgataagggacatgctggactcgcaggagctagaggcgctccaggacctgatggaaataatggtgcccagggacctccaggaccacaaggtgttcaaggaggaaagggtgagcaaggacctccaggacctccaggttttcagggacttccaggaccatctggaccagcaggtgaggttggtaagccaggagaaaggggtttacatggtgaattcggtctgccaggaccagctggaccaaggggtgaaaggggtccaccaggagagtcaggtgctgctggaccaacaggaccaattggttcaagaggtccatctggacctccaggtcctgatggaaacaaaggtgaaccaggagttgtaggtgctgttggaactgctggtccttcaggcccaagcggacttccaggtgaaaggggtgctgctggtattcctggaggtaagggtgaaaaaggggagcctggtcttagaggtgagattggtaatccaggaagagatggggctagaggtgcaccaggagccgttggtgctcctggtcctgctggagctacaggagatagaggagaggcaggagctgctggtcctgctggaccagctggcccaagaggtagcccaggagaaagaggtgaagttggtccagctggtcctaatggatttgctggacctgctggtgctgctggtcagcctggagctaaaggggaaagaggagccaaaggacctaaaggagaaaatggagttgttgggcctacaggaccagtaggagcagcaggacctgctggtccaaatggaccaccaggaccagcaggatccagaggagatggtggtccaccaggaatgacaggttttcctggtgctgctggaagaacaggaccaccaggtccttcaggtatttctggtcctccaggtcctccaggaccagctggaaaggagggtttgagaggacctagaggtgatcaaggacctgtgggaagaacaggagaagttggagcagttggaccaccaggtttcgctggagaaaagggaccatctggcgaagctggaactgctggaccaccaggtacccctggacctcagggacttcttggagctcctggaattttgggacttcccggatctagaggagagaggggattgccaggcgttgctggagctgtgggtgagccaggtcctctcggaattgctggaccacctggtgcaaggggtccaccaggtgccgttgggtccccaggtgttaatggtgctccaggtgaggctggtagggatggaaatccaggtaatgatggaccacctggtagggatggacagcctggccataagggtgagcgtggatatccaggtaatattggaccagttggtgcagcaggggcaccaggaccacacggacctgttggtccagctggtaagcacggcaataggggagagactggtccttcaggacctgtgggtccggctggagcagttggacctagaggtccatcaggaccacaaggaattagaggtgataagggagaacccggggagaaaggaccaagaggattacctggattaaagggtcacaatggattacaaggattgcctggaattgctggacatcacggagatcaaggagcaccaggatcagttggaccggctggaccaagaggacctgcaggaccttctggacctgctggtaaagatggaagaactggacatcctggtacagttggacctgctggaattagaggtccacaaggtcatcaagggcctgccggtcctccaggaccaccaggaccaccagggcctccaggagtttctggcggtggatatgattttggttatgatggagatttttaccgtgcttga

(19) United States
(12) Patent Application Publication
(10) Pub. No.: US 2025/0236651 A1
(43) Pub. Date: Jul. 24, 2025

(54) Recombinant Spider Silk-Reinforced Collagen Proteins Produced in Plants and the Use Thereof

(71) Applicant: SWIFTPHARMA BV, Gent (BE)

(72) Inventor: Jeroen Hofenk, Eindhoven (NL)

(21) Appl. No.: 18/853,919

(22) PCT Filed: Apr. 4, 2023
(86) PCT No.: PCT/EP2023/058733

§ 371 Date: Oct. 3, 2024

(30) Foreign Application Priority Data
Apr. 4, 2022 (EP) 22166454.3

(51) Int. Cl. C07K 14/435; C07K 1/16; C07K 1/34; C07K 14/78; C12N 1/20; C12N 15/62; C12R 1/01

(52) U.S. Cl. CPC C07K 14/43518; C07K 1/16; C07K 1/34; C07K 14/78; C12N 1/20; C12N 15/62; C07K 2319/02; C07K 2319/08; C12R 2001/01

(57) ABSTRACT

The invention described herein relates to a novel non-naturally occurring, elastomeric animal-free recombinant fusion biopolymers produced in plants through transient expression. More specifically, the present invention describes polynucleotides encoding fusion proteins of a non-human scleroprotein with a human collagen, wherein said fusion protein is capable of forming hydroxylated triple helix fibers. In particular fusion proteins of a Spidroin like protein with a human collagen. More in particular, eithera Spidroin-I/Collagen Type-I fusion protein, capable of forming hydroxylated triple helix fibers, or a Fibroin-III/Collagen Type-I fusion protein, capable of forming hydroxylated triple helix fibers. The present invention has improved properties (e.g., thermostability, young's modulus, cell adhesion, degradability, and the like) versus that of native Collagen Type-I. Also described are methods for use thereof, such as the use of electrospun scaffolds which are particularly well suited for biomedical or cosmetic applications as defined in the claims.

The invention described herein relates to a novel non-naturally occurring, elastomeric animal-free recombinant fusion biopolymers produced in plants through transient expression. More specifically, the present invention describes polynucleotides encoding fusion proteins of a non-human scleroprotein with a human collagen, wherein said fusion protein is capable of forming hydroxylated triple helix fibers. In particular fusion proteins of a Spidroin like protein with a human collagen. More in particular, eithera Spidroin-I/Collagen Type-I fusion protein, capable of forming hydroxylated triple helix fibers, or a Fibroin-Ill/Collagen Type-I fusion protein, capable of forming hydroxylated triple helix fibers. The present invention has improved properties (e.g., thermostability, young's modulus, cell adhesion, degradability, and the like) versus that of native Collagen Type-1. Also described are methods for use thereof, such as the use of electrospun scaffolds which are particularly well suited for biomedical or cosmetic applications as defined in the claims.

### BACKGROUND OF THE PRIOR ART

The extracellular matrix (ECM) is the non-cellular component present within all tissues and organs, and provides not only essential physical scaffolding for the cellular constituents but also initiates crucial biochemical and biomechanical cues that are required for tissue morphogenesis, differentiation and homeostasis [1].

Moreover, the ECM is a highly dynamic structure that is constantly being remodeled, either enzymatically or non-enzymatically, and its molecular components are subjected to a myriad of post-translational modifications. Through these physical and biochemical characteristics the ECM generates the biochemical and mechanical properties of each organ, such as its tensile and compressive strength and elasticity, and also mediates protection by a buffering action that maintains extracellular homeostasis and water retention. In addition, the ECM directs essential morphological organization and physiological function by binding growth factors (GFs) and interacting with cell-surface receptors to elicit signal transduction and regulate gene transcription. The biochemical and biomechanical, protective and organizational properties of the ECM in a given tissue can vary tremendously from one tissue to another (e.g. lungs versus skin versus bone) and even within one tissue (e.g. renal cortex versus renal medulla), as well as from one physiological state to another (normal versus cancerous).

The ECM is composed of two main classes of macromolecules: proteoglycans (PGs) and fibrous proteins [2, 3]. The main fibrous ECM proteins are collagens, elastins, and fibronectins [4]. PGs fill the majority of the extracellular interstitial space within the tissue in the form of a hydrated gel [2]. PGs have a wide variety of functions that reflect their unique buffering, hydration, binding and force-resistance properties. For example, in the kidney glomerular basement membrane, perlecan has a role in glomerular filtration [5, 6]. By contrast, in ductal epithelial tissues, decorin, biglycan and lumican associate with collagen fibers to generate a molecular structure within the ECM that is essential for mechanical buffering and hydration and that, by binding growth factors (GFs), provides an easy, enzymatically accessible repository for these factors.

Collagen is the most abundant fibrous protein within the interstitial ECM and constitutes up to 30% of the total protein mass of a multicellular animal. Collagens, which constitute the main structural element of the ECM, provide tensile strength, regulate cell adhesion, support chemotaxis and migration, and direct tissue development [7]. The bulk of interstitial collagen is transcribed and secreted by fibroblasts that either reside in the stroma or are recruited to it from neighboring tissues [8].

By exerting tension on the matrix, fibroblasts are able to organize collagen fibrils into sheets and cables and, thus, can dramatically influence the alignment of collagen fibers. Although within a given tissue, collagen fibers are generally a heterogeneous mix of different types, one type of collagen usually predominates.

The structure of collagen is a triple helix in which three polypeptide strands together form a helical coil. The individual polypeptide strands are composed of repeating triplet amino acid sequences designated as GLY-X-Y. X and Y can be any amino acid and the third amino acid is glycine. The amino acids proline and hydroxyproline are found in high concentrations in collagen. The most common triplet is proline-hydroxyproline-glycine (Gly-Pro-Hyp) accounting for approximately 10.5% of the triplets in collagen.

Collagen associates with elastin, another major ECM fiber. Elastin fibers provide recoil to tissues that undergo repeated stretch. Importantly, elastin stretch is crucially limited by tight association with collagen fibrils [9]. Secreted tropoelastin (the precursor of elastin) molecules assemble into fibers and become highly crosslinked to one another via their lysine residues by members of the lysyl oxidase (LOX) enzyme family, which include LOX and LOXL [10]. Elastin fibers are covered by glycoprotein microfibrils, mainly fibrillins, which are also essential for the integrity of the elastin fiber [9].

A third fibrous protein, fibronectin (FN) is intimately involved in directing the organization of the interstitial ECM and, additionally, has a crucial role in mediating cell attachment and function. FN can be stretched several times over its resting length by cellular traction forces [11]. Such force-dependent unfolding of FN exposes cryptic integrin-binding sites within the molecule that result in pleiotropic changes in cellular behavior and implicate FN as an extracellular mechano-regulator.

Like FN, other ECM proteins such as tenascin exert pleiotropic effects on cellular behavior, including the promotion of fibroblast migration during wound healing [12, 13]. Indeed, levels of tenascins C and W are elevated in the stroma of some transformed tissues where they can inhibit the interaction between syndecan4 and FN to promote tumor growth and metastasis [13].

Both collagen, elastin, and fibronectin are considered being biodegradable biopolymers, which are naturally occurring biomolecules with the highest degree of biocompatibility. In other words they are polymeric biomolecules. These also include other animal protein-based biopolymers such as wool, silk, gelatin, and polysaccharides such as cellulose, starch, carbohydrate polymers produced by bacteria and fungi. Biopolymers, especially the carbohydrate origin, have been found very promising for biomedical application in various forms because of their low toxicity, low antigenicity, high bio-activity, biodegradability, stability, processability to complicated shapes with appropriate porosity to support cell growth, mechanical properties, and renewable nature.

Biopolymers are generated from renewable sources and are easily biodegradable because of the oxygen and nitrogen atoms found in their structural backbone. Biodegradation converts them to CO², water, biomass, humid matter, and other natural substances. Biopolymers are thus naturally recycled by biological processes.

The bioactive properties such as antimicrobial, immune-modulatory, cell proliferative and angiogenic nature of the polymers create a microenvironment favorable for the healing process and provide a plethora of applications in pharmaceutical, medical, and cosmetic applications. One of the major advantageous properties of biopolymers though, is their ability to absorb large volumes of water when in the dry state and donate water when hydrated.

Both collagen and elastin are typically isolated from natural sources, such as bovine, porcine, or poultry hide, and for collagen also from cartilage, or bones. Bones are usually dried, defatted, crushed, and demineralized to extract collagen, while hide and cartilage are usually minced and digested with proteolytic enzymes. As collagen and elastin are resistant to most proteolytic enzymes, this procedure conveniently serves to remove most of the contaminating protein found with collagen or elastin.

Daniels et al U.S. Pat. No. 3,949,073, disclosed the preparation of soluble collagen by dissolving tissue in aqueous acid, followed by enzymatic digestion. The resulting atelopeptide collagen is soluble, and substantially less immunogenic than unmodified collagen. It may be injected into suitable locations of a subject with a fibril-formation promoter (described as a polymerization promoter in the patent) to form fibrous collagen implants in situ, for augmenting hard or soft tissue. This material is now commercially available from Collagen Corporation (Palo Alto, Calif.) under the trademark Zyderm® collagen implant.

Luck et al U.S. Pat. No. 4,488,911, disclosed a method for preparing collagen in solution (CIS), wherein native collagen is extracted from animal tissue in dilute aqueous acid, followed by digestion with an enzyme such as pepsin, trypsin, or Pronase®. The enzyme digestion removes the telopeptide portions of the collagen molecules, providing “atelopeptide” collagen in solution. The atelopeptide CIS so produced is substantially non-immunogenic, and is also substantially non-cross-linked due to loss of the primary crosslinking regions. The CIS may then be precipitated by dialysis in a moderate shear environment to produce collagen fibers which resemble native collagen fibers. The precipitated, reconstituted fibers may additionally be crosslinked using a chemical agent (for example aldehydes such as formaldehyde and glutaraldehyde), or using heat or radiation. The resulting products are suitable for use in medical implants due to their biocompatibility and reduced immunogenicity.

Wallace et al U.S. Pat. No. 4,424,208, disclosed an improved collagen formulation suitable for use in soft tissue augmentation. Wallace's formulation comprises reconstituted fibrillar atelopeptide collagen (for example, Zyderm® collagen) in combination with particulate, crosslinked atelopeptide collagen dispersed in an aqueous medium. The addition of particulate crosslinked collagen improves the implant's persistence, or ability to resist shrinkage following implantation.

Smestad et al U.S. Pat. No. 4,582,640, disclosed a glutaraldehyde crosslinked atelopeptide CIS preparation (GAX) suitable for use in medical implants. The collagen is crosslinked under conditions favoring intrafiber bonding rather than interfiber bonding, and provides a product with higher persistence than noncross-linked atelopeptide collagen, and is commercially available from Collagen Corporation under the trademark Zyplast® Implant. Nguyen et al U.S. Pat. No. 4,642,117, disclosed a method for reducing the viscosity of atelopeptide CIS by mechanical shearing. Reconstituted collagen fibers are passed through a fine-mesh screen until viscosity is reduced to a practical level for injection.

Nathan et al U.S. Pat. No. 4,563,350, disclosed osteoinductive bone repair compositions comprising an osteoinductive factor, at least 5% nonreconstituted (afibrillar) collagen, and the remainder reconstituted collagen and/or mineral powder (e.g., hydroxyapatite). CIS may be used for the nonreconstituted collagen, and Zyderm® collagen implant (ZCI) is preferred for the reconstituted collagen component. The material is implanted in bone defects or fractures to speed ingrowth of osteoclasts and promote new bone growth.

Chu U.S. Pat. No. 4,557,764, disclosed a “second nucleation” collagen precipitate which exhibits a desirable malleability and putty-like consistency. Collagen is provided in solution (e.g., at 2-4 mg/mL), and a “first nucleation product” is precipitated by rapid titration and centrifugation. The remaining supernatant (containing the bulk of the original collagen) is then decanted and allowed to stand overnight. The precipitated second nucleation product is collected by centrifugation.

Chu U.S. Pat. No. 4,689,399, disclosed a collagen membrane preparation, which is prepared by compressing and drying a collagen gel. The resulting product has high tensile strength.

J. A. M. Ramshaw et al, Anal Biochem (1984) 141:361-65, and PCT application WO87/04078 disclosed the precipitation of bovine collagen (types I, II, and Ill) from aqueous PEG solutions, where there is no binding between collagen and PEG.

Werner U.S. Pat. No. 4,357,274, disclosed a method for improving the durability of sclero protein (e.g., brain meninges) by soaking the degreased tissue in H2 O2 or PEG for several hours prior to lyophilizing. The resulting modified whole tissue exhibits increased persistence.

Hiroyoshi U.S. Pat. No. 4,678,468, disclosed the preparation of polysiloxane polymers having an interpenetrating network of water-soluble polymer dispersed within. The water-soluble polymer may be a collagen derivative, and the polymer may additionally include heparin. The polymers are shaped into artificial blood vessel grafts, and are designed to prevent clotting.

Other patents disclose the use of collagen preparations with bone fragments or minerals. For example, Miyata et al U.S. Pat. No. 4,314,380 disclosed a bone implant prepared by baking animal bone segments, and soaking the baked segments in a solution of atelopeptide collagen.

Deibig et al U.S. Pat. No. 4,192,021 disclosed an implant material which comprises powdered calcium phosphate in a pasty formulation with a biodegradable polymer (which may be collagen).

There are several references in the art to proteins modified by covalent conjugation to polymers, to alter the solubility, antigenicity and biological clearance of the protein. For example, U.S. Pat. No. 4,261,973 disclosed the conjugation of several allergens to PEG or PPG (polypropylene glycol) to reduce the proteins' immunogenicity. U.S. Pat. No. 4,301,144 disclosed the conjugation of hemoglobin with PEG and other polymers to increase the protein's oxygen carrying capability.

EPO 98,110 disclosed coupling an enzyme or interferon to a polyoxyethylene-polyoxypropylene (POE-POP) block polymer increases the protein's half-life in serum. U.S. Pat. No. 4,179,337 disclosed conjugating hydrophilic enzymes and insulin to PEG or PPG reduce immunogenicity.

Davis et al, Lancet (1981) 2:281-83 disclosed the enzyme uricase modified by conjugation with PEG to provide uric acid metabolism in serum having a long half-life and low immunogenicity.

Nishida et al, J Pharm Pharmacol (1984) 36.:354-55 disclosed PEG-uricase conjugates administered orally to chickens, demonstrating decreased serum levels of uric acid. Inada et al, Biochem & Biophys Res Comm (1984) 122:845-50 disclosed lipoprotein lipase conjugation with PEG to render it soluble in organic solvents.

M. Chvapil et al, J Biomed Mater Res (1969) 3:315-32 disclosed a composition prepared from collagen sponge and a crosslinked ethylene glycol monomethacrylate-ethylene glycol dimethacrylate hydrogel. The collagen sponge was prepared by lyophilizing an aqueous mixture of bovine hide collagen and methylglyoxal (a tanning agent). The sponge-hydrogel composition was prepared by polymerizing ethylene glycol monomethacrylate and ethylene glycol dimethacrylate in the sponge.

Kenton W. Gregory, U.S. Pat. No. 6,667,051B1 disclosed a method for joining at least one layer of pressed biomaterial to a tissue substrate, comprising: I) providing the pressed biomaterial consisting essentially of elastin or tropoelastin materials; II) applying a biodegradable cyanoacrylate adhesive to either one of the material and the tissue; and Ill) adhering the pressed biomaterial to the tissue and forming a substantially water-tight engagement therebetween.

Kenton W. Gregory et al, Pat. No. AU712868B2 disclosed elastin and elastin-based biomaterials and to methods of using same in tissue repair and replacement. The invention further relates to methods of securing the elastin and elastin-based biomaterials to existing tissue.

Nikolay Ouzounov et al, U.S. Pat. No. U.S. Ser. No. 11/041,015B2 disclosed non-naturally occurring full-length and truncated collagen molecules and full-length and truncated elastin molecules and their production, being recombinantly expressed in bacterial cells.

Véronique-Residence Sainte Madeleine Gruber et al, Pat. No. EP0951537B1 disclosed the production of recombinant collagens by plants, in particular by stable expression, and in particular homocatalar type I collagen [α1 (I) 3], as well as their uses.

Klaus During, Pat. No. CA2336064A1 disclosed a method for the production of a fibrous protein, comprising the following steps: (a) expression of a precursor fibrous protein in a plant cell and (b) incubation of the precursor fibrous protein with a protein processing the latter. The invention also relates to the plant cells used for this purpose and to the fibrous proteins produced according to the inventive method.

Oded Shoseyov et al, Pat. No. U.S. Pat. No. 9,783,816B2 disclosed a method of producing at least one type of a collagen alpha chain in a manner enabling accumulation of the collagen alpha chain in a subcellular compartment devoid of endogenous P4H activity, thereby producing the collagen in the plant.

Kirivikko et al (WO 93/07889) described the expression of procollagens humans (types I and II). 50 An example reports the expression of the chain alpha-1 or alpha-2 procollagen I or II in mammalian cells HT1080. A Another example describes the expression of the alpha and beta chains of prolyl-4-hydroxylase in Sf9 insect cells. Suggested examples for the expression of collagen and recombinant P4H are given for yeasts S. cerevisiae and P. pastoris, without demonstration of result.

While the mechanical properties and very useful biological agents of collagen, elastin, and fibronectin are recognized without ambiguity, the use of this protein is put back into question because of possible risks of contamination by unconventional infectious agents. Indeed, if the risks posed by bacterial contaminations or viruses can be perfectly controlled, it is not the same for those associated with prion-type agents.

These infectious agents that seem to be protein-like involved in the development of encephalopathies animal degenerative (scrapie of sheep, bovine spongiform encephalopathy) and human of Creutzfeld-Jacob, Gertstmann-Straussler syndrome, kuru). The period of their possible expression is such formal controls are difficult to achieve. These risks have already virtually blocked any marketing of human collagen and the provisions regulations made with regard to collagens concerned animals complicate purification processes and increase their cost.

In view of these difficulties and in the face of a radical deterioration in the image of mammalian collagen, one solution is the production of recombinant collagen which can be easily purified in a system which is not likely to cause pathogenic risks for humans and whose industrial cost is not prohibitive. The inventors have therefore developed the present invention e.g. a recombinant spidroin/collagen fusion protein transiently produced in plants.

Animal cells are, a priori, more adapted to the expression of mammalian genes. The enzymatic equipment that carries out the post-translational maturation however is different from a tissue, from one organ or species to another. For example, it has been reported that the post-translational maturation of a plasma protein may be different when it is obtained from human blood or when it is produced by a recombinant cell like cells of Chinese hamster ovaries or in the milk of an animal transgenic. In addition, low levels of expression obtained with mammalian cells involve in vitro cultures in very large volumes at high costs. The production of recombinant proteins in the milk of transgenic animals (mice, sheep, and cows) reduces the costs of production and overcome problems of expression level. However, there are still problems of ethics and contaminations viral and subviral (prions).

For these reasons, there has been an urgent need in developing novel biodegradable biopolymers that have a wider pleiotropic anti-inflammatory action, and improved toxicity, antigenicity, bio-activity, biodegradability, stability, processability, mechanical properties and renewable nature. Therefore, there is still the need to produce such biodegradable biopolymers with the aforementioned capabilities, obtained from a low-cost and highly productive process, in a highly purified state which served as the inspiration to develop present invention.

Consequential to its essential role as a mechanical support and affinity regulator in extracellular matrices, both collagen and silk constitutes a highly sought after scaffolding material for regeneration and healing applications. However, substantiated concerns have been raised with regard to quality and safety of animal tissue-extracted collagen or spider- or silkworm-derived silk, particularly in relation to its immunogenicity, risk of disease transmission and overall quality and consistency. In parallel, contamination with undesirable cellular factors can significantly impair its bioactivity, vis-a-vis its impact on cell recruitment, proliferation and differentiation. Overall, the shortcomings of animal- and cadaver-derived collagens arising from their source diversity and recycled nature are fully overcome by the present invention.

### DESCRIPTION OF THE INVENTION

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Notwithstanding the exemplary embodiments described hereinbelow, is the present invention only limited by the attached claims. The attached claims are hereby explicitly incorporated in this detailed description, in which each claim, and each combination of claims as allowed for by the dependency structure defined by the claims, forms a separate embodiment of the present invention.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof.

In the present described invention, various specific details are presented. Embodiments of the present invention can be carried out without these specific details. Furthermore, well-known features, elements and/or steps are not necessarily described in detail for the sake of clarity and conciseness of the present disclosure.

Reference throughout this specification to “one embodiment” or “an embodiment” or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention 50 requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The non-essential improvement and adjustment made by the method disclosed by the invention should still be within the protection scope of the invention. Meanwhile, raw materials being used are not always described in detail. The raw materials may be commercially available products. The process steps or preparation methods that are not always described in detail are process steps or preparation methods which are known by those skilled in the art.

The raw materials used to obtain the present invention are commercially available products; the process steps or the preparation method which are also not always described in detail, are process steps or preparation methods which are all known by those skilled in the art.

As used herein the term “about” refers to ±10%.

The term “consisting of means “including and limited to”.

The term “consisting essentially of” means that the composition, process or structure may contain additional components, steps and/or parts, but only if the additional components, steps and/or parts meet the basic and novel properties of the claimed composition and therefor do not materially alter the basic and novel characteristics of the claimed composition and/or method or structure.

As used herein, the singular forms “a”, “an” and “the” include plural references unless the context clearly indicates otherwise. For example, the term “a compound” or “at least one compound” can encompass a variety of compounds, including mixtures thereof which can be used in foods, cosmetics, pharmaceuticals, industrial products, medical products, laboratory culture growth media, and many other applications.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first 50 indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In the description provided herein, numerous specific details are set forth. However, it is understood that embodiments of the invention may be practiced without these specific details. In other instances, well-known methods, structures and techniques have not been shown in detail in order not to obscure an understanding of this description.

It is an object of the present invention to provide a novel fusion biopolymers consisting of 2 moieties being: I) a recombinant non-human scleroprotein, and a II) human recombinant Collagen, in particular human Collagen Type I. The non-human scleroprotein, is preferably not a non-human Collagen. In an embodiment the non-human scleroprotein is selected from a keratin, elastin, fibrin or spidroin; more in particular from a fibrin or spidroin, also commonly referred to as spidroin-like proteins.

In one embodiment the present invention provides a novel fusion biopolymers consisting of 2 moieties being: I) recombinant a Fibroin-Ill, and a II) human recombinant Collagen Type I. Other combinations (such as, but not limited to, Collagen-Type II to Type XXVIII, any chain, or any derivative thereof, are also within the scope of this present invention.

In one embodiment the present invention provides a novel fusion biopolymers consisting of 2 moieties being: I) recombinant spidroin-1, and a II) human recombinant Collagen Type I. Other combinations (such as, but not limited to, Collagen-Type II to Type XXVIII, any chain, or any derivative thereof, Spidroin-II, minor ampullate spidroins, flagelliform spidroin, any type of fibroin, or any derivative thereof), are also within the scope of this present invention.

It is an object of the present invention to provide the novel fusion biopolymers as herein disclosed in large scale in which proteins can readily be manipulated to polymerize into fibers at wish.

The term “collagen” or “collagen-like” as used herein refers to a monomeric polypeptide that can form a quaternary structure with one or more collagen or collagen-like polypeptides. The quaternary structure of natural collagen is a triple helix typically composed of three polypeptides. Of the three polypeptides that form natural collagen, two are usually identical and are designated as the alpha chain. The third polypeptide is designated as the beta chain. Thus a typical natural collagen can be designated as AAB, wherein the collagen is composed of two Colα1(1) chains and one Colα2(1) chain. The term “procollagen” as used herein refers to polypeptides produced by cells that can be processed to naturally occurring collagen. Preferably, the collagen chain expressed in this present invention, is an alpha l and 2 chain of type I collagen, although other types may be used as well. Examples include other Fibril-forming collagens (types II, III, V, and XI), network forming collagens (types IV, VIII, and X), collagens associated with fibril surfaces (types IX, XII, and XIV), collagens which occur as transmembrane proteins (types XIII 50 and XVII), or form 11-nm periodic beaded filaments (type VI). For further description please see Hulmes, 2002. The expressed collagen alpha I and II chains can be encoded by any polynucleotide sequences derived from any mammal. Preferably, the amino acid sequences encoding collagen alpha I and II chains are human and are set forth by SEQ NOs: 1 and 2. Their respective nucleotide sequences are set forth by SEQ NOs: 3 and 4. Preferably, the nucleotide sequences have been codon optimized for chloroplast expression in N. benthamiana.

The term “Spidroins” as used herein refers to the main proteins in spider silk. Different types of spider silk contain different spidroins, all of which are members of a single protein family. The two most ubiquitous types of spidroins are the major ampullate silk proteins (MaSp) used in the construction of dragline silk. Dragline silk fiber is made up of two types of spidroins being: the Major Ampulate Spidroin-1 (MaSp1) and Spidroin-2 (MaSp2) proteins.

The term “fibroin” as used herein refers to insoluble scleroproteins comprising the filaments of raw silk fiber obtained from spiders or silkworms. Preferably, fibroin is obtained from spider species by means of recombinant technology. Alternatively fibroin may be even well obtained from silkworm species, for example but not limited to Bombyx mori and other moth genera such as Antheraea, Cricula, Samia and Gonometa, by means of obtaining fibroin from a solution containing dissolved silkworm silk, or by recombinant technology. Preferably, both Spidroin and Fibroin are obtained from spider species including, but not limited to: Arachnura higginsi, Araneus circulissparsus, Araneus diadematus, Argiope picta, Banded Garden Spider (Argiope trifasciata), Batik Golden Web Spider (Nephila antipodiana), Beccari's Tent Spider (Cyrtophora beccarii), Bird-dropping Spider (Celaenia excavata), Black-and-White Spiny Spider (Gasteracantha kuhlii), Black-and-yellow Garden Spider (Argiope aurantia), Bolas Spider (Ordgarius furcatus), Bolas Spiders—Magnificent Spider (Ordgarius magnificus), Brown Sailor Spider (Neoscona nautica), Brown-Legged Spider (Neoscona rufofemorata), Capped Black-Headed Spider (Zygiella calyptrata), Common Garden Spider (Parawixia dehaani), Common Orb Weaver (Neoscona oxancensis), Crab-like Spiny Orb Weaver (Gasteracantha cancriformis (elipsoides)), Curved Spiny Spider (Gasteracantha arcuata), Cyrtophora moluccensis, Cyrtophora parnasia, Dolophones conifera, Dolophones turrigera, Doria's Spiny Spider (Gasteracantha doriae), Double-Spotted Spiny Spider (Gasteracantha mammosa), Double-Tailed Tent Spider (Cyrtophora exanthematica), Aculeperia ceropegia, Eriophora pustulosa, Flat Anepsion (Anepsion depressium), Four-spined Jewel Spider (Gasteracantha quadrispinosa), Garden Orb Web Spider (Eriophora transmarina), Giant Lichen Orbweaver (Araneus bicentenarius), Golden Web Spider (Nephila maculata), Hasselt's Spiny Spider (Gasteracantha hasseltii), Tegenaria atrica, Heurodes turrita, Island Cyclosa Spider (Cyclosa insulana), Jewel or Spiny Spider (Astracantha minax), Kidney Garden Spider (Araneus mitificus), Laglaise's Garden Spider (Eriovixia laglaisei), Long-Bellied Cyclosa Spider (Cyclosa bifida), Malabar Spider (Nephilengys malabarensis), Multi-Coloured St Andrew's Cross Spider (Argiope versicolor), Ornamental Tree-Trunk Spider (Herennia ornatissima), Oval St. Andrew's Cross Spider (Argiope aemula), Red Tent Spider (Cyrtophora unicolor), Russian Tent Spider (Cyrtophora hirta), Saint Andrew's Cross Spider (Argiope keyserlingi), Scarlet Acusilas (Acusilas coccineus), Silver Argiope (Argiope argentata), Spinybacked Orbweaver (Gasteracantha cancriformis), Spotted Orbweaver (Neoscona domiciliorum), St. Andrews Cross (Argiope aetheria), St. Andrew's Cross Spider (Argiope Keyserlingi), Tree-Stump Spider (Poltys illepidus), Triangular Spider (Arkys clavatus), Triangular Spider (Arkys lancearius), Two-spined Spider (Poecilopachys australasia), Nephila species, e.g. Nephila clavipes, Nephila senegalensis, and Nephila madagascariensis. Most preferred, the Spidroin proteins are derived from Nephila clavipes, and the Fibroin proteins from Araneus diadematus. Not surprisingly, Fibroin proteins are also considered Spidroin-like analogues [42]. The amino acid sequence of Spidroin-I expressed in this present invention is 50 set forth by SEQ NO: 5. Its respective nucleotide sequence is set forth by SEQ NO: 6. Preferably the nucleotide sequence has been codon optimized for chloroplast expression in N. benthamiana. Preferably the Fibroin sequence expressed in this present invention, is Fibroin-III and its amino acid sequence is set forth by SEQ NO: 7. Its nucleotide sequence is set forth by SEQ NO: 8. Preferably, the nucleotide sequence has been optimized for chloroplast expression in N. benthamiana.

According to one aspect of the present invention, a method of producing a Collagen protein in a plant or an isolated plant cell comprising expressing in the plant or the isolated plant cell at least one type of a Collagen Alpha Chain and exogenous Proline 4-hydroxylase (P4H) in a manner enabling accumulation of the at least one type of the Collagen Alpha Chain and the exogenous P4H in a subcellular compartment devoid of endogenous P4H activity, thereby producing a collagen protein in the plant is defined in the claims. In prior described art, an attempt to produce human collagens that rely on the hydroxylation machinery naturally present in plants resulted in collagen that is poor in proline hydroxylation which has been described by Merle et al., 2002 [14]. Such collagen melts or loses its triple helical structure at temperatures below 30° C. Co-expression of collagen and prolyl-hydroxylase results with stable hydroxylated collagen that is biologically relevant for applications at body temperatures [14]. As is used herein, the phrase “subcellular compartment devoid of endogenous P4H activity” refers to any compartmentalized region of the cell which does not include plant P4H or an enzyme having plant-like P4H activity. Examples of such subcellular compartments include the vacuole, apoplast and cytoplasm as well as organelles such as the chloroplast, mitochondria and the like. Accumulation of the expressed collagen chain in a subcellular compartment devoid of endogenous P4H activity can be effected via any one of several approaches. For example, the expressed collagen chain can include a signal sequence for targeting the expressed protein to a subcellular compartment such as the apoplast or more preferably, the chloroplast or other organelles such as the mitochondria. Examples of suitable signal sequences include the chloroplast transit peptide (included in Uniprot entry G5DBJ0, amino acids 1-40) and the Mitochondrion transit peptide (included in Uniprot entry Q9ZP06, amino acids 1-22). The Examples section which follows provides additional examples of suitable signal sequences as well as guidelines for employing such signal sequences in expression of collagen chains in plant cells. Alternatively, the sequence of the collagen chain can be modified in a way which alters the cellular localization of collagen when expressed in plants. As is mentioned hereinabove, the endoplasmic reticulum (ER) of plants includes a P4H which is incapable of correctly hydroxylating collagen chains. Collagen alpha chains natively include an ER targeting sequence which directs expressed collagen into the ER where it is post-translationally modified (including incorrect hydroxylation). Thus, removal of the ER targeting sequence will lead to cytoplasmic accumulation of collagen chains which are devoid of post translational modification including any hydroxylations.

As is also mentioned hereinabove, hydroxylation of alpha chains is required for assembly of a stable type I collagen. Full collagen proline hydroxylation also significantly raises the melting temperature by stabilizing the collagen triple helix, a process that is well understood by those known in the art. Since alpha chains expressed by transient expression in the wildtype plant of the present invention accumulate in a compartment devoid of endogenous P4H activity, such chains must normally be isolated from the plant, plant tissue or cell and correctly hydroxylated using an in-vitro technique which can be achieved by the method of Torre-Blanco A, Alvizouri A. [15]. However, such method is cumbersome and costly to achieve the desired effect. To overcome this limitation, the method of the present invention also transiently co-expresses P4H which is capable of correctly hydroxylating the collagen alpha chain(s) (i.e., hydroxylating only 50 the proline (Y) position of the Gly-X-Y triplets). P4H is an enzyme composed of two subunits, alpha and beta, and both are needed to form an active catalytic enzyme [16]. Mammalian prolyl 4-hydroxylase is an alpha-2/beta-2 tetramer [17]. The 59-kDa alpha subunit contains the substrate-binding domain and the enzymic active site [18]. Humans and most other vertebrates have three isoforms of the alpha subunit, isoform alpha-1 is the most prevalent. The pair of alpha subunits can be any of the three isoforms [19]. The 55-kDa beta subunit is protein disulphide isomerase (PDI), which has additional functions in collagen formation. As part of P4H it retains the tetramer in the ER lumen and maintains the otherwise insoluble alpha subunit in an active form. In prior art, the inventors of patent no° EP2816117B1, Collagen producing plants and methods of generating and using same, used an exogenous human P4H to generate a stable transformant (e.g., transgenic) plant, capable of producing human P4H with the objective to correctly hydroxylate only the proline (Y) position of the Gly-X-Y triplets. However, tetrameric human P4H is inhibited by poly(L-proline) by extensine molecules that are substrates of plant prolyl-4-hydroxylase which are rich in Ser-(Pro)₄-Ser-Pro-Ser-(Pro)₄ sequences and thus could inhibit P4H of mammalian origin. To overcome this limitation, the inventor of the present invention, uses an alternative approach and generated a chimeric alpha/beta dimer with the same specific activity as native human P4H but without the inhibition potential by poly-(L-proline). The resulting chimeric P4H enzyme consists of an alpha subunit of a Dictyostelium discoideum (Slime mold) (UniprotKB: Q86KR9) and the beta subunit of a Bos taurus (Bovine) P4H (Uniprot: P05307). According to further features in the described preferred embodiments the exogenous chimeric P4H includes a signal peptide for targeting to the chloroplast and is devoid of an ER targeting or retention sequence. The amino acid sequences encoding for the chimeric P4H enzyme expressed in this present invention are set forth by SEQ NOs: 9 for the P4H subunit alpha chain, and 10 for the P4H subunit beta chain, respectively. Their respective nucleotide sequences are set forth by SEQ NOs: 11 and 12. Preferably, the nucleotide sequences have been optimized for chloroplast expression in N. benthamiana.

In mammalians, the enzymes Lysyl hydroxylase, galactosyltransferase and glucosyltransferase sequentially modify lysyl residues in specific positions to hydroxylysyl, galactosylhydroxylysyl, and glucosylgalactosyl hydroxylysyl residues in collagen. However, the multi-functional enzyme Lysin Hydroxylase 3 (LH3) as set forth in Genbank No. 060568, is the only human enzyme is capable of converting collagen lysines into 1,2-glucosylgalactosyl-5-hydroxylysines through three consecutive reactions: hydroxylation of collagen lysines (LH activity), N-linked conjugation of galactose to hydroxylysines (GT activity), and conjugation of glucose to galactosyl-5-hydroxylysines (GGT activity). These enzymes are known to act together with prolyl hydroxylases, respectively introducing hydroxylations of lysine and proline residues on collagens in the endoplasmic reticulum (ER), prior to the formation of triple-helical assemblies [20]. According to further features in the described preferred embodiments the exogenous LH3 includes a signal peptide for targeting to the chloroplast and is devoid of an ER targeting or retention sequence. The amino acid sequence encoding for the LH3 enzyme expressed in this present invention is set forth by SEQ NO: 13. Its respective nucleotide sequence is set forth by SEQ NO: 14. Preferably, the nucleotide sequence has been optimized for chloroplast expression in N. benthamiana.

According to one aspect of the present invention, a method of producing a fibrillar Spidroin-I/Collagen Type-I fusion protein is provided comprising transiently co-expressing two vectors in which vector 1 expresses A) a spidroin-I chain, B) a Collagen Type-I alpha-I chain, and C) a Collagen Type-I Alpha-II chain, wherein transient expression is configured such that the Spidroin-I chain and the Collagen Alpha-I and Alpha-II chains are each capable of accumulating in a subcellular compartment devoid of both endogenous P4H and LH activity. Such compartment 50 is preferably the chloroplast and is functionalized by using a transit signal leading to the chloroplast. Vector 2 expresses A) the aforementioned chimeric P4H and B) LH3, both of which are capable of accumulating in the subcellular compartment devoid of both endogenous P4H and LH activity. Both vectors are preferably targeted to a the chloroplast by introducing a chloroplast transit peptide at the N-terminal of the respective gene constructs. Such transit peptide is preferably, but not limited to, the chloroplastic Protein Chaperone-Like Protein of POR1. The respective genes assembled in both expression vectors are separated by introducing so-called “2A self-cleaving peptides” which can induce ribosomal skipping during translation of a protein in a cell, thus making it possible to generate multiple separated sequences expressed within a single transcript. Such fusion protein is termed “SPIDICOL1” from here on in this present invention. The amino acid sequence encoding for the SPIDICOL1 fusion protein in vector 1 is set forth by SEQ NO: 15. Its respective nucleotide sequence is set forth by SEQ NO: 16. Preferably, the nucleotide sequence has been optimized for chloroplast expression in N. benthamiana. The amino acid sequences encoding for the P4H/LH3 proteins in vector 2 are set forth by SEQ NO: 17. Their respective nucleotide sequence is set forth by SEQ NO: 18. Preferably, the nucleotide sequence has been optimized for chloroplast expression in N. benthamiana.

According to one aspect of the present invention, a method of producing a fibrillar Fibroin-Ill/Collagen Type-I fusion protein is provided comprising transiently co-expressing two vectors in which vector 1 expresses A) a Fibroin-Ill chain, B) a Collagen Type-I alpha-I chain, and C) a Collagen Type-I Alpha-II chain, wherein transient expression is configured such that the Fibroin-Ill chain and the Collagen Alpha-I and Alpha-II chains are each capable of accumulating in a subcellular compartment devoid of both endogenous P4H and LH activity. Such compartment is preferably the chloroplast and is functionalized by using a transit signal leading to the chloroplast. Vector 2 expresses A) the aforementioned chimeric P4H and B) LH3, both of which are capable of accumulating in the subcellular compartment devoid of both endogenous P4H and LH activity. Both vectors are preferably targeted to a the chloroplast by introducing a chloroplast transit peptide at the N-terminal of the respective gene constructs. Such transit peptide is preferably, but not limited to, the chloroplastic Protein Chaperone-Like Protein of POR1. The respective genes assembled in both expression vectors are separated by introducing so-called “2A self-cleaving peptides” which can induce ribosomal skipping during translation of a protein in a cell, thus making it possible to generate multiple separated sequences expressed within a single transcript. Such fusion protein is termed “FIB3COL1” from here on in this present invention. The amino acid sequence encoding for the FIB3COL1 fusion protein in vector 1 is set forth by SEQ NO: 19. Its respective nucleotide sequence is set forth by SEQ NO: 20. Preferably, the nucleotide sequence has been optimized for chloroplast expression in N. benthamiana. The amino acid sequences encoding for the P4H/LH3 proteins in vector 2 are set forth by SEQ NO: 17. Their respective nucleotide sequence is set forth by SEQ NO: 18. Preferably, the nucleotide sequence has been optimized for chloroplast expression in N. benthamiana.

According to still further features in the described preferred embodiments the plant is selected from the group consisting of Tobacco, Maize, Alfalfa, Rice, Potato, Soybean, Tomato, Wheat, Barley, Canola, beets, sunflower, and Cotton, more preferably Nicotiana benthamiana in which the portion of the is leaves, seeds, roots, tubers or stems, more preferably the leaves.

Plant: is generally understood as meaning any single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and 50 dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Most preferred are plants which are not used for food or feed purpose such as Arabidopsis thaliana, or preferably Nicotiana tabacum, or most preferably Nicotiana benthamiana. Alternatively, plants which are used for food or feed purposes can be used as well, such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (eggplant), and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

According to still further features in the described preferred embodiments the plant is subjected to a stress condition. Such stress conditions are selected from the group consisting of drought, salinity, injury, cold and spraying with stress inducing compounds and/or compounds known in the art to increase endogenous ascorbate (Vitamin C) levels. As both P4H and LH3 enzymes are long known to suffer oxidative inactivation during catalysis, and the cofactor ascorbate (vitamin C) is required to reactivate the enzyme by reducing its iron center from Fe(Ill) to Fe(II), it may be beneficial to administer Vitamin C by means of biofortification [21].

According to another aspect of the present invention there is provided a method of transiently expressing or isolated plant cell capable of accumulating a collagen alpha chain having a hydroxylation pattern identical to that produced when the collagen alpha chain is expressed in human cells.

The term “transient expression” or “transient gene expression” as used herein refers to the temporary expression of genes that are expressed for a short time after a nucleic acid, most frequently plasmid DNA encoding an expression cassette, has been introduced into eukaryotic 50 cells. Transient expression should result in a time-limited use of transferred nucleic acids, since any long-term expression would be called “stable expression”. The use of transient expression in a plant cell or a plant according to the invention makes it possible to produce high yields compatible with a commercial exploitation. In the case of transient expression, harvesting of the plant biomass takes place during peak expression of the recombinant protein, e.g., typically 5 to 9 days after transfection. Transient expression of the aforementioned recombinant fusion proteins in Nicotiana Benthamiana plants may advantageously enable a high throughput platform to produce the compound at industrial scale and/or at a low cost. However, embodiments of the present invention are not necessarily limited thereto, e.g. any other suitable expression host may equally be used, including but not limited to, bacteria, yeast, insect, mammalian, or other plant expression systems.

The term “fluorescent protein” is a protein that is commonly used in genetic engineering technologies used as a reporter of expression of an exogenous polynucleotide. The protein when exposed to ultraviolet or blue light fluoresces and emits a bright visible light. Proteins that emit green light is green fluorescent protein (GFP) and proteins that emit red light is red fluorescent protein (RFP).

The term “gene” as used herein refers to a polynucleotide that encodes a specific protein, and which may refer to the coding region alone or may include regulatory sequences preceding (5′ non-coding sequences) and following (3′ non-coding sequences) the coding sequence.

The term “host cell” is a cell that is programmed to express an introduced exogenous polynucleotide, preferably this host cell is the chloroplast subcellular compartment of Nicotiana benthamiana (N. benthamiana).

The term “non-naturally occurring” as used herein refers to collagen, Spidroin, or Fibroin, that is not normally found in nature. The non-naturally occurring collagen, Spidroin and Fibroin moieties are recombinantly prepared. The non-naturally occurring Collagen, Spidroin, or Fibroin protein is a recombinant Collagen, recombinant Spidroin, or recombinant Fibroin.

The term “signal peptide” refers to an amino acid sequence that recruits the host cell's cellular machinery to transport an expressed protein to a particular location or cellular organelle of the host cell. Preferably the target peptide sequence is located on the C-terminal end of the amino acid structure of the protein. Preferably the signal peptide is a transit peptide, functionalizing targeting to the chloroplast.

According to another aspect of the present invention, the resulting Spidroin/Collagen or Fibroin/Collagen fusion proteins are purified after extraction from the plant or plant cells that express it. In order to facilitate their purification, the fusion proteins may be expressed in fusion with tags (His6, GST, MBP, FLAG etc.) which will preferably be located in the N- or C-terminal position of the mature protein.

The general methods of growing plants, as well as methods for introducing expression vectors into plant tissue, are available to those skilled in the art. They are varied and depend on the selected plant. In general, this method comprises a first step of cultivating the plant, aeroponic or hydroponic, preferably free float culture, and under LED lighting. After this first step, in particular five weeks of hydroponic culture on free floats, agroinfiltration plants is carried out under vacuum, by agrobacteria comprising a DNA fragment coding for the aforementioned Spidroin/Collagen or Fibroin/Collagen fusion proteins according to the invention. This step of agroinfiltration can be implemented by any means to evacuate. Preferably, in the method used 50 according to the invention, it is carried out under vacuum by the Venturi effect. Agrobacterium refers to a soil-borne, Gram-negative, rod-shaped phytopathogenic bacterium which causes crown gall. The term “Agrobacterium” includes, but is not limited to, the strains Agrobacterium tumefaciens (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine, etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

After agroinfiltration, the plants are typically further cultured for 5 to 9 days. Finally, the protein is extracted and purified using industry-standard methods known in the art.

The term “expression vector” or “vector” as used herein refers to a nucleic acid assembly which is capable of directing the transient expression of the exogenous gene. The expression vector may include a promoter which is operably linked to the exogenous gene, restriction endonuclease sites, nucleic acids that encode one or more selection markers, and other nucleic acids useful in the practice of recombinant technologies. Preferably, the expression vector used in step a) comprises: prokaryotic DNA elements encoding an origin of bacterial replication and an antibiotic resistance gene; at least one heterologous nucleotide sequence coding for the aforementioned fusion proteins according to the invention operatively linked to a strong promoter, preferably a 35S promoter; an expression cassette for the expression of a silencing inhibitor, preferably p19; and DNA elements that control the processing of transcripts, such as termination/polyadenylation sequences, preferably the Tnos sequence. Numerous plant functional expression promoters and enhancers which can be either tissue specific, developmentally specific, constitutive or inducible can be utilized in conjunction with the constructs of the present invention. As used herein in the specification and in the claims section that follows the phrase “plant promoter” or “promoter” includes a promoter which can direct gene expression in plant cells (including DNA containing organelles, more specifically the protoplast). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters. The plant promoter employed can be a constitutive promoter, a tissue specific promoter, an inducible promoter or a chimeric promoter. Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter. Examples of tissue specific promoters include, without being limited to, bean phaseolin storage protein promoter, DLEC promoter, PHS promoter, zein storage protein promoter, conglutin gamma promoter from soybean, AT2S1 gene promoter, ACT11 actin promoter from Arabidopsis, napA promoter from Brassica napus and potato patatin gene promoter.

It will be appreciated that constructs including two expressible inserts (for example a Spidroin chain and a Collagen Type I Alpha I and/or Alpha II chain, or a P4H and a LH3 sequence) preferably include an individual promoter for each insert, or alternatively such constructs can express a single transcript chimera including both insert sequences from a single promoter. In 50 such a case, the chimeric transcript includes a “self-cleaving” 2A sequence (e.g., a 2A sequence is used to express two proteins from a single promoter in an expression construct) between the two insert sequences such that the downstream insert can be translated therefrom. Preferably T2A is used, coding for (GSG)EGRGSLLTCEDVEENPGP, for which (GSG) residues can be added to the 5′ end of the peptide to improve cleavage efficiency. Other 2A sequences such as but not limited to:

(GSG)ATNFSLLKQAGDVEENPGP,
(GSG)QCTNYALLKLAGDVESNPGP
(GSG)VKQTLNFDLLKLAGDVESNPGP

Such use of 2A sequences may circumvent the limitations of commonly known Internal Ribosome Entry Site (IRES) sequences. These elements are quite large (500-600 bp) and may take up precious space in viral transfer vectors (with limited packaging capacity). Additionally, it may not be feasible to express more than two genes at a time using IRES elements. Further, scientists have reported lower expression of the downstream cistron due to factors such as the experimental cell type and the specific genes cloned into the vector [22].

Collagen and silk are extensively used in the biomedical, regenerative medicine, food and cosmetics industry. Thus, although for both collagen and silk fiber components and modifying enzymes expressed by plants find utility in industrial synthesis of collagen and silk, complete collagen and/or silk production in plants is preferred for its simplicity and cost effectiveness.

The present invention successfully addresses the shortcomings of the presently known collagen configurations by providing a plant capable of expressing correctly hydroxylated Spidroin/Collagen or Fibroin/Collagen fusion proteins with improved properties (e.g., thermostability, young's modulus, cell adhesion, and the like) versus that of native human collagen. The resulting Spidroin/Collagen or Fibroin/Collagen fusion proteins thus obtained can be used in biomedical applications, cosmetics, esthetics, but not limited thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

A large quantity of biochemically modified, active recombinant photolyase fusion protein, highly purified in N. Benthamiana plants, can be obtained. To facilitate rapid, simple purification of the recombinant photolyase fusion protein, a 6×His tag, also known as polyhistidine tag, His6 tag and/or hexa histidine tag, may be attached to the N-terminus of the protein. We have demonstrated that the recombinant fusion proteins are biologically active and show improved functionality when compared to native heterotrimeric Collagen Type I.

N. benthamiana is a particularly suitable bioreactor for the transient expression of recombinant protein in a manufacturing setting. The small ornamental plant has a high leaf to stem ratio and is very prolific in hydroponic culture. N. benthamiana tolerates the transfection vectors and delivers maximum synthesis of heterologous proteins in 5-7 days after transfection. Scale-up of this bioreactor is a matter of growing more plants, not re-engineering processes.

Plants have all the eukaryotic cell machinery to accurately produce human and animal proteins. Thus, the bioreactor is the individual plant. Plants are well suited to express complex proteins such as monoclonal antibodies, and minimize risk by not supporting growth of human or animal pathogens.

For all agroinfiltration experiments, discussed hereinbelow, 5-week-old N. benthamiana plants were used.

N. Benthamiana seeds were grown in a greenhouse. Seedling and germination of N. benthamiana plants were carried out under LED illumination in a 16/8 h light/dark cycle, 7 days/week. Red and blue diodes were selected that match the action spectrum of photosynthesis (25% blue and 75% red). Other wavelengths were not productive. The LED's were focused on the plants. Plants grown to usable maturity 20% faster in this system as compared to other commercial solutions. All seeds were germinated using rockwool growing medium at 26.6° C., using an ebb and flow hydroponic, well known in the art.

### Example One: Spidroin-I/Collagen Type-I Fusion Protein

As aforementioned, two separate vectors are constructed. One of which carries the Spidroin-1/Collagen Type-I construct, the other one carrying the P4H/LH3 construct. Both vectors are designed to target a subcellular compartment known to be devoid of endogenous P4H or LH activity.

The inventor constructed a fundamental set of Golden Gate cloning-compatible modular vectors which comprise the expression cassette and acceptor backbones named GGC-TC1 and GGC-TC2.

The acceptor backbone is a binary T-DNA vector suitable for transient expression in plants and is designed to possess the geminiviral replicon system, capable of producing circular DNA replicons for high-level multiple protein expression.

### 1) Spidroin-I/Collagen Type I Tricistronic Construct:

For the biosynthesis of the genes of interest; Spidroin-1 (Uniprot: P19837, entry version N° 69, 2017-03-15), Collagen Type-I Alpha-I without the signal peptide (1-22) and without the N-terminal propeptide (23-161) and C-terminal propeptide (1219-1464) (Uniprot: P02452, entry version N° 22, 2017-05-10), Collagen Type-I Alpha-II without the signal peptide (1-22) and without the N-terminal propeptide (23-79) and C-terminal propeptide (1120-1366) (Uniprot: P08123, entry version N° 202, 2017-05-10), an A2 sequence GSGEGRGSLLTCEDVEENPGP placed between the Spidroin-1, Collagen Type-I Alpha-1, and Collagen Type-I Alpha-II chain, a chloroplast transit peptide (included in Uniprot entry G5DBJ0, amino acids 1-40, entry version N° 10, 2017-11-22) placed at the N-terminus, and a 6× HIS Tag were used as a template.

2) P4H Alpha-Beta Chimeric/LH3 Tricistronic Vector Construct: For the biosynthesis of the genes of interest; a chimeric P4H enzyme comprising: an alpha subunit (Uniprot: Q86KR9, entry version N° 81, 2017-06-07) and a beta subunit (Uniprot: P05307, entry version N° 141, 2017-05-10) sequence without its native signal peptide (1-20), an A2 sequence GSG EGRGSLLTCEDVEENPGP placed between the alpha subunit and beta subunit, a LH3 sequence without its native signal peptide (1-24) (Uniprot: 060568, entry version N° 165, 2017-09-27), an A2 sequence GSGEGRGSLLTCEDVEENPGP placed between LH3 and the PH4 beta subunit sequence, and a chloroplast transit peptide (included in Uniprot entry G5DBJ0, amino acids 1-40, entry version N° 10, 2017-11-22) placed at the N-terminus, were used as a template.

Using the Golden Gate cloning approach, a T2A-linked tricistronic vector, whereby three transgenes encoding I) Spidroin-I and II) Collagen Type-I proteins (termed SPIDICOL1) were combinatorially placed along the expression cassette in the binary vector. The P4H alpha-beta chimeric/LH3 constructs also comprises 3 genes (e.g., P4H alpha subunit, P4H beta subunit, and LH3, termed P4HLH3) and were combinatorially placed along the expression cassette.

Using the assembly protocol described in the Golden Gate modular cloning system (Weber et al., 2011) [23], so-called “level-0” modular vectors containing parts of the expression cassette, such as promoter (Pro), T2A signals (T2A), coding sequences (CDS), and terminator (Ter), were constructed. All level-0 modules were flanked by inward-facing Bsal restriction enzyme sites and fusion sites (5 bp-overhangs) to allow directional linear assembly in a Pro-CDS1-T2A-CDS2-T2A-CDS3-Ter (SPIDICOL1) and Pro-CDS1-T2A-CDS2-T2A-CDS3-Ter (P4HLH3) orientation, resulting in a T2A-linked tricistronic constructs. In order to construct Pro, T2A, and Ter modules, sequences of CmYLCV promoter, and AtHSP 3′ UTR were retrieved from publications of the prior art being: Stavolone et al. (2003) [24], Nagaya et al. (2010) [25], and Liu et al. (2017) [26], respectively.

As the aforementioned genes described above, employ tandem rare codons and could reduce the efficiency of translation or even disengage the translational machinery, the codon usage bias in N. benthamiana was used by upgrading the codon adoption index (CAI) from 0.70 to 0.91. The GC content and unfavorable peaks have been optimized to prolong the half-life of the mRNA. The Stem-Loop structures, which impact ribosomal binding and stability of mRNA, were broken. In addition, negative cis-acting sites were screened and successfully modified. Pro, T2A, CDS and Ter modules were harbored into a pLUG-Prime vector (iNtRON Biotechnology). Codon-optimized CDS modules, were prepared by PCR using primers carrying inward-facing Bsal sites and fusion sites.

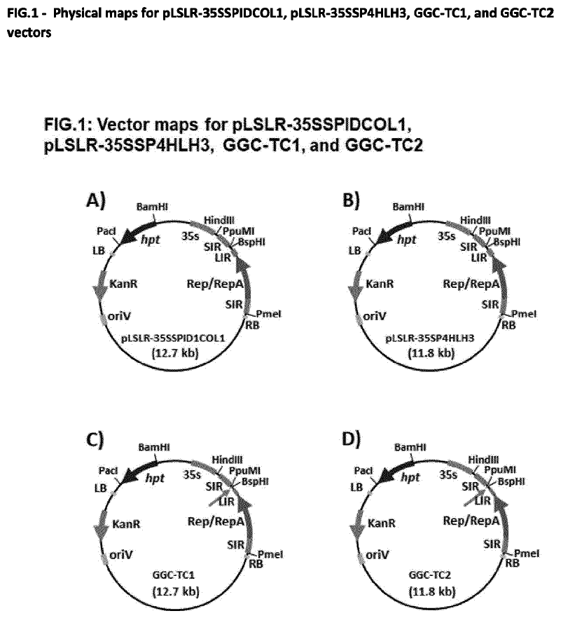

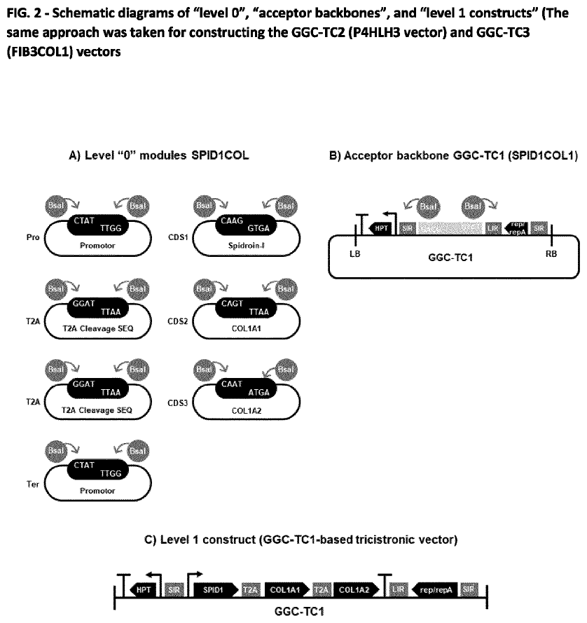

“Level 1” acceptor backbones were constructed based on a modified pLSLR vector (Baltes et al., 2014) [27](Addgene plasmid #51493). Firstly, CaMV 35S promoter flanked by UB11 intron was inserted into BamHI and Hindlll-digested pLSLR with a pCAMBIA1300 backbone, and the existing bi-directional cis-acting replication elements “LIR-SIR-LIR” (LIR=Long Intergenic Region, SIR=Short Intergenic Region) were placed in a “SIR-LIR-SIR” architecture. A benefit of this architecture and delivery mechanism is that the population of replicating viral genomes is both homogenous and predictable, consisting of the sequence between the origins within the duplicated SIRs. The resulting vectors were named pLSLR-35SSPID1COL1 and pLSLR-35SP4HLH3, respectively. A fragment flanked by two Bsal sites (5′-CTATGGAGACCGAGGTCTCGTAAG-3′) for Golden Gate cloning was then inserted into pLSLR-35SSPID1COL1 and pLSLR-35SP4HLH3. Cloning into PpuMI- and BspHI-digested pLSLR-35SSPID1COL1 and pLSLR-35SP4HLH3 formed GGC-TC1 and GGC-TC2, respectively. (See FIG. 1 for physical maps of resulting vectors) To construct T2A-linked tricistronic (GGC-BC1 and GGC-TC1) vectors in a Pro-CDS1-T2A-CDS2-T2A-CDS3-Ter orientation, level 0 modules were directionally assembled into the level 1 acceptor backbone using a single digestion-ligation procedure. An equal molar ratio of level 0 modules and level 1 acceptor was mixed with Bsal (Bsal-HFv2, New England Biolabs) and T4 ligase (Thermo Fisher). The reaction was carried out for 10 cycles of 5 min at 37° C. and 10 min at 16° C., followed by 5 min at 50° C. and 5 min at 80° C. Assembled level 1 constructs were amplified in Escherichia coli DH5a, and the subsequent plasmid recovery, restriction digestion, and sequencing procedures confirmed correct vector assembly. The resulting tricistronic vectors were transformed into agropine-type Agrobacterium tumefaciens EHA 105 and octopine-type Agrobacterium tumefaciens LBA4404, respectively by electroporation to carry out agroinfiltration experiments. The transformed cells were plated on LB agar medium containing 50 mg/ml Ampicillin (Sigma Aldrich). (See FIG. 2 for schematic diagrams of “level 0”, “acceptor backbones”, and “level 1 constructs”)

Agroinfiltration was used for transient expression in N. Benthamiana with A. tumefaciens. strains as previously described. 100 ml of transformed Agrobacterium frozen cells stock was inoculated in 5 ml LB broth (Thermo Fisher) and supplemented with 50 μg/ml rifampicin and 50 μg/ml kanamycin. Overnight, the culture was incubated at 28° C., shaking at 220 rpm. 500 ml was used to inoculate 50 ml of LB medium. The cultural cells were incubated at 28° C. shaking at 220 rpm until the culture had reached an O.D.600=0.6. The cells were harvested by centrifugation at 6000 rpm and resuspended in 50 ml MES buffer (10 mM MES; pH 5.5, IOmM MgC). This mixture was incubated for 2.5 hours at room temperature with 120 mM acetosyringone and was added to the Agrobacterium suspension in infiltration buffer (Ix MS, 10 mM MES, 2.5% glucose).

For the effect of monosaccharide on induction of virulence genes, different 2% was added to the Agrobacterium suspension in the infiltration buffer (Ix MS, 10 mM MES, 200 mM acetosyringone). 5-weeks old N. benthamiana plants were infiltrated in a vacuum chamber by submerging N. Benthamiana plant aerial tissues in Agrobacterium suspension and applying a 50-400 mbar vacuum for 45 seconds. Once the vacuum was broken, infiltrated N. Benthamiana plants were removed from the vacuum chamber, thoroughly rinsed in water, and grown for 5-7 days under the same growth conditions used for pre-infiltration growth. To avoid any variability, the leaves and location on the leaf, comparably-sized leaves for each plant of similar age were agroinfiltrated for each experiment.

### Example Two: Fibroin-III/Collagen Type-I Fusion Protein

As aforementioned, two separate vectors are constructed. One of which carries the Fibroin-Ill/Collagen Type-I construct, the other one carrying the P4H/LH3 construct. Both vectors are designed to target a subcellular compartment known to be devoid of endogenous P4H or LH activity.

The inventor constructed a fundamental set of Golden Gate cloning-compatible modular vectors which comprise the expression cassette and acceptor backbones named GGC-TC3 and GGC-TC2. The acceptor backbone is a binary T-DNA vector suitable for transient expression in plants and is designed to possess the geminiviral replicon system, capable of producing circular DNA replicons for high-level multiple protein expression.

### 1) Fibroin-III/Collagen Type I Tricistronic Construct:

For the biosynthesis of the genes of interest; Fibroin-Ill (Uniprot: Q16987, entry version N° 44, 2017-08-30), Collagen Type-I Alpha-I without the signal peptide (1-22) and without the N-terminal propeptide (23-161) and C-terminal propeptide (1219-1464) (Uniprot: P02452, entry version N° 22, 2017-05-10), Collagen Type-I Alpha-II without the signal peptide (1-22) and without the N-terminal propeptide (23-79) and C-terminal propeptide (1120-1366) (Uniprot: 50 P08123, entry version N° 202, 2017-05-10), an A2 sequence GSGEGRGSLLTCEDVEENPGP placed between the Fibroin-Ill, Collagen Type-I Alpha-1, and Collagen Type-I Alpha-II chain, a chloroplast transit peptide (included in Uniprot entry G5DBJ0, amino acids 1-40, entry version N° 10, 2017-11-22) placed at the N-terminus, and a 6× HIS Tag were used as a template.

### 2) P4H Alpha-Beta Chimeric/LH3 Tricistronic Vector Construct:

For the biosynthesis of the genes of interest; a chimeric P4H enzyme comprising: an alpha subunit (Uniprot: Q86KR9, entry version N° 81, 2017-06-07) and a beta subunit (Uniprot: P05307, entry version N° 141, 2017-05-10) sequence without its native signal peptide (1-20), an A2 sequence GSG EGRGSLLTCEDVEENPGP placed between the alpha subunit and beta subunit, a LH3 sequence without its native signal peptide (1-24) (Uniprot: 060568, entry version N° 165, 2017-09-27), an A2 sequence GSGEGRGSLLTCEDVEENPGP places between LH3 and the PH4 beta subunit sequence, and a chloroplast transit peptide (included in Uniprot entry G5DBJ0, amino acids 1-40, entry version N° 10, 2017-11-22) placed at the N-terminus, were used as a template.

Using the Golden Gate cloning approach, a T2A-linked tricistronic vector, whereby three transgenes encoding I) Fibroin-Ill and II) Collagen Type-I proteins (termed FIB3COL1) were combinatorially placed along the expression cassette in the binary vector. The P4H alpha-beta chimeric/LH3 constructs also comprises 3 genes (e.g., P4H alpha subunit, P4H beta subunit, and LH3, termed P4HLH3) and were combinatorially placed along the expression cassette.

Using the assembly protocol described in the Golden Gate modular cloning system (Weber et al., 2011) [23], so-called “level-0” modular vectors containing parts of the expression cassette, such as promoter (Pro), T2A signals (T2A), coding sequences (CDS), and terminator (Ter), were constructed.

All level-0 modules were flanked by inward-facing Bsal restriction enzyme sites and fusion sites (5 bp-overhangs) to allow directional linear assembly in a Pro-CDS1-T2A-CDS2-T2A-CDS3-Ter (FIB3COL1) and Pro-CDS1-T2A-CDS2-T2A-CDS3-Ter (P4HLH3) orientation, resulting in T2A-linked tricistronic constructs. In order to construct Pro, T2A, and Ter modules, sequences of CmYLCV promoter, and AtHSP 3′ UTR were retrieved from publications of the prior art being: Stavolone et al. (2003) [24], Nagaya et al. (2010) [25], and Liu et al. (2017) [26], respectively.

As the aforementioned genes described above, employ tandem rare codons and could reduce the efficiency of translation or even disengage the translational machinery, the codon usage bias in N. benthamiana was used by upgrading the codon adoption index (CAI) from 0.70 to 0.91. The GC content and unfavorable peaks have been optimized to prolong the half-life of the mRNA. The Stem-Loop structures, which impact ribosomal binding and stability of mRNA, were broken. In addition, negative cis-acting sites were screened and successfully modified.

Pro, T2A, CDS and Ter modules were harbored into a pLUG-Prime vector (iNtRON Biotechnology). Codon-optimized CDS modules, were prepared by PCR using primers carrying inward-facing Bsal sites and fusion sites.

“Level 1” acceptor backbones were constructed based on a modified pLSLR vector (Baltes et al., 2014) [27](Addgene plasmid #51493). Firstly, CaMV 35S promoter flanked by UB11 intron was inserted into BamHI and Hindlll-digested pLSLR with a pCAMBIA1300 backbone, and the existing bi-directional cis-acting replication elements “LIR-SIR-LIR” (LIR=Long Intergenic Region, SIR=Short Intergenic Region) were placed in a “SIR-LIR-SIR” architecture. A benefit of this architecture and delivery mechanism is that the population of replicating viral genomes is both 50 homogenous and predictable, consisting of the sequence between the origins within the duplicated SIRs. The resulting vectors were named pLSLR-35SSPID1COL1 and pLSLR-35SP4HLH3, respectively. A fragment flanked by two Bsal sites (5′-CTATGGAGACCGAGGTCTCGTAAG-3′) for Golden Gate cloning was then inserted into pLSLR-35SFIB3COL1 and pLSLR-35SP4HLH3. Cloning into PpuMI- and BspHI-digested pLSLR-35SFIB3COL1 and pLSLR-35SP4HLH3 formed GGC-TC3 and GGC-TC2, respectively.

To construct T2A-linked tricistronic (GGC-TC3 and GGC-TC2) vectors in a Pro-CDS1-T2A-CDS2-T2A-CDS3-Ter orientation, level 0 modules were directionally assembled into the level 1 acceptor backbone using a single digestion-ligation procedure. An equal molar ratio of level 0 modules and level 1 acceptor was mixed with Bsal (Bsal-HFv2, New England Biolabs) and T4 ligase (Thermo Fisher). The reaction was carried out for 10 cycles of 5 min at 37° C. and 10 min at 16° C., followed by 5 min at 50° C. and 5 min at 80° C. Assembled level 1 constructs were amplified in Escherichia coli DH5a, and the subsequent plasmid recovery, restriction digestion, and sequencing procedures confirmed correct vector assembly. The resulting tricistronic vectors were transformed into agropine-type Agrobacterium tumefaciens EHA 105 and octopine-type Agrobacterium tumefaciens LBA4404, respectively by electroporation to carry out agroinfiltration experiments. The transformed cells were plated on LB agar medium containing 50 mg/ml Ampicillin (Sigma Aldrich).

Agroinfiltration was used for transient expression in N. Benthamiana with A. tumefaciens. strains as previously described. 100 ml of transformed Agrobacterium frozen cells stock was inoculated in 5 ml LB broth (Thermo Fisher Scientific) and supplemented with 50 μg/ml rifampicin and 50 μg/ml kanamycin. Overnight, the culture was incubated at 28° C., shaking at 220 rpm. 500 ml was used to inoculate 50 ml of LB medium. The cultural cells were incubated at 28° C. shaking at 220 rpm until the culture had reached an O.D.600=0.6. The cells were harvested by centrifugation at 6000 rpm and resuspended in 50 ml MES buffer (10 mM MES; pH 5.5, IOmM MgC). This mixture was incubated for 2.5 hours at room temperature with 120 mM acetosyringone and was added to the Agrobacterium suspension in infiltration buffer (Ix MS, 10 mM MES, 2.5% glucose). For the effect of monosaccharide on induction of virulence genes, different 2% was added to the Agrobacterium suspension in the infiltration buffer (Ix MS, 10 mM MES, 200 mM acetosyringone). 5-weeks old N. benthamiana plants were infiltrated in a vacuum chamber by submerging N. Benthamiana plant aerial tissues in Agrobacterium suspension and applying a 50-400 mbar vacuum for 45 seconds.

Once the vacuum was broken, infiltrated N. Benthamiana plants were removed from the vacuum chamber, thoroughly rinsed in water, and grown for 5-7 days under the same growth conditions used for pre-infiltration growth. To avoid any variability, the leaves and location on the leaf, comparably-sized leaves for each plant of similar age were agroinfiltrated for each experiment.

### Results

Extraction and purification of SPIDICOL1 and FIB3COL1 heterotrimeric fusion proteins For the extraction of both SPIDICOL1 and FIB3COL1 proteins, infiltrated N. benthamiana leaves (300 g for each protein) were harvested and grinded, and blended (in 3 intervals of 1 minute each) with 2.5 g of activated carbon and cold (4° C.) extraction buffer (100 mM Tris-HCl pH 8.0, 4 mM EDTA, 600 mM NaCl, 25 mM DL-Dithiothreitol (DTT), 0.5% NP40, 2% Poly-(Vinyl-Poly-Pyrolidone) (PVPP), 10% glycerol and 2×Roche EDTA-free Complete protease inhibitor cocktail (Roche Diagnostics, Germany) at a ratio of 2 ml per gram of leaves (fresh weight). During this 50 protocol, the temperatures were kept below 12° C. The resulting crude extracts were then filtered using Whatman No. 1 filter paper, followed by centrifugation of the filtered extract (15000 g for 30 min at 5° C.). The resulting supernatants were then collected, and together with 1 g/L activated carbon, CaCI 2 was added at a final concentration of 10 mM. Nonsoluble contaminants were then further removed by centrifugation (20000 g for 30 min at 15° C.).

Both SPIDICOL1 and FIB3COL1 in the recovered supernatants were precipitated by gradually adding crystalline NaCl to a final concentration of 2.85 M (20 min, at room temperature with constant stirring). The solutions were incubated in a cold room for 6h without stirring. Collection of the SPIDICOL1-containing and FIB3COL1-containing pellets were performed following centrifugation (22000 g for 2 h at 5° C.). The pellets were then resuspended in a 200 mL solution of 250 mM acetic acid+2 M NaCl for 5 min, using a magnetic stirrer, and then centrifuged (22000 g for 30 min at 5° C.). Supernatants were then discarded, and the pellets were resuspended in 200 mL of 0.5 M acetic acid (for 1 h at room temperature). Elimination of insoluble matter was performed by centrifugation (15000 g for 30 min at 15° C.). The resulting supernatants were passed through 3 layers Whatman No. 1 filter paper.

The resulting SPIDICOL1 and FIB3COL1 proteins were then precipitated by slowly adding NaCl to a final concentration of 3 M along with constant stirring for 25 min at room temperature. The solution was incubated in a cold room for 8 h at 4° C. and the SPIDICOL1 and FIB3COL1 proteins were collected following centrifugation (22000 g for 2.5 h at 5° C.). All remaining supernatant traces were removed. Pellet redissolving and SPIDICOL1 and FIB3COL1 precipitation steps were repeated as above in acetic acid and NaCl solutions, respectively. Following the incubation and collection of SPIDICOL1-containing and FIB3COL1-containing pellets, the samples were redissolved in 50 mL of 10 mM HCl by vigorously pipettation and vortexing for 5 min at room temperature. The solutions were transferred to dialysis bags (Thermo Fisher, MWCO 25000 Da) and dialyzed against 5 L of 10 mM HCl (for 3 h at 4° C.). An additional dialysis was performed. Both SPIDICOL1 and FIB3COL1 proteins were sterilized by filtering through a 0.2 p filter using 30 mL syringes. The SPIDICOL1 and FIB3COL1 proteins were then concentrated using Vivaspin PES 6 mL filtration tubes (Sartorius, MWCO 300000) before loading into Nickel-nitrilotriacetic (Ni-NTA) affinity resin (Amintra). Briefly, the column was washed with 10 column volumes of wash buffer (5 mM and 20 mM Imidazole, 20 mM Tris-HCl, 50 mM NaCl, pH 7.4, respectively) and eluted the recombinant protein with elution buffer (250 mM Imidazole, 20 mM Tris-HCl, 50 mM NaCl, pH 7.4). The purified SPIDICOL1 and FIB3COL1 protein samples were analyzed by SDS-PAGE, Southern blot analysis, Western blot analysis, and quantified by ELISA. The total soluble protein (TSP) in the plant crude extracts was estimated by using Bradford assay (Bio-Rad) by following manufacturer's instruction.

### Southern Blot Analysis

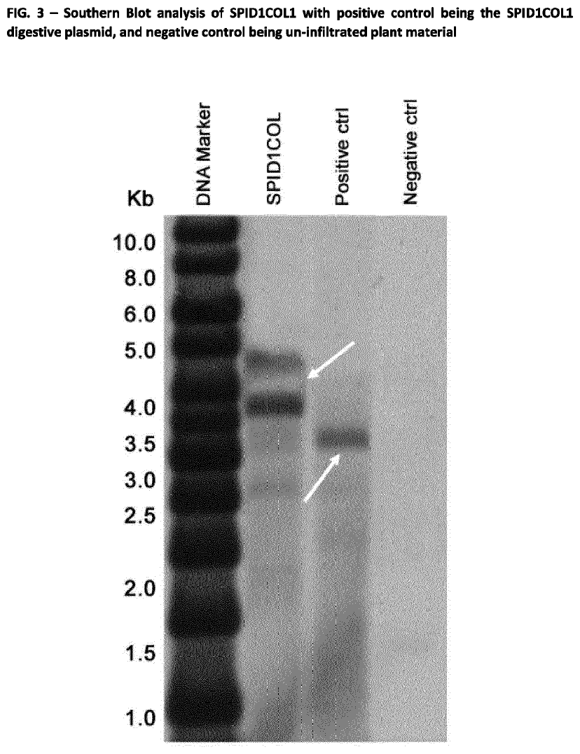

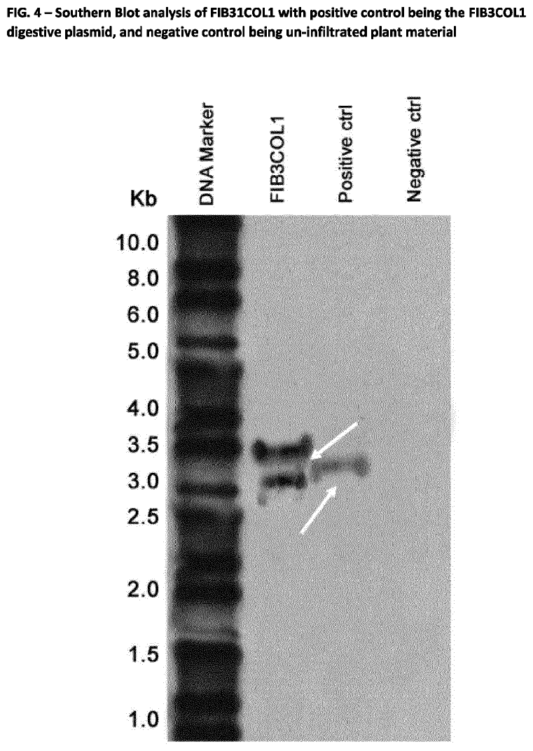

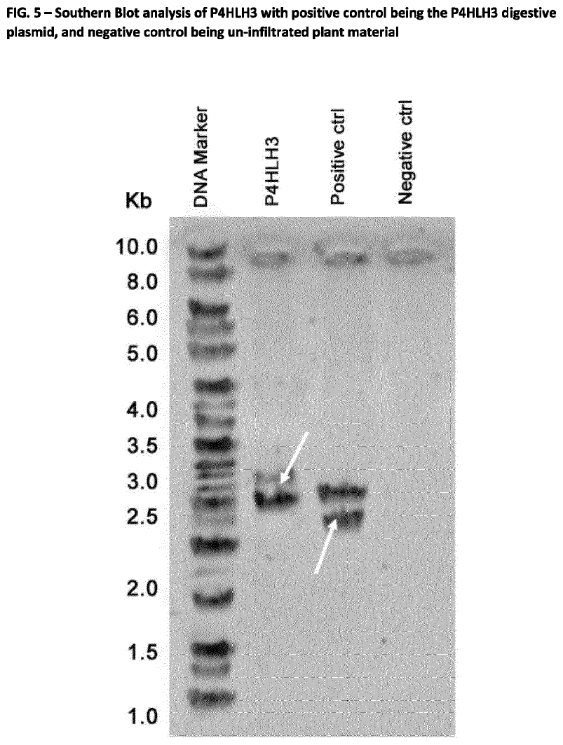

Genomic DNA from the agroinfiltrated leaves expressing SPIDICOL1, FIB3COL1 and P4HLH3, respectively, were extracted by DNeasy Plant DNA mini kit (Qiagen) and digested with both EcoRI/BgIII and subjected to Southern blot analysis. Results showed that both the synthetic SPIDICOL1, FIB3COL1, and the P4HLH3 open reading frames (ORF) were successfully transformed into N. benthamiana leaves after agroinfiltration. Labeling and detection were carried out using Biotin Deca Label DNA Labeling Kit, ThermoScientific and Biotin chromogenic Detection kit, ThermoScientific, respectively. The presence of amplified fragments with the expected sizes indicates that the genes were successfully transformed into N. benthamiana leaves via agroinfiltration. Higher molecular weight fragments were visualized due to partial digestion of some DNA of the samples. The digested recombinant GGC-TC1, GGC-TC2, and GGC-50 TC3 vectors were used as positive control and resulted in the same size band while the un-infiltrated leaves were used as negative control. (See FIGS. 3, 4 and 5 for Southern Blot results using SPIDICOL1 probe, FIB3COL1 probe and P4HLH3 probe for the total DNA of infiltrated tobacco leaves after digestion with EcoRI and BgIII)

### Detection of Chimeric Genes by RT-PCR

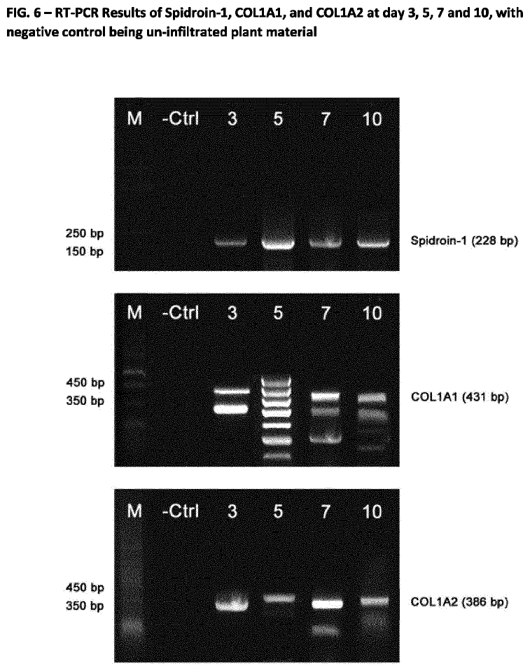

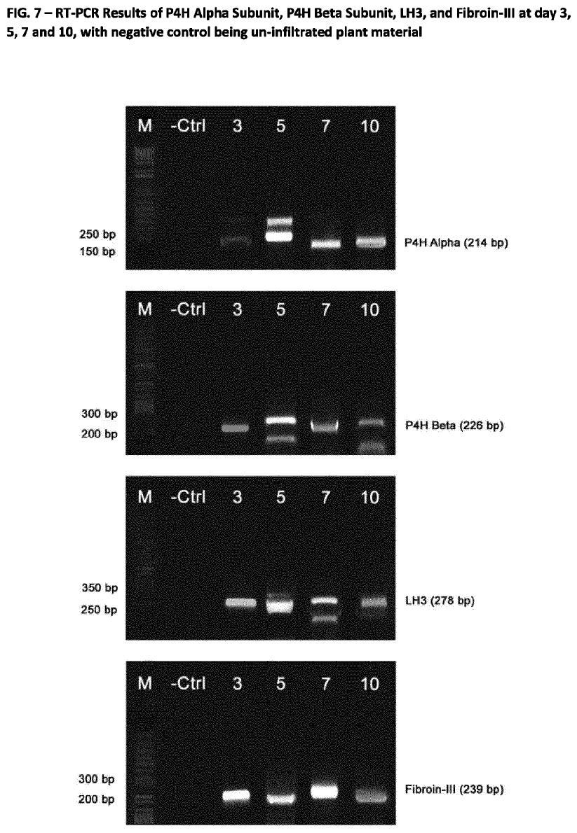

Transcription for both the respective Spidroin-1, Collagen Type I Alpha 1, Collagen Type I Alpha 11, Fibroin-3, P4H Alpha Subunit, P4H Beta Subunit, and LH3 genes was confirmed using Reverse-Transcription Polymerase Chain Reaction (RT-PCR). The extracted RNA samples from infiltrated N. benthamiana leaves were subjected to RT-PCR analysis using specific primers for each gene to amplify the core region of each gene. Total RNA was extracted using Illustra RNAspin mini kit (GE healthcare). Oligonucleotide pairs at the core region were designed to detect the presence of the respective genes at the core region; for Spidroin-1; TE-F: 5′-GGAGGACAAGGAGCTGGAG-3′, and TE-R: 5′-CTAGAAGCAGCAGCAGAAGC-3′, for Collagen Type-I Alpha-1; TE-F: 5′-ACCTATGGGACCTCCTGGAT-3′, and TE-R: 5′-GCAGGTCCAGTTTCTCCTCT-3′, for Collagen Type-I Alpha-II; TE-F: 5′-AGAACCTGGATCTGCTGGAC-3′, and TE-R: 5′-CCAGGAGGTCCCATTACTCC-3′, for P4H alpha subunit; TE-F: 5′-GCTGGAATGAATAAAGGAACTGA-3′, and TE-R: 5′-ATCTTCCTCCATTTAAATATACAGCTA-3′, for P4H beta subunit; TE-F: 5′-TCCTGCTTCTGCTGATAGAACT-3′, and TE-R: 5′-TCAGGTTCTTCAGCTTCTTCT-3′, for LH3; TE-F: 5′-TGTAGTACATGGAAATGGACCT-3′, and TE-R: 5′-GGAGGAGGTTGTCCTCCAG-3′, for Fibroin-Ill; TE-F: 5′-CTGCTGCTGGAGGATATGGA-3′, and TE-R: 5′-TCCTCCAGGTCCTTGTTGTC-3′. One step RT-PCR was carried out according to manufacturer instructions using SuperScript®111 with Platinum® Taq DNA Polymerase. The reactions resulted in the expected bp-fragments of the core region of the genes being; Spidroin-1 (228 bp-fragment), Collagen Alpha-I (431 bp-fragment), Collagen Alpha-lI (386 bp-fragment) P4H alpha subunit (214 bp-fragment), P4H beta subunit (226 bp-fragment), LH3 (278 bp-fragment), and Fibroin-Ill (239 bp-fragment). The RT-PCR amplified fragments of indicated that all the infiltrated leaves at day 3, 5, 7, and 10 clearly exhibited the transcription of the respective genes as shown in FIGS. 6 and 7, while un-infiltrated leaves showed negative results.

### Western Blot Analysis

Western blot was performed to confirm the production of both the chimeric SPIDICOL1 and FIB3COL1 proteins within plant's tissue. Total soluble proteins were extracted from infiltrated plants by grinding 500 mg of leaves in 0.5 mL 50 mM Tris-HCl (pH 7.5) enriched with 1× Roche EDTA-free Complete protease inhibitor cocktail (Roche Diagnostics, Germany). The crude extract was boiled for 5 minutes in 300 μL of 4× SDS Sample Loading Buffer (Sigma Aldrich: Tris-HCl: 0.2 M, DTT: 0.4 M, SDS: 277 mM, 8.0% (w/v), Bromophenol blue: 6 mM, Glycerol: 4.3 M) and centrifuged (12000 rpm for 7 min, at room temperature). Supernatant samples (25 μL) were separated on a 10% polyacrylamide gel (NuPAGE BIS-TRIS gel, Thermo Fisher) and proteins of interest were immunodetected using standard Western blot procedures. Detection of Spidroin-1, Collagen Type-I Alpha-I chain, Collagen Type-I Alpha-II chain, P4H alpha subunit, P4H beta subunit, LH3, and Fibroin-Ill was effected using a custom designed anti-Dictyostelium discoideum (Slime mold) P4H alpha subunit antibody, a custom designed anti-Bovine P4H beta subunit antibody, an anti-rabbit-LH antibody (LSBio), anti-rabbit polyclonal antibody to MASP (MASP1) (LSBio), anti-collagen type I antibody (OriGene Technologies) antibody, and a custom designed anti-rabbit polyclonal antibody to Fibroin-Ill. Broad range prestained protein marker were purchased from Thermo Fisher (PageRuler Prestained Protein Ladder, 30 to 240 kDa). As anticipated it showed no reactivity with un-infiltrated plants which were used as negative control. (See FIG. 8 for western blot results comparative over days 1, 3, 5, 7, 9, and 10 post-infiltration and leaf position top, middle, and base)

### Thermal Stability

To assess the thermal stability of the SPIDICOL1 and FIB3COL1 proteins, their sensitivity to either pepsin or a trypsin/chymotrypsin mixture was determined according to the method of P.

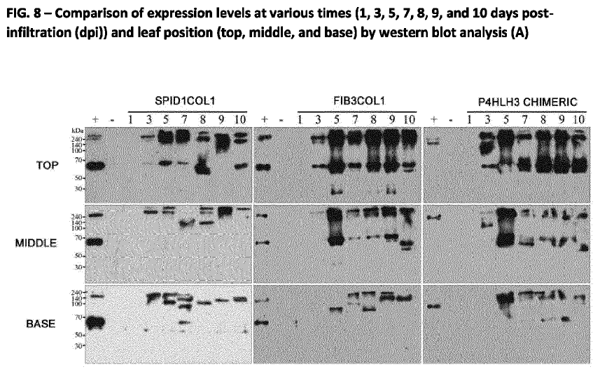

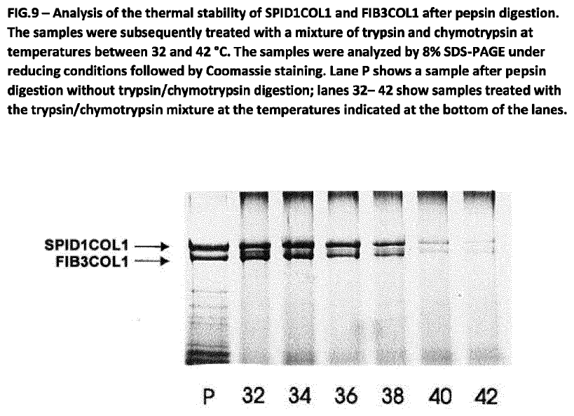

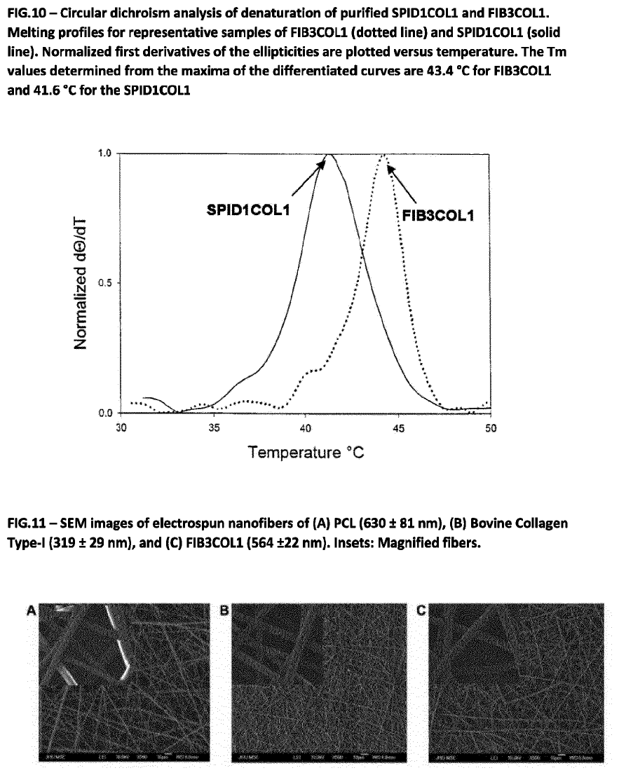

Bruckner (1981) [28]. Using a temperature range between 32° C. and 42° C. the study showed that both purified SPIDICOL1 and FIB3COL1 were resistant to pepsin up to 39.4 and 39.8° C., respectively (50% degradation point as measured by scanning of SPIDICOL1 and FIB3COL1 bands after PAGE) (FIG. 9). To obtain more accurate data on the thermal stability of the resulting SPIDICOL1 and FIB3COL1 proteins, circular dichroism (CD) spectra were performed. CD measurements of 451 g/mL SPIDICOL1 or 451 g/mL FIB3COL1, prepared in 10 mM HCl were performed using a Jasco J-810 Circular Dichroism Spectropolarimeter (Jasco) in a UV Fused Quartz Cuvette with 10 mm Path Length (CV10Q7A, Thorlabs). The cuvette was filled with 1 mL of sample for each measurement. CD spectra were obtained at room temperature by continuous wavelength scans ranging from 200 to 270 nm at a scanning speed of 50 nm per minute. Averages of three scans per sample were calculated. The spectra were typical for a triple helical conformation which is in line with earlier work established by F. Ruggiero (2000) [29](data not shown). The thermal transition curve for both SPIDICOL1 and FIB3COL1 proteins measured by circular dichroism at 225 nm indicated a Tₘ value of 41.6° C. and 43.4° C., respectively at which 50% of the SPIDICOL1 and FIB3COL1 molecules remain in a fully folded conformation as compared to 40° C. for bovine heterotrimeric Type I Collagen shown in prior art (FIG. 10). The gradual decrease in the quantity of the detected SPIDICOL1 and FIB3COL1 is due to the fact that the extent of hydroxylation can vary from one SPIDICOL1 or FIB3COL1 molecule to the other, resulting in a population of triple helices with different melting point temperatures. These results show that co-expression of the chimeric P4H and LH3 enzymes with the both the SPIDICOL1 an FIB3COL1 proteins proved to be essential for conformation and stability.

### Structural Analysis

To visualize the fibril lattice network of SPIDICOL1 and FIB3COL1, the resulting fusion proteins were allowed to assemble to fibrils, collected, and analyzed by scanning electron microscopy (SEM) (See FIG. 11). For the preparation of samples for SEM, fibril formation of the SPIDICOL1 and FIB3COL1 proteins was induced by mixing with 5 μL of fibrillogenesis buffer (60 mM NaH2PO4, 1.4% NaCl (w/v), pH 9.5) and incubating for 1 h at 37° C. The SPIDICOL1 and FIB3COL1 samples were then immersed in 0.1 M phosphate buffer (pH7.3) and 2.5% glutaraldehyde (4° C.), followed by rinsing the samples three times in phosphate buffer and gradually dehydrating them by adding increasing concentrations of ethanol (25-100%). The samples were again rinsed for another 25 min, and finally dried in a Critical Point Dryer (Leica EM CPD300). The resulting samples were gold coated (coating thickness 25 nm) using an EM ACE600 Sputter coater (Leica) and SEM images were obtained using a Camscan MX 2600 FEGSEM using an accelerating voltage of 10 kV and magnifications of 500×. Long homogeneous fibrils and lattice structures characteristic to both Spidroin-1, Fibroin-Ill and Collagen Type-I were observed, indicating proper structures of the SPIDICOL1 and FIB3COL1 proteins.

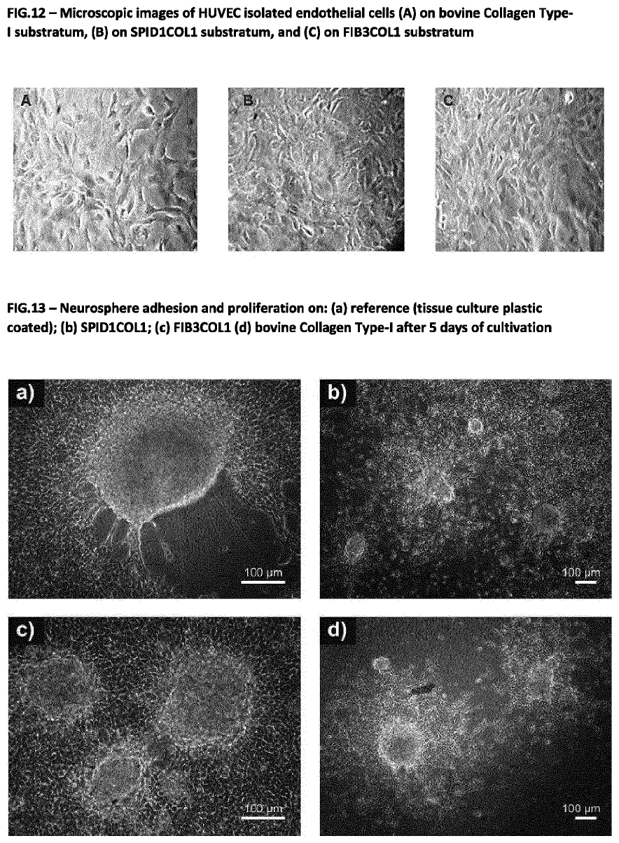

Biofunctionality In culture collagenous extracellular maxtrix proteins can bind to biological substrata and 50 simultaneously to cell surfaces, thereby promoting attachment, spreading and growth of these cells (Klebe, 1974; Pearlstein, 1976). To determine the biofunctionality of the resulting SPIDICOL1 and FIB3COL1 proteins, isolated endothelial cells derived from adult human umbilical veins (HUVEC) were seeded on antimicrobial plastic matrices precoated with either SPIDICOL1, FIB3COL1, or native human skin type I Collagen (GenoSkin). Human endothelial cells were isolated from normal, term umbilical veins as described by Gimbrone et al. (1974) [30]. Endothelial progenitor cell (EPC) yields obtained from SPIDICOL1 and FIB3COL1 were 2- and 2.5-fold higher than those obtained with native human tissue-derived collagen type I and were several fold higher than those obtained from uncoated matrices. Furthermore, the SPIDICOL1 and FIB3COL1 proteins were more effective in the isolation of cells from HUVEC isolated endothelial cell samples containing either very low or high endogenous levels of EPC. The majority of cells isolated and grown on both SPIDICOL1 and FIB3COL1 proteins appeared as typical spheroid-shaped cells supported by strong interactions with the SPIDICOL1 or FIB3COL1 protein matrix, while cells grown on either native human skin type I Collagen or uncoated matrices were mostly round. These results display that the biological activity of both SPIDICOL1 and FIB3COL1 are proven to be superior over naïve human tissue-derived collagen through its capacity to support attachment and proliferation of isolated endothelial cells derived from adult human umbilical veins. (See FIG. 12)

### Amino Acid Composition Analysis

To further verify the identity of the expressed SPIDICOL1 and FIB3COL1 proteins at amino acid composition level, samples were digested with a sulfhydryl-specific protease (ficin) and further purified which mimicked the migration of pure human skin type I collagen samples. Following electrophoretic separation of the purified SPIDICOL1 protein to Spidroin-1 and Collagen Type-1, and FIB3COL1 protein to Fibroin-Ill and Collagen Type-1, respectively, protein sequence analysis was performed on the respective bands using an LCMS-8050 triple quadrupole LC-MS/MS (Shimadzu), which were thought to correspond to both Spidroin-1 and Fibroin-Ill and Collagen Type-I (data analyzed by Traverse MS data analysis software). The bands indicated in were identified as alpha I type I collagen (Homo sapiens; p=1.08×10⁻³⁰), alpha II Type I Collagen (Homo sapiens; p=1.24×10⁻¹⁴ and Spidroin-1 (Nephila clavipes; p=1.48×10⁻¹²). All identified peptides (80% sequence coverage) displayed 100% identity to human collagen, Spidroin-1, and Fibroin-Ill protein sequences, respectively. Amino acid analysis of the resulting SPIDICOL1 and FIB3COL1 proteins showed significant identity to the human-extracted Collagen Type-I heterotrimer level [31, 32, 33] and Spidroin-1 and Fibroin-Ill level. Additionally, the hydroxylysine content was 36-fold and 39-fold higher for SPIDICOL1 and FIB3COL1, respectively, than the levels detected in LH3-free N. benthamiana plants [14] thereby establishing heterologous activity of the chimeric P4H and LH3 proteins. Measured percentages of hydroxyproline content (8.24% for SPIDICOL1 and 8.32% for FIB3COL1) were quite similar to those reported for recombinant transgenic plant-derived collagen (8.41%) performed by Merle et al. (2002) [14] and (7.55%) performed by Hanan Stein et al. (2009) [34] and hydroxylysine content (0.86%) to those of human collagen (1%), which is also in line with the 0.74% performance in the study by Hanan Stein et al. (2009) [34]. (see Table 1 for amino acid analysis of SPIDICOL1 and FIB3COL1 vs. Human-derived Collagen heterotrimers).

| | | | Human Collagen |
|---|---|---|---|
| Amino Acid | SPID1COL (%) | FIB3COL1 (%) | Type-I (%) |
| Asp + Asn | 4.11 | 4.19 | 4.3 |
| Hydroxyproline | 8.24 | 8.32 | 10.3 |
| Threonine | 1.43 | 1.47 | 1.7 |
| Serine | 3.81 | 3.63 | 3.3 |
| Glu + Gln | 7.47 | 7.51 | 7.1 |
| Proline | 15.71 | 15.94 | 12.0 |
| Glycine | 35.64 | 34.42 | 33.5 |
| Alanine | 14.82 | 14.21 | 11.1 |
| Valine | 2.71 | 2.57 | 2.6 |
| Isoleucine | 1.24 | 1.08 | 0.9 |
| Leucine | 3.37 | 2.87 | 2.3 |
| Tyrosine | 0.78 | 0.69 | 0.2 |
| Phenylalanine | 0.96 | 1.14 | 1.2 |
| Hydroxylysine | 0.89 | 0.86 | 1.0 |
| Lysine | 2.44 | 2.56 | 2.3 |
| Histidine | 0.51 | 0.49 | 0.6 |
| Arginine | 5.61 | 5.42 | 5.0 |
| Cysteine | ND | ND | ND |
| Methionine | 0.41 | 0.38 | 0.6 |
| Tryptophan | ND | ND | ND |

### Example Three: SPIDICOL1 and FIB3COL1 Electrospun Scaffolds

One of the main objectives in tissue engineering is the fabrication of cyto-compatible scaffolds and the selection of (bio)materials that can perform cell interactions to ensure the physiological activity of the construction. There is a spectrum of requirements for these materials, such as non-toxicity, low immunogenicity, a well-defined biodegradation rate, and the like. The structure of the scaffold should imitate the native extracellular matrix structure as closely as possible and perform its functions to recreate the native conditions for cells. The inventor of the present invention investigated three different scaffold constructions fabricated either with SPIDICOL1, FIB3COL1, or native Human Type I Collagen proteins. Both spider-based Spidroins and fibroins are characterized by their unique combination of physico-chemical and biological properties, and can be used in different fields of tissue engineering, both in a solo-state and in composites (e.g., SPIDICOL1 and FIB3COL1). The main advantage of spidroin or fibroin proteins when compared with other cyto-compatible materials such as collagen, are their mechanical properties [35], which ensure the Spidroin or fibroin application as a frame-reinforcing component in various constructions [36, 37] and as a composite additive to polymers with insufficient mechanical strength [38-40] or weak mechanical properties under wet conditions such as Collagens [41].

Both Spidroin-1, derived from Nephila clavipes and Fibroin-III (an analogue of Spidroin-2 [42]), derived from Araneus diadematus are characterized by the presence of a huge number of repetitive sequences in the central part (the so-called primary repeats of 25-40 amino acid residues in size) and unique sequences of 100-300 amino acid residues at the N- and C-domains. All repeats contain poly-Ala (Alaline) blocks in 4-8 amino acid residues, which alternate with Gly (Glycine) repeat regions with the GGX motif for Spidroin-I and the GPGXX motif for Fibroin-Ill (as well as Spidroin-II) [43]. Such an alteration of the hydrophobic and hydrophilic regions of molecules ensures amphiphile properties for interaction with tissues. The presence of up to 15% of proline residues in the amino acid sequence of Fibroin-Ill, which are absent in spidroin-I [44], has a significant effect on the further formation of higher-level structures and determines the various properties of these proteins. Furthermore, both Spidroin-I and Fibroin-Ill are characterized by the ability to phase transition during dehydration. This property makes it possible to ensure the structural stability of the protein in constructions that are based on them.

The electrospinning method is one of the most promising methods for fabricating scaffolds with a defined structure. Electrospun scaffolds have a multilayer fibrous structure with a high porosity and a high surface area-to-volume ratio (SA:V). Many different types of constructions based on silk proteins have been fabricated using the electrospinning method [45, 46]. It is well known in the art that electrospun collagen nanofibers are mechanically weak in nature and readily soluble in water [47, 48, 49]. Rapid degradation is not ideal for tissue engineering application as the scaffold will disappear before the cells lay out their own ECM. Thus, collagen fibers have to be cross-linked to reduce the water solubility, to improve the resistant to enzymatic degradation and to enhance the mechanical strength.

The present invention makes it possible to create cyto-compatible scaffolds using either SPIDICOL1 or FIB3COL1 proteins that both combine mechanical properties and high cytocompatibility with modification potential versus conventional animal-derived Collagen Type 1. These properties allow the requirements of tissue engineering to be satisfied. Furthermore, both SPIDICOL1 and FIB3COL1 are characterized by high strength and an elasticity modulus compared to conventional animal-derived Collagen Type I, which are necessary to accelerate regenerative potential and to reduce surgical trauma. Thus, in the course of this study, a comparative analysis of the structure, biological properties and regenerative potential of SPIDICOL1 and FIB3COL1 electrospun scaffolds vs. commercial animal-derived Collagen Type I was performed and novel data on their structure and biological properties was obtained, highlighting the obvious performance superiority of SPIDICOL1 and FIB3COL1.

### Fabrication of SPIDICOL1- and FlB3COL1-Based Scaffolds

Aqueous solutions (30% concentrations) of SPIDICOL1, FIB3COL1, and Bovine Collagen Type I proteins were dried in Petri dishes in a Critical Point Dryer (Leica EM CPD300). The dried proteins were dissolved in a phosphate buffered saline (PBS)/1,1,1,3,3,3 hexafluoro-2-propanol (HFIP)/acetic acid ternary mixture as solvent at a ratio of 1:1:1 and a rate of 50 mg/mL. HFIP, a volatile solvent (boiling point of 61° C.), evaporates under normal atmospheric conditions generating polymer fibers in a dry state [50]. This approach has been used successfully to develop various scaffolds that were assessed in both in vitro and in vivo studies [51-55]. The resulting solutions were centrifuged for 12 min at 11,500×g and then each protein was mixed separately in a volume ratio of 7:3, respectively, to a total protein concentration of 50 mg/mL. Microfibrous scaffolds were fabricated using the electrospinning method using an E-Fiber EF100 electrospinning device (SKE Research Equipment). The solutions that were loaded into CadenceScience Tuberculin glass syringens (Fisher Scientific) were deposited to the fixed collector surface (steel plate) under an electric field with a voltage of 6.7-7 kV through a standard 18 G blunt tip needle. The solution feed rate was 0.125 mL/h, and the needle-collector distance was 10 cm. The scaffolds were dried in a Critical Point Dryer (Leica EM CPD300) and were then separated. To create scaffolds for cell adhesion and proliferation research, the solutions were deposited with similar parameters on cover glasses that were attached to the collector.

### Morphology and Characterization of Electrospun Nanofibers

The structure of the SPIDICOL1, FIB3COL1, and Bovine Collagen Type I (Thermo Fisher) scaffolds 50 were analyzed using Scanning Electron Microscopy (SEM). The SEM method enabled to confirm the porous fibrous structure of the resulting scaffolds, as well as to estimate the average thickness of their fiber composition. In brief, Nanofibers were fixed in a mixture of 1.5% glutaraldehyde/3% paraformaldehyde in 100 mM sodium cacodylate buffer (pH 7.4) with 2.5% sucrose for 40 minutes at room temperature, followed by a 1% osmium tetroxide in 100 mM sodium cacodylate buffer (pH 7.4) fixation for 20 minutes at room temperature. The respective samples were dehydrated with a graded ethanol series (50/75/85/95/100% in water) followed by critical drying using a a Critical Point Dryer (Leica EM CPD300). Subsequently, the resulting samples were gold coated (coating thickness 10 nm) using an EM ACE600 Sputter coater (Leica) and SEM images were obtained using a Camscan MX 2600 FEGSEM. The average diameter of the electrospun fibers was analyzed from at least five different sections of the SEM images using Image J software, which were 630±81 nm for SPIDICOL1, 564±22 nm for FIB3COL1, and 319±29 nm for Bovine Collagen Type I in mean diameter, respectively (See FIG. 11). It was confirmed that both fiber diameter and alignment of SPIDICOL1, FIB3COL1 and Bovine Collagen Type I influenced NSC adhesion, proliferation, and differentiation thereby obviating the superiority of both SPIDICOL1 and FIB3COL1 compared to native Bovine Collagen Type I (Table 2). These tests were performed on primary Mouse Neural Stem Cells. BALB/cA mouse embryos at embryonic day 13.5-14.5 (E13.5-E14.5) were isolated after sacrifice of gravid females and placed into ice-cold Hank's balanced salt solution (HBSS, Thermo Fisher) for extraction of neural stem cells. Retrieval of the spinal cords was collected from 1 to 2 litters of embryos at a time, and rinsed in HBSS. Following rinsing, the tissue was placed in NeuroCult-XF proliferation medium (StemCell Technologies) and mechanically dissociated by repeated gentle trituration through wide bore tips (Thermo Fisher). The suspension was placed in a T75 TC treated falcon cell culture flask (Fudau) containing NeuroCult-XF proliferation medium (StemCell Technologies) and associated NeuroCult™ Proliferation Supplement (STEMCELL Technologies), as well as penicillin (100 U)/streptomycin (125 μg/mL; Thermo Fisher). Cells were grown as free-floating clustered neurospheres at 37° C. with 92% air and 8% CO₂, passaged by mechanical dissociation every 5 days, preventing from attachment (gently knocking flasks) every other day. Proliferation kinetics of the cultures were studied by microscopic examination (e.g., collecting neurospheres every 2 days, and assessing the total number of viable cells at each passage by Trypan Blue exclusion). For the microscopic examination, dark, dense spheres were considered to be unhealthy and composed of more dead cells than lighter colored spheres, as viable neurospheres are generally semitransparent. Initially, single cells proliferated to form small clusters of cells that lightly adhered to the SPIDICOL1, FIB3COL1, or Bovine Collagen Type I scaffolds; however some of these clusters lifted off as the density of the sphere increases. Cells used for transplantation or in vitro differentiation had been passaged 5 times.

TABLE 2
The influence of fiber diameter and alignment on adhesion, proliferation,
and differentiation. Cell counts were performed three days after seeding
cells on the scaffolds.

| | | Adhesion | Proliferation | Differentiation |
|---|---|---|---|---|
| Scaffold | Fiber diameter | (%) | (%) | (% Neurons) |
| SPID1COL1 | 630 ± 81 nm | 91 | 90 | 80 |
| FIB3COL1 | 564 ± 22 nm | 87 | 92 | 82 |
| Native Bovine | 319 ± 29 nm | 68 | 78 | 38 |
| Collagen Type I | | | | |

### In Vitro Differentiation and Immunocytochemisty

In vivo extracellular matrices, such as collagen and laminin, exhibit micro- to nano-scale fibrous topography, which explains why electrospun matrices significantly influence the adhesion, survival, proliferation, and differentiation of stem cells. In order to gain insight on how either SPIDICOL1, FIB3COL1, or Bovine Collagen Type I influence neural development, the aforementioned neurospheres were were seeded on either electrospun SPIDICOL1, FIB3COL1 or native Bovine Collagen Type I scaffolds to study their effect on adhesion and proliferation. Therefore, the aforementioned neurospheres were plated as small spheres onto poly-D-lysine (PDL, Sigma Aldrich), laminin coated coverslips (Thermo Fisher), or electrospun meshes three days after the last passage, in NeuroCult NS-A Differentiation medium (StemCell Technologies), as well as penicillin (100 U)/streptomycin—125 μg/mL; Thermo Fisher). The cells were differentiated for seven days and then fixed for 15 minutes in 4% paraformaldehyde (PFA) at room temperature. Following rinses in Phosphate buffered saline (PBS, pH 7.2) and block in a blocking solution of 5% normal goat serum and 0.25% Triton X-100 in 0.02 M PBS (PBS+), the cultures underwent immunocytochemistry with reaction to primary antibodies overnight at 5° C. After 5 rinses in PBS+, the cultures were further incubated in the absence of light (dark room) with Alexa Fluor 488- and 594-conjugated secondary antibodies at a 1:100 ratio (Invitrogen) in PBS+ for 2.5 hours at room temperature. After 5 rinses in PBS, 4′,6-diamidino-2-phenylindole (DAPI, Thermo Fisher) was added for 5 minutes before gently rinsing in PBS and placing the coverslip on a microscope slide. Negative controls with omission of primary antibodies were performed in parallel, and no positive signals were detected. Cells were also evaluated after 1, 5, 10, 21, and 28 days. Cell viability, estimated by trypan blue exclusion, was around 90% and 92% for the SPIDICOL1, and FIB3COL1 scaffolds at Day 3, while it was only approximately 78% for the native Bovine Collagen Type I scaffold. The small clusters observed on the native Bovine Collagen Type I scaffold were dark and dense, indicating unhealthy or dead cells. By Day 5, the neurospheres on the SPIDICOL1 and FIB3COL1 scaffolds were still mainly semi-transparent and cell viability was around 86% and 88%, respectively. Some spheres adhered to the scaffold, as the single cells were proliferating and forming small clusters of cells. The neurospheres on the native Bovine Collagen Type I scaffold did not readily adhere at the ratio SPIDICOL1 or FIB3COL1 scaffolds; as the dark, dense spheres of unhealthy or dead cells lifted off and the density of the sphere increased. Cell viability on the native Bovine Collagen Type I scaffold was only around 63% by Day 5 (FIG. 13). As both the Spidroin-I or Fibroin-Ill moiety of the SPIDICOL1 or FIB3COL1 proteins promoted a more significant profileration rate compared to native Bovine Collagen Type I, the inventor also investigated whether it also had an effect on differentiation. To test this, the aforementioned neurospheres were plated as small spheres onto either poly-D-lysine (PDL), laminin coated coverslips, or electrospun meshes three days after the sixth passage, in NeuroCult NS-A Differentiation medium (StemCell Technologies). After seven days, the neurospheres readily adhered, flattened, and spread to yield large numbers of migrating cells.

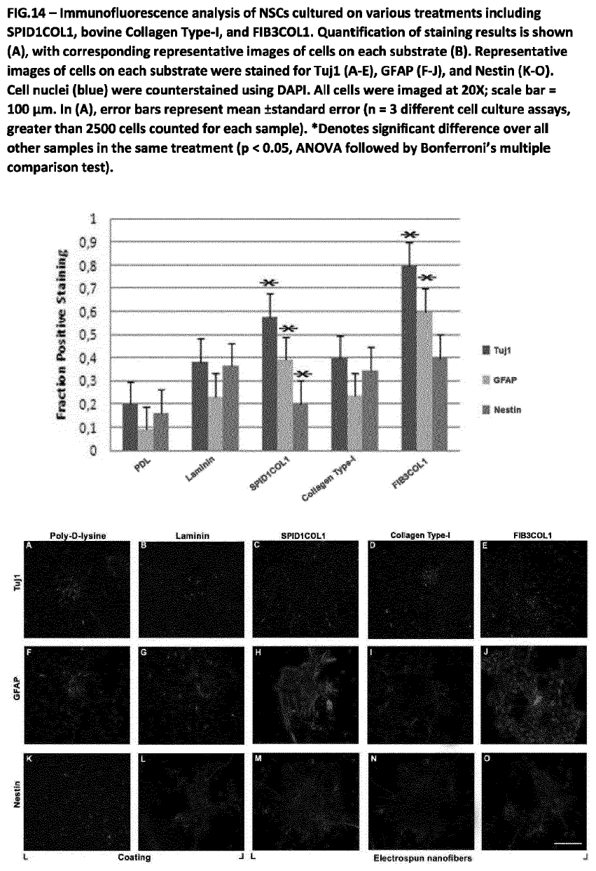

Cells were stained for neuron specific beta-Tubulin (Tuj1) to demonstrate neurons, glial fibrillary acidic protein (GFAP) for astrocytes, 04 for oligodendrocytes, and Nestin to show intermediate filament proteins to identify neuroepithelial stem cells (FIG. 14). Neurons, astrocytes, and nestin-expressing cells were observed for all treatments, but oligodendrocytes were not detected. The electrospun SPIDICOL1 and FIB3COL1 treatments displayed the highest proportion of neurons (80% for SPIDICOL1 and 82% for FIB3COL1), astrocytes (61% for SPIDICOL1 and 62% for FIB3COL1), and Nestin positive (40% for SPIDICOL1 and 41% for FIB3COL1) cells. In contrast, treatment with native Bovine Collagen Type 1 displayed much lower proportion of neurons (38%), astrocytes (14%), and Nestin positive (35%) cells. As both the electrospun SPIDICOL1 and FIB3COL1 scaffolds generated the highest proportion of neurons, astrocytes, and Nestin positive cells compared to native Bovine Collagen Type I, it has the potential to be an excellent scaffold for neural tissue engineering. In this cell culture model, the biocomposite scaffolds SPIDICOL1 and FIB3COL1 increased the proportion of cells that differentiated into neurons, astrocytes, and Nestin positive cells more substantially compared to laminin, and native Bovine Collagen Type I. The behavior of the SPIDICOL1 and FIB3COL1 scaffolds superiorly mimicked the native ECM compared to the native Bovine Collagen Type I scaffolds, and are therefore encouraging for use as a component of a therapeutic strategy to repair the injured spinal cord. (FIG. 14)

### Tensile Strength

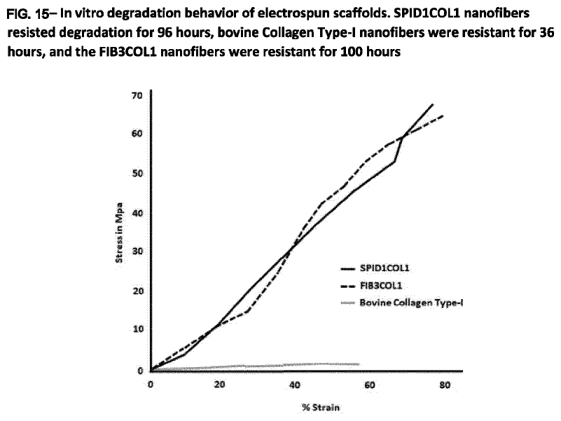

Sufficient tensile strength is essential for a peripheral nerve substitute, as it must withstand manipulation during surgery. In addition, subsequent tissue movements associated with the cardiorespiratory cycle and patient movement must be tolerated, especially when tissue begins to infiltrate the scaffolds and axonal growth increases [56]. Tensile properties of the electrospun SPIDICOL1, FIB3COL1, and Bovine Collagen Type I nanofiber scaffolds were determined using a tabletop Sauter SD 500N100 tensile tester (Imlab) at a load cell capacity of 10 N. Dogbone shaped test specimens consisting of dimensions 10 mm breadth×15 mm length, with a thickness of 500 μm were tested at a crosshead speed of 10 mm/min and gauge length of 20 mm, at room temperature [57, 58]. A minimum of 20 specimens of individual scaffolds were tested until a break was endured; the results obtained were then plotted for the determination of the stress-strain curve of the scaffolds. FIG. 15 shows the maximum stress-strain comparison for the electrospun SPIDICOL1, FIB3COL1, and native Collagen Type I nanofibers. The maximum tensile strength of the SPIDICOL1 and FIB3COL1 scaffolds were approximately 45-fold higher than the native Bovine Collagen Type I scaffold, performing at 122.51 MPa, with an average of 89.77±2.18 MPa and an ultimate strain of 84% for SPIDICOL1, and at 126.23 MPa, with an average of 91.49±3.04 MPa and an ultimate strain of 82% for FIB3COL1, compared to a 1.32 MPa, with an average of 0.54±0.68 MPa, and an elongation at break of 58% for the native Bovine Collagen Type I scaffold. The native Bovine Collagen Type I scaffolds therefore, had insufficient tensile strength to be used as a nerve graft alone, considering the well-known fact that the tensile strength of a fresh human sciatic nerve is 11.63±1.80 MPa [59]. Both the SPIDICOL1 and FIB3COL1 scaffolds show substantially improved tensile properties making them suitable for neural tissue engineering.

### Degradation

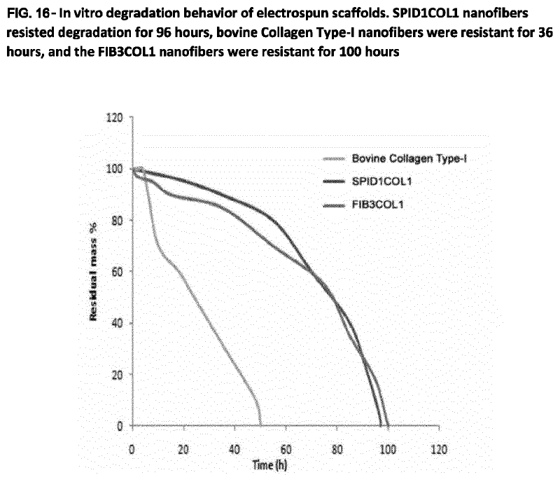

To determine the degradation rate of the SPIDICOL1, FIB3COL1 scaffolds, a combination of lipase (7 mg/mL) and collagenase (1 mg/mL) was dissolved in PBS (pH 7.4). For the native Collagen Type I scaffolds a single concentration of 1 mg/mL collagenase was used. The samples were weighed prior placement in a tube of the respective enzymatic solutions kept at 37° C. The samples were removed, blot-dried with paper cloth until the mass remained constant, and weighed after 2, 4, 8, 24 hours, and then every 24 hours, until the mass of the samples remained 50 constant. The net weight of the scaffolds were calculated by subtracting the wet chamber weights from the scaffold-containing wet chamber weights Once the initial wet well weight was reached, a value of 0 was assigned. As shown in FIG. 16, the Bovine Collagen Type I scaffolds degraded faster than both the SPIDICOL1 and FIB3COL1 scaffolds. When incubated in collagenase solution at 37° C. for 64 hours, the native Bovine Collagen Type I nanofibers were resistant for up to 36 hours and were complete degraded at 64 hours. However, both the SPIDICOL1 and FIB3COL1 scaffolds were substantially more stable and resisted both lipase and collagenase degradation as it took 100 hours for the complete degradation of the nanofibers.

Both SPIDICOL1 and FIB3COL1 scaffolds showed resistance up to 96 and 100 hours, respectively in the lipase/collagenase solution, showing their superiority towards degradation compared to native Bovine Collagen Type I.

### CONCLUSIONS

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. In addition, citation or identification of any reference in this application shall not be construed as an admission that such reference is available as prior art to the present invention.

This work provides evidence that a combination of biochemical and topographical cues can influence the direction of cellular differentiation, and raises important questions regarding fate-specification mechanisms enhanced by substrate topography. Electrospun nanofibrous scaffolds provide mechanical stability, structural guidance, and a matrix for cell integration with surrounding tissue. Collagen physically supports cells by providing specific ligands for cell adhesion, thereby acting as an ECM-mimicking nano-scaffold. The SPIDICOL1 and FIB3COL1 proteins show improved cell differentiation in vitro compared to native Collagen Type I and similar to how the native ECM does in vivo. We found increased fiber diameters, along with improved mechanical properties for both SPIDICOL1 and FIB3COL1 nanofibers compared to native Collagen Type I nanofibers, thereby allowing a proportion of desired cell types to be controlled for possible therapeutic purposes.

### REFERENCES

1. Frantz C, Stewart K M, Weaver V M. The extracellular matrix at a glance. J Cell Sci. 2010; 123(Pt 24):4195-4200. doi:10.1242/jcs.023820
2. Järveläinen H, Sainio A, Koulu M, Wight T N, Penttinen R. Extracellular matrix molecules: potential targets in pharmacotherapy. Pharmacol Rev. 2009 June; 61(2):198-223. doi: 10.1124/pr.109.001289. PMID: 19549927; PMCID: PMC2830117.
3. Schaefer L, Schaefer R M. Proteoglycans: from structural compounds to signaling molecules. Cell Tissue Res. 2010 January; 339(1):237-46. doi: 10.1007/s00441-009-0821-y. Epub 2009 Jun. 10. PMID: 19513755.
4. Alberts B., Johnson A., Lewis J., Raff M., Roberts K., Walter P. (2007). Molecular Biology of the Cell. London: Garland Science;
5. Harvey S J, Miner J H. Revisiting the glomerular charge barrier in the molecular era. Curr Opin Nephrol Hypertens. 2008 July; 17(4):393-8. doi: 10.1097/MNH.0b013e32830464de. PMID: 18660676.
6. Morita H, Yoshimura A, Inui K, Ideura T, Watanabe H, Wang L, Soininen R, Tryggvason K. Heparan sulfate of perlecan is involved in glomerular filtration. J Am Soc Nephrol. 2005 June; 16(6):1703-10. doi: 10.1681/ASN.2004050387. Epub 2005 May 4. PMID: 15872080.
7. Rozario T, DeSimone D W. The extracellular matrix in development and morphogenesis: a dynamic view. Dev Biol. 2010 May 1; 341(1):126-40. doi: 10.1016/j.ydbio.2009.10.026. Epub 2009 Oct. 23. PMID: 19854168; PMCID: PMC2854274.
8. De Wever 0, Demetter P, Mareel M, Bracke M. Stromal myofibroblasts are drivers of invasive cancer growth. Int J Cancer. 2008 Nov. 15; 123(10):2229-38. doi: 10.1002/ijc.23925. PMID: 18777559.
9. Wise S G, Weiss A S. Tropoelastin. Int J Biochem Cell Biol. 2009 March; 41(3):494-7. doi: 10.1016/j.biocel.2008.03.017. Epub 2008 Apr. 1. PMID: 18468477.
10. Lucero H A, Kagan H M. Lysyl oxidase: an oxidative enzyme and effector of cell function. Cell Mol Life Sci. 2006 October; 63(19-20):2304-16. doi: 10.1007/s00018-006-6149-9. PMID: 16909208.
11. Smith M L, Gourdon D, Little W C, Kubow K E, Eguiluz R A, Luna-Morris S, Vogel V. Force-induced unfolding of fibronectin in the extracellular matrix of living cells. PLoS Biol. 2007 Oct. 2; 5(10):e268. doi: 10.1371/journal.pbio.0050268. PMID: 17914904; PMCID: PMC1994993.
12. Trebaul A, Chan E K, Midwood K S. Regulation of fibroblast migration by tenascin-C. Biochem Soc Trans. 2007 August; 35(Pt 4):695-7. doi: 10.1042/BST0350695. PMID: 17635125.
13. Tucker R P, Chiquet-Ehrismann R. The regulation of tenascin expression by tissue microenvironments. Biochim Biophys Acta. 2009 May; 1793(5):888-92. doi: 10.1016/j.bbamcr.2008.12.012. Epub 2008 Dec. 31. PMID: 19162090.
14. Merle C, Perret S, Lacour T, Jonval V, Hudaverdian S, Garrone R, Ruggiero F, Theisen M. Hydroxylated human homotrimeric collagen I in Agrobacterium tumefaciens-mediated transient expression and in transgenic tobacco plant. FEBS Lett. 2002 Mar. 27; 515(1-3):114-8. doi: 10.1016/s0014-5793(02)02452-3.
15. Torre-Blanco A, Alvizouri A M. In vitro hydroxylation of proline in the collagen of the cysticercus of Taenia solium. Comp Biochem Physiol B. 1987; 88(4):1213-7. doi: 10.1016/0305-0491(87)90026-5.
16. Myllyharju J. Prolyl 4-hydroxylases, the key enzymes of collagen biosynthesis. Matrix Biol. 2003 March; 22(1):15-24. doi: 10.1016/s0945-053x(03)00006-4.
17. Berg R A, Prockop D J. The thermal transition of a non-hydroxylated form of collagen. Evidence for a role for hydroxyproline in stabilizing the triple-helix of collagen. Biochem Biophys Res Commun. 1973 May 1; 52(1):115-20. doi: 10.1016/0006-291x(73)90961-3.
18. Annunen P, Helaakoski T, Myllyharju J, Veijola J, Pihlajaniemi T and Kivirikko K I (1997) Cloning of the human prolyl 4-hydroxylase a subunit isoform a(I I) and characterization of the type I I enzyme tetramer. The a(1) and a(I I) subunits do not form a mixed a(I) a(I I) P2 tetramer. J Biol Chem, 272, 17342-17348.
19. Gorres K L, Raines R T. Prolyl 4-hydroxylase. Crit Rev Biochem Mol Biol. 2010 April; 45(2):106-24. doi: 10.3109/10409231003627991.
20. Myllyharju, J. & Kivirikko, K. I. Collagens, modifying enzymes and their mutations in humans, flies and worms. Trends Genet. 20, 33-43 (2004).
21. Guengerich F P. Introduction: Metals in Biology: α-Ketoglutarate/Iron-Dependent Dioxygenases. J Biol Chem. 2015; 290(34):20700-20701. doi:10.1074/jbc.R115.675652
22. Ibrahimi A, Vande Velde G, Reumers V, Toelen J, Thiry I, Vandeputte C, Vets S, Deroose C, Bormans G, Baekelandt V, Debyser Z, Gijsbers R. Highly efficient multicistronic lentiviral vectors with peptide 2A sequences. Hum Gene Ther. 2009 August; 20(8):845-60. doi: 10.1089/hum.2008.188.
23. Weber, E., Engler, C., Gruetzner, R., Werner, S., and Marillonnet, S. (2011). A modular cloning system for standardized assembly of multigene constructs. PLoS One 6:e16765. doi: 10.1371/journal.pone.0016765
24. Stavolone L, Kononova M, Pauli S, Ragozzino A, de Haan P, Milligan S, Lawton K, Hohn T. Cestrum yellow leaf curling virus (CmYLCV) promoter: a new strong constitutive promoter for heterologous gene expression in a wide variety of crops. Plant Mol Biol. 2003 November; 53(5):663-73. doi: 10.1023/B:PLAN.0000019110.95420.bb.
25. Nagaya S, Kawamura K, Shinmyo A, Kato K. The HSP terminator of Arabidopsis thaliana increases gene expression in plant cells. Plant Cell Physiol. 2010 February; 51(2):328-32. doi: 10.1093/pcp/pcp188.
26. Liu Z, Chen O, Wall JBJ, Zheng M, Zhou Y, Wang L, Vaseghi H R, Qian L, Liu J. Systematic comparison of 2A peptides for cloning multi-genes in a polycistronic vector. Sci Rep. 2017 May 19; 7(1):2193. doi: 10.1038/s41598-017-02460-2.
27. Baltes N J, Gil-Humanes J, Cermak T, Atkins P A, Voytas D F. DNA replicons for plant genome engineering. Plant Cell. 2014 January; 26(1):151-63. doi: 10.1105/tpc.113.119792. Epub 2014 Jan. 17. PMID: 24443519; PMCID: PMC3963565.
28. P. Bruckner, D. J. Prockop Anal. Biochem., 110 (1981), pp. 360-368
29. F. Ruggiero, J.-Y. Exposito, P. Bournat, V. Gruber, S. Perret, J. Comte, B. Olagnier, R. Garrone, M. Theisen FEBS Lett., 469 (2000), pp. 132-136
30. Michael A. Gimbrone, Jr.,Ramzi S. Cotran, Judah Folkman J Cell Biol (1974) 60 (3): 673-684. https://doi.org/10.1083/jcb.60.3.673
31. Nokelainen M, Tu H, Vuorela A, Notbohm H, Kivirikko K I, Myllyharju J. High-level production of human type I collagen in the yeast Pichia pastoris. Yeast. 2001 Jun. 30; 18(9):797-806. doi: 10.1002/yea.730.
32. Myllyharju J, Nokelainen M, Vuorela A, Kivirikko K I. Expression of recombinant human type 1-111 collagens in the yeast Pichia pastoris. Biochem Soc Trans. 2000; 28(4):353-7. PMID: 10961918.
33. Vuorela A, Myllyharju J, Nissi R, Pihlajaniemi T, Kivirikko K I. Assembly of human prolyl 4-hydroxylase and type 11 collagen in the yeast Pichia pastoris: formation of a stable enzyme tetramer requires coexpression with collagen and assembly of a stable collagen requires coexpression with prolyl 4-hydroxylase. EMBO J. 1997 Nov. 17; 16(22):6702-12. doi: 10.1093/emboj/16.22.6702.
34. Stein H, Wilensky M, Tsafrir Y, Rosenthal M, Amir R, Avraham T, Ofir K, Dgany O, Yayon A, Shoseyov O. Production of bioactive, post-translationally modified, heterotrimeric, human recombinant type-I collagen in transgenic tobacco. Biomacromolecules. 2009 Sep. 14; 10(9):2640-5. doi: 10.1021/bm900571b.
35. Stoppato, M.; Stevens, H. Y.; Carletti, E.; Migliaresi, C.; Motta, A.; Guldberg, R. E. Effects of silk fibroin fiber incorporation on mechanical properties, endothelial cell colonization and vascularization of PDLLA scaffolds. Biomaterials 2013, 34, 4573-4581
36. Mobini, S.; Hoyer, B.; Solati-Hashjin, M.; Lode, A.; Nosoudi, N.; Samadikuchaksaraei, A.; Gelinsky, M. Fabrication and characterization of regenerated silk scaffolds reinforced with natural silk fibers. J. Biomed. Mater. Res. A 2013, 101, 2392-2404.
37. Park, S.; Edwards, S.; Hou, S.; Boudreau, R.; Yee, R.; Jeong, K. J. Multi-interpenetrating network (ipn) hydrogel by gelatin and silk fibroin. Biomater. Sci. 2019, 7, 1276-1280.
38. Panas-Perez, E.; Gatt, C. J.; Dunn, M. G. Development of a silk and collagen fiber scaffold for anterior cruciate ligament reconstruction. J. Mater. Sci. Mater. Med. 2013, 24, 257-265
39. Ghezzi, C. E.; Marelli, B.; Muja, N.; Hirota, N.; Martin, J. G.; Barralet, J. E.; Alessandrino, A.; Freddi, G.; Nazhat, S. N. Mesenchymal stem cell-seeded multilayered dense collagen-silk fibroin hybrid for tissue engineering applications. Biotechnol. J. 2011, 6, 1198-1207
40. Vasconcelos, A.; Gomes, A. C.; Cavaco-Paulo, A. Novel silk fibroin/elastin wound dressing. Acta Biomater. 2012, 8, 3049-3060
41. Shunji Yunoki, Toshiyuki Ikoma, Junzo Tanaka. Development of collagen condensation method to improve mechanical strength of tissue engineering scaffolds,Material Characterization, Volume 61, Issue 9, 2010, Pages 907-911, ISSN 1044-5803, https://doi.org/10.1016/j.matchar.2010.05.010.
42. Gatesy, J.; Hayashi, C.; Motriuk, D.; Woods, J.; Lewis, R. Extreme diversity, conservation, and convergence of spider silk fibroin sequences. Science 2001, 291, 2603-2605
43. R6mer L, Scheibel T. The elaborate structure of spider silk: structure and function of a natural high performance fiber. Prion. 2008; 2(4):154-161. doi:10.4161/pri.2.4.7490
44. Hayashi, C. Y.; Shipley, N. H.; Lewis, R. V. Hypotheses that correlate the sequence, structure, and mechanical properties of spider silk proteins. Int. J. Biol. Macromol. 1999,24,271-275
45. Zhao, L.; Chen, D.; Yao, Q.; Li, M. Studies on the use of recombinant spider silk protein/polyvinyl alcohol electrospinning membrane as wound dressing. Int. J. Nanomed. 2017, 12, 8103-8114
46. Meng, Z. X.; Wang, Y. S.; Ma, C.; Zheng, W.; Li, L.; Zheng, Y. F. Electrospinning of PLGA/gelatin randomly oriented and aligned nanofibers as potential scaffold in tissue engineering. Mater. Sci. Eng. 2010, 30, 1204-1210
47. Telemeco T, Ayres C, Bowlin G, Wnek G, Boland E, Cohen N, et al. Regulation of cellular infiltration into tissue engineering scaffolds composed of submicron diameter fibrils produced by electrospinning. Acta Biomater. 2005; 1:377-385. doi: 10.1016/j.actbio.2005.04.006
48. Rho K S, Jeong L, Lee G, Seo B M, Park Y J, Hong S D, et al. Electrospinning of collagen nanofibers: effects on the behavior of normal human keratinocytes and early-stage wound healing. Biomaterials. 2006; 27:1452-1461. doi: 10.1016/j.biomaterials.2005.08.004.
49. Buttafoco L, Kolkman N, Engbers-Buijtenhuijs P, Poot A, Dijkstra P, Vermes I, et al. Electrospinning of collagen and elastin for tissue engineering applications. Biomaterials. 2006; 27:724-734. doi: 10.1016/j.biomaterials.2005.06.024.
50. Matthews J A, Wnek G E, Simpson D G, Bowlin G L. Electrospinning of collagen nanofibers. Biomacromolecules 2002; 3:232-238.
51. Zhang X, Reagan M R, Kaplan D L. Electrospun silk biomaterial scaffolds for regenerative medicine. Adv Drug Deliv Rev 2009; 61:988-1006.
52. Boland E D, Matthews J A, Pawlowski K J, Simpson D G, Wnek G E, Bowlin G L. Electrospinning collagen and elastin: preliminary vascular tissue engineering. Front Biosci 2004; 9:1422-1432.
53. Rho K S, Jeong L, Lee G, Seo B, Park Y J, Hong S, Roh S, Cho J J, Park W H, Min B. Electrospinning of collagen nanofibers: Effects on the behavior of normal human keratinocytes and early-stage wound healing. Biomaterials 2006; 27:1452-1461.
54. Zhang X, Baughman C B, Kaplan D L. In vitro evaluation of electrospun silk fibroin scaffolds for vascular cell growth. Biomaterials 2008; 29:2217-2227.
55. Noh H K, Lee S W, Kim J, Oh J, Kim K, Chung C, Choi S, Park W H, Min B. Electrospinning of chitin nanofibers: Degradation behavior and cellular response to normal human keratinocytes and fibroblasts. Biomaterials 2006; 27:3934-3944.
56. Ma, M.; Wei, P.; Wei, T.; Ransohoff, R. M.; Jakeman, L. B. Enhanced axonal growth into a spinal cord contusion injury site in a strain of mouse (129X1/SvJ) with a diminished inflammatory response. J. Comp. Neurol. 2004, 474, 469-486.
57. Prabhakaran, M. P.; Venugopal, J.; Chan, C. K.; Ramakrishna, S. Surface modified electrospun nanofibrous scaffolds for nerve tissue engineering. Nanotechnology 2008, 19, 455102-455109.
58. Mobarakeh, L. G.; Prabhakaran, M. P.; Morshed, M.; Esfahani, M. H. N.; Ramakrishna, S. Electropsun PCL/gelatin nanofibrous scaffolds for nerve tissue engineering. Biomaterials 2008, 29, 4532-4539.
59. Borschel G H, Kia K F, Kuzon W M Jr, Dennis R G. Mechanical properties of acellular peripheral nerve. J Surg Res. 2003 October; 114(2):133-9. doi: 10.1016/s0022-4804(03)00255-5.
60. Perona R. Cell signalling: growth factors and tyrosine kinase receptors. Clin Transl Oncol. 2006; 8(2):77-82. doi:10.1007/s12094-006-0162-1
61. Richardson, S. M. Tissue engineering today, not tomorrow. Regen. Med. 2007, 2, 91-94
62. Sahoo, S.; Ang, L. T.; Goh, J. C. H.; Toh, S. L. Growth factor delivery through electrospun nanofibers in scaffolds for tissue engineering applications. J. Biomed. Mater. Res. Part A 2009, 4, 1539-1550.

SEQUENCE LISTING
SEQ NO 1: Amino Acid Sequence of Collagen Type I Alpha I

| | | | | | |
|---|---|---|---|---|---|
| 10 | 20 | 30 | 40 | 50 | 60 |
| QLSYGYDEKS | TGGISVPGPM | GPSGPRGLPG | PPGAPGPQGF | QGPPGEPGEP | GASGPMGPRG |
| 70 | 80 | 90 | 100 | 110 | 120 |
| PPGPPGKNGD | DGEAGKPGRP | GERGPPGPQG | ARGLPGTAGL | PGMKGHRGFS | GLDGAKGDAG |
| 130 | 140 | 150 | 160 | 170 | 180 |
| PAGPKGEPGS | PGENGAPGQM | GPRGLPGERG | RPGAPGPAGA | RGNDGATGAA | GPPGPTGPAG |
| 190 | 200 | 210 | 220 | 230 | 240 |
| PPGFPGAVGA | KGEAGPQGPR | GSEGPQGVRG | EPGPPGPAGA | AGPAGNPGAD | GQPGAKGANG |
| 250 | 260 | 270 | 280 | 290 | 300 |
| APGIAGAPGF | PGARGPSGPQ | GPGGPPGPKG | NSGEPGAPGS | KGDTGAKGEP | GPVGVQGPPG |
| 310 | 320 | 330 | 340 | 350 | 360 |
| PAGEEGKRGA | RGEPGPTGLP | GPPGERGGPG | SRGFPGADGV | AGPKGPAGER | GSPGPAGPKG |
| 370 | 380 | 390 | 400 | 410 | 420 |
| SPGEAGRPGE | AGLPGAKGLT | GSPGSPGPDG | KTGPPGPAGQ | DGRPGPPGPP | GARGQAGVMG |
| 430 | 440 | 450 | 460 | 470 | 480 |
| FPGPKGAAGE | PGKAGERGVP | GPPGAVGPAG | KDGEAGAQGP | PGPAGPAGER | GEQGPAGSPG |
| 490 | 500 | 510 | 520 | 530 | 540 |
| FQGLPGPAGP | PGEAGKPGEQ | GVPGDLGAPG | PSGARGERGF | PGERGVQGPP | GPAGPRGANG |
| 550 | 560 | 570 | 580 | 590 | 600 |
| APGNDGAKGD | AGAPGAPGSQ | GAPGLQGMPG | ERGAAGLPGP | KGDRGDAGPK | GADGSPGKDG |
| 610 | 620 | 630 | 640 | 650 | 660 |
| VRGLTGPIGP | PGPAGAPGDK | GESGPSGPAG | PTGARGAPGD | RGEPGPPGPA | GFAGPPGADG |
| 670 | 680 | 690 | 700 | 710 | 720 |
| QPGAKGEPGD | AGAKGDAGPP | GPAGPAGPPG | PIGNVGAPGA | KGARGSAGPP | GATGFPGAAG |
| 730 | 740 | 750 | 760 | 770 | 780 |
| RVGPPGPSGN | AGPPGPPGPA | GKEGGKGPRG | ETGPAGRPGE | VGPPGPPGPA | GEKGSPGADG |
| 790 | 800 | 810 | 820 | 830 | 840 |
| PAGAPGTPGP | QGIAGQRGVV | GLPGQRGERG | FPGLPGPSGE | PGKQGPSGAS | GERGPPGPMG |
| 850 | 860 | 870 | 880 | 890 | 900 |
| PPGLAGPPGE | SGREGAPGAE | GSPGRDGSPG | AKGDRGETGP | AGPPGAPGAP | GAPGPVGPAG |
| 910 | 920 | 930 | 940 | 950 | 960 |
| KSGDRGETGP | AGPAGPVGPV | GARGPAGPQG | PRGDKGETGE | QGDRGIKGHR | GFSGLQGPPG |
| 970 | 980 | 990 | 1000 | 1010 | 1020 |
| PPGSPGEQGP | SGASGPAGPR | GPPGSAGAPG | KDGLNGLPGP | IGPPGPRGRT | GDAGPVGPPG |
| 1030 | 1040 | 1050 | | | |
| PPGPPGPPGP | PSAGFDESFL | PQPPQEKAHD | GGRYYRA | | |

SEQ NO 2: Amino Acid Sequence of Collagen Type I Alpha II

| | | | | | |
|---|---|---|---|---|---|
| 10 | 20 | 30 | 40 | 50 | 60 |
| QYDGKGVGLG | PGPMGLMGPR | GPPGAAGAPG | PQGFQGPAGE | PGEPGQTGPA | GARGPAGPPG |
| 70 | 80 | 90 | 100 | 110 | 120 |
| KAGEDGHPGK | PGRPGERGVV | GPQGARGFPG | TPGLPGFKGI | RGHNGLDGLK | GQPGAPGVKG |
| 130 | 140 | 150 | 160 | 170 | 180 |
| EPGAPGENGT | PGQTGARGLP | GERGRVGAPG | PAGARGSDGS | VGPVGPAGPI | GSAGPPGFPG |
| 190 | 200 | 210 | 220 | 230 | 240 |
| APGPKGEIGA | VGNAGPAGPA | GPRGEVGLPG | LSGPVGPPGN | PGANGLTGAK | GAAGLPGVAG |
| 250 | 260 | 270 | 280 | 290 | 300 |
| APGLPGPRGI | PGPVGAAGAT | GARGLVGEPG | PAGSKGESGN | KGEPGSAGPQ | GPPGPSGEEG |
| 310 | 320 | 330 | 340 | 350 | 360 |
| KRGPNGEAGS | AGPPGPPGLR | GSPGSRGLPG | ADGRAGVMGP | PGSRGASGPA | GVRGPNGDAG |
| 370 | 380 | 390 | 400 | 410 | 420 |
| RPGEPGLMGP | RGLPGSPGNI | GPAGKEGPVG | LPGIDGRPGP | IGPAGARGEP | GNIGFPGPKG |
| 430 | 440 | 450 | 460 | 470 | 480 |
| PTGDPGKNGD | KGHAGLAGAR | GAPGPDGNNG | AQGPPGPQGV | QGGKGEQGPP | GPPGFQGLPG |
| 490 | 500 | 510 | 520 | 530 | 540 |
| PSGPAGEVGK | PGERGLHGEF | GLPGPAGPRG | ERGPPGESGA | AGPTGPIGSR | GPSGPPGPDG |
| 550 | 560 | 570 | 580 | 590 | 600 |
| NKGEPGVVGA | VGTAGPSGPS | GLPGERGAAG | IPGGKGEKGE | PGLRGEIGNP | GRDGARGAPG |
| 610 | 620 | 630 | 640 | 650 | 660 |
| AVGAPGPAGA | TGDRGEAGAA | GPAGPAGPRG | SPGERGEVGP | AGPNGFAGPA | GAAGQPGAKG |
| 670 | 680 | 690 | 700 | 710 | 720 |
| ERGAKGPKGE | NGVVGPTGPV | GAAGPAGPNG | PPGPAGSRGD | GGPPGMTGFP | GAAGRTGPPG |
| 730 | 740 | 750 | 760 | 770 | 780 |
| PSGISGPPGP | PGPAGKEGLR | GPRGDQGPVG | RTGEVGAVGP | PGFAGEKGPS | GEAGTAGPPG |
| 790 | 800 | 810 | 820 | 830 | 840 |
| TPGPQGLLGA | PGILGLPGSR | GERGLPGVAG | AVGEPGPLGI | AGPPGARGPP | GAVGSPGVNG |
| 850 | 860 | 870 | 880 | 890 | 900 |
| APGEAGRDGN | PGNDGPPGRD | GQPGHKGERG | YPGNIGPVGA | AGAPGPHGPV | GPAGKHGNRG |
| 910 | 920 | 930 | 940 | 950 | 960 |
| ETGPSGPVGP | AGAVGPRGPS | GPQGIRGDKG | EPGEKGPRGL | PGLKGHNGLQ | GLPGIAGHHG |
| 970 | 980 | 990 | 1000 | 1010 | 1020 |
| DQGAPGSVGP | AGPRGPAGPS | GPAGKDGRTG | HPGTVGPAGI | RGPQGHQGPA | GPPGPPGPPG |
| 1030 | 1040 | | | | |
| PPGVSGGGYD | FGYDGDFYRA | | | | |

```
SEQ NO 3: Nucleotide Sequence of Collagen Type I Alpha I, Codon
Optimized for Nicotiana
Benthamiana Chloroplast Expression
      1 CAGTTGTCTT ATGGTTATGA TGAAAAATCA ACTGGAGGAA TTAGTGTTCC AGGTCCAATG
    61 GGACCATCTG GACCAAGAGG TCTTCCTGGA CCTCCAGGTG CTCCTGGTCC ACAGGGTTTT
  121 CAGGGACCAC CAGGAGAACC AGGAGAGCCA GGAGCTTCAG GTCCTATGGG TCCAAGAGGT
  181 CCACCTGGCC CTCCAGGAAA GAATGGTGAT GATGGAGAAG CAGGAAAGCC TGGTCGTCCA
  241 GGCGAAAGAG GTCCTCCTGG ACCACAAGGG GCTAGAGGAC TGCCTGGTAC TGCTGGACTT
  301 CCAGGAATGA AAGGTCATAG AGGTTTTTCT GGACTTGACG GTGCTAAGGG AGATGCAGGA
  361 CCAGCTGGAC CTAAGGGTGA GCCAGGATCT CCAGGCGAGA ACGGAGCCCC TGGTCAGATG
  421 GGACCAAGAG GATTGCCAGG TGAAAGAGGA AGGCCTGGAG CTCCTGGTCC AGCTGGAGCT
  481 AGGGGTAATG ATGGAGCTAC TGGAGCTGCA GGACCTCCTG GTCCAACTGG TCCTGCTGGA
  541 CCACCAGGTT TTCCTGGAGC TGTGGGAGCT AAAGGTGAGG CTGGTCCACA AGGTCCTAGA
  601 GGATCAGAAG GACCCCAAGG AGTTAGAGGA GAACCAGGTC CACCTGGACC AGCCGGTGCA
  661 GCTGGTCCTG CTGGTAATCC TGGTGCTGAC GGACAACCTG GCGCTAAAGG TGCAAACGGA
  721 GCTCCTGGAA TCGCAGGTGC TCCAGGTTTT CCAGGTGCAA GAGGTCCTAG TGGTCCACAG
  781 GGTCCAGGAG GTCCACCAGG ACCAAAGGGT AATAGTGGTG AGCCTGGAGC TCCAGGAAGC
  841 AAAGGAGATA CTGGTGCTAA GGGCGAACCA GGACCAGTTG GAGTGCAAGG ACCTCCAGGA
  901 CCAGCAGGAG AAGAAGGTAA GAGAGGAGCT AGGGGAGAAC CAGGACCTAC TGGTTTGCCA
  961 GGACCACCAG GTGAACGTGG AGGACCTGGA TCAAGGGGTT TTCCAGGAGC TGATGGGGTT
1021 GCTGGTCCTA AGGGACCAGC AGGAGAAAGA GGATCTCCAG GTCCTGCTGG ACCAAAAGGA
1081 AGTCCTGGAG AAGCTGGCAG ACCTGGAGAA GCAGGTCTTC CAGGTGCTAA GGGTCTTACT
1141 GGATCTCCAG GATCTCCTGG TCCTGATGGA AAAACTGGAC CACCAGGTCC TGCTGGACAA
1201 GACGGTAGAC CTGGACCTCC TGGTCCACCT GGAGCTAGAG GTCAAGCTGG TGTTATGGGA
1261 TTTCCTGGAC CAAAGGGTGC TGCTGGTGAA CCAGGGAAAG CTGGTGAAAG AGGAGTGCCT
1321 GGTCCACCTG GAGCTGTTGG CCCTGCTGGA AAGGATGGTG AAGCTGGTGC TCAAGGACCA
1381 CCAGGACCTG CTGGTCCTGC TGGAGAAAGA GGTGAGCAGG GACCAGCTGG AAGTCCTGGT
1441 TTTCAAGGAT TGCCAGGACC AGCTGGTCCT CCAGGGGAAG CAGGTAAGCC AGGCGAACAA
1501 GGAGTCCCAG GAGATTTGGG AGCTCCTGGA CCATCTGGAG CAAGAGGTGA AAGAGGATTT
1561 CCAGGAGAAA GAGGAGTTCA GGGTCCGCCA GGACCTGCTG GACCAAGAGG AGCAAACGGA
1621 GCACCAGGAA ATGATGGAGC TAAGGGGGAT GCTGGTGCTC CAGGTGCACC TGGATCTCAA
1681 GGAGCTCCAG GACTCCAAGG AATGCCTGGT GAAAGAGGTG CTGCTGGTCT TCCAGGACCT
1741 AAGGGAGATA GAGGAGATGC AGGACCAAAG GGAGCTGATG GAAGCCCTGG TAAGGATGGT
1801 GTTAGAGGAC TTACTGGACC AATAGGTCCT CCTGGTCCAG CTGGAGCACC TGGGGATAAG
1861 GGTGAGAGTG GTCCTTCTGG TCCTGCAGGC CCAACAGGAG CAAGAGGTGC TCCTGGTGAT
1921 AGAGGTGAAC CTGGACCTCC AGGTCCTGCT GGATTTGCTG GTCCACCTGG TGCTGATGGA
1981 CAACCTGGAG CAAAGGGAGA GCCTGGAGAT GCAGGAGCAA AAGGAGATGC TGGTCCACCT
2041 GGACCAGCTG GTCCTGCTGG TCCTCCTGGA CCAATCGGTA ATGTTGGAGC TCCTGGTGCT
2101 AAAGGTGCTA GGGGTTCAGC TGGACCTCCT GGAGCTACTG GTTTTCCTGG TGCTGCTGGC
2161 AGGGTTGGAC CACCTGGTCC AAGTGGAAAT GCCGGACCAC CTGGCCCACC AGGACCAGCT
2221 GGAAAAGAAG GTGGAAAAGG ACCAAGAGGA GAAACTGGTC CAGCAGGTCG TCCAGGTGAA
2281 GTGGGCCCTC CAGGCCCACC AGGACCTGCT GGAGAAAAGG GAAGTCCAGG TGCAGATGGA
2341 CCAGCTGGCG CTCCTGGTAC TCCAGGACCT CAGGGTATCG CTGGACAAAG AGGTGTTGTT
2401 GGTTTGCCAG GTCAGAGAGG AGAGAGAGGT TTTCCAGGAT TGCCAGGTCC TTCTGGTGAG
2461 CCTGGTAAAC AGGGTCCTTC TGGAGCTTCT GGTGAAAGAG GACCTCCTGG TCCTATGGGT
2521 CCACCAGGAT TGGCAGGACC ACCAGGTGAA TCTGGAAGAG AAGGTGCACC AGGAGCAGAA
2581 GGATCTCCAG GTAGGGATGG AAGCCCTGGG GCTAAAGGAG ATAGGGGAGA AACTGGACCA
2641 GCAGGACCAC CAGGTGCTCC TGGTGCCCCA GGTGCTCCTG GACCAGTTGG TCCTGCTGGT
2701 AAGTCTGGTG ACAGAGGTGA AACTGGGCCA GCTGGACCAG CTGGACCTGT TGGTCCTGTT
2761 GGTGCTAGAG GTCCAGCTGG ACCTCAAGGT CCTAGAGGAG ATAAAGGAGA AACTGGTGAA
2821 CAAGGTGATA GGGGTATTAA GGGTCATAGG GGATTTTCTG GTTTGCAAGG ACCACCTGGA
2881 CCACCAGGTT CACCAGGAGA GCAAGGTCCA AGTGGAGCAT CTGGACCAGC TGGTCCAAGG
2941 GGACCTCCTG GATCTGCTGG AGCTCCAGGT AAAGATGGAC TTAATGGTCT TCCAGGTCCA
3001 ATTGGACCTC CTGGACCAAG AGGAAGAACT GGAGATGCAG GACCAGTTGG ACCACCAGGT
3061 CCACCAGGAC CTCCTGGTCC TCCAGGACCT CCAAGTGCAG GTTTTGATTT TTCATTTCTT
3121 CCTCAACCAC CACAAGAGAA GGCTCACGAT GGAGGAAGGT ATTATAGAGC TTAA
SEQ NO 4: Nucleotide Sequence of Collagen Type I Alpha II, Codon
Optimized for Nicotiana
Benthamiana Chloroplast Expression
      1 CAATACGATG GAAAAGGAGT TGGTCTCGGA TGGGTTTGAT CCAGGTCCAA GGGACCAAGA
    61 GGTCCTCCAG GAGCTGCTGG TGCTCCTGGT CCTCAAGGAT TTCAAGGACC AGCTGGAGAG
  121 CCAGGTGAGC CTGGACAGAC TGGTCCAGCT GGTGCAAGAG GACCTGCAGG ACCTCCCGGT
  181 AAAGCTGGTG AAGATGGACA TCCAGGAAAA CCAGGAAGAC CAGGAGAGAG GGGTGTCGTT
  241 GGACCACAAG GTGCAAGAGG TTTTCCAGGA ACACCAGGTC TTCCTGGTTT TAAAGGTATT
  301 AGAGGGCACA ATGGTTTGGA TGGTTTGAAG GGTCAACCAG GTGCTCCAGG AGTTAAAGGA
  361 GAACCTGGTG CTCCAGGTGA AAATGGTACT CCGGGACAAA CTGGAGCAAG AGGATTGCCT
  421 GGAGAAAGGG GTCGTGTTGG TGCACCAGGT CCTGCAGGAG CAAGAGGTTC AGATGGATCT
  481 GTGGGTCCAG TTGGTCCTGC TGGACCAATT GGTTCTGCTG GTCCTCCTGG ATTTCCAGGA
  541 GCTCCTGGAC CAAAGGGAGA AATTGGAGCA GTTGGAAATG CAGGACCTGC TGGTCCTGCT
  601 GGACCTAGAG GTGAAGTTGG ATTGCCTGGT TTGTCGGGCC CAGTAGGTCC TCCAGGAAAT
  661 CCAGGAGCTA ATGGATTGAC TGGTGCTAAA GGAGCTGCTG GATTGCCTGG TGTGGCAGGT
  721 GCTCCTGGTC TTCCAGGTCC TAGAGGCATT CCTGGTCCAG TAGGAGCTGC AGGAGCTACT
  781 GGTGCAAGAG GTCTTGTTGG AGAACCAGGA CCCGCAGGTT CAAAAGGAGA ATCTGGAAAT
  841 AAAGGTGAAC CAGGATCTGC TGGACCTCAG GGTCCACCTG GTCCTAGTGG TGAAGAAGGA
  901 AAGAGAGGAC CTAATGGTGA GGCCGGAAGC GCTGGTCCTC CTGGACCACC AGGTCTTAGA
  961 GGAAGTCCTG GTAGTAGAGG ATTGCCAGGA GCAGATGGAA GAGCTGGTGT TATGGGACCA
1021 CCAGGTTCTA GAGGAGCTAG CGGACCAGCT GGAGTGAGGG GTCCAAATGG AGATGCTGGA
1081 AGGCCTGGAG AACCAGGATT GATGGGTCCT AGGGGTTTAC CAGGAAGTCC AGGAAATATT
1141 GGACCAGCAG GTAAAGAAGG ACCTGTGGGT TTGCCAGGAA TTGATGGAAG GCCAGGACCA
1201 ATTGGACCAG CTGGTGCTAG AGGAGAGCCT GGTAATATTG GTTTTCCAGG TCCAAAGGGT
1261 CCAACTGGAG ACCCTGGAAA GAACGGTGAT AAAGGACATG CAGGACTTGC TGGAGCAAGA
1321 GGAGCTCCTG GCCCTGATGG TAATAATGGT GCTCAAGGTC CTCCAGGACC ACAAGGTGTT
1381 CAAGGAGGAA AAGGTGAGCA AGGACCACCT GGACCACCAG GTTTTCAAGG ACTTCCTGGC
1441 CCATCTGGTC CAGCTGGTGA AGTTGGAAAA CCAGGAGAGA GAGGTCTTCA TGGAGAATTT
1501 GGACTTCCAG GACCAGCTGG CCCTAGAGGA GAAAGAGGAC CTCCAGGTGA ATCTGGTGCT
1561 GCAGGTCCAA CTGGACCAAT TGGTTCCAGA GGACCATCCG GACCTCCTGG ACCAGATGGA
1621 AATAAAGGTG AACCAGGAGT TGTGGGTGCT GTTGGTACAG CAGGTCCATC AGGTCCATCT
1681 GGTCTTCCAG GAGAGAGGGG CGCTGCTGGT ATTCCTGGTG GAAAGGGAGA GAAGGGCGAA
1741 CCAGGACTCA GAGGTGAAAT TGGAAATCCC GGAAGAGATG GAGCAAGAGG AGCTCCTGGA
1801 GCTGTTGGTG CTCCAGGACC AGCTGGTGCA ACAGGTGATA GGGGTGAAGC TGGGGCTGCT
1861 GGACCTGCTG GACCAGCTGG TCCTAGGGGT TCTCCTGGAG AAAGAGGTGA GGTAGGTCCT
1921 GCTGGACCTA ATGGTTTTGC TGGGCCAGCC GGTGCTGCTG GACAACCAGG AGCCAAGGGA
1981 GAGAGAGGAG CTAAAGGACC AAAAGGAGAG AATGGAGTCG TTGGTCCTAC TGGACCAGTT
2041 GGAGCTGCTG GACCAGCTGG ACCAAATGGA CCACCAGGAC CAGCTGGATC TAGAGGAGAT
2101 GGTGGACCAC CAGGTATGAC AGGTTTCCCA GGTGCAGCTG GAAGGACTGG ACCTCCAGGG
2161 CCATCAGGTA TTTCTGGACC TCCAGGACCA CCAGGTCCAG CTGGAAAAGA GGGTCTCAGA
2221 GGACCAAGAG GAGATCAAGG ACCAGTGGGA AGAACAGGTG AAGTGGGTGC TGTGGGTCCA
2281 CCTGGTTTTG CTGGGGAGAA AGGTCCTTCC GGAGAAGCTG GAACTGCAGG TCCACCAGGA
2341 ACTCCAGGTC CACAGGGTTT GCTTGGAGCT CCTGGAATTC TTGGTTTACC TGGTTCAAGA
2401 GGAGAAAGAG GTCTTCCTGG TGTTGCTGGA GCCGTTGGAG AGCCAGGACC ATTGGGAATT
2461 GCTGGACCTC CAGGTGCTAG GGGACCACCT GGCGCTGTGG GATCTCCAGG CGTGAATGGT
2521 GCACCAGGAG AGGCAGGAAG GGATGGTAAT CCGGGTAACG ATGGACCTCC AGGTAGAGAT
2581 GGGCAACCAG GACACAAGGG GGAAAGGGGT TATCCAGGAA ATATTGGACC TGTTGGAGCT
2641 GCAGGTGCTC CAGGTCCTCA TGGACCAGTT GGACCTGCAG GAAAACATGG AAATAGAGGA
2701 GAGACTGGTC CTTCAGGTCC TGTGGGACCA GCAGGTGCTG TTGGTCCTAG AGGTCCATCA
2761 GGACCACAAG GTATTAGAGG AGATAAGGGA GAGCCAGGAG AAAAGGGACC AAGAGGTTTA
2821 CCTGGTTTGA AAGGACATAA TGGATTGCAG GGGCTTCCAG GTATTGCTGG CCATCACGGT
2881 GATCAGGGAG CTCCAGGTTC TGTAGGTCCA GCAGGTCCAA GGGGACCAGC TGGTCCTTCT
2941 GGACCTGCTG GTAAGGATGG TAGGACTGGT CATCCAGGAA CTGTGGGACC AGCAGGAATT
3001 AGGGGTCCTC AAGGGCATCA AGGTCCTGCT GGTCCACCTG GACCTCCAGG TCCTCCAGGA
3061 CCTCCTGGTG TTTCTGGTGG TGGGTATGAT TTTGGTTACG ATGGAGATTT TTATAGGGCA
3121 TGA
SEQ NO 5: Amino Acid Sequence of Spidroin-I
```

| | | | | | |
|---|---|---|---|---|---|
| 10 | 20 | 30 | 40 | 50 | 60 |
| QGAGAAAAAA | GGAGQGGYGG | LGGQGAGQGG | YGGLGGQGAG | QGAGAAAAAA | AGGAGQGGYG |
| 70 | 80 | 90 | 100 | 110 | 120 |
| GLGSQGAGRG | GQGAGAAAAA | AGGAGQGGYG | GLGSQGAGRG | GLGGQGAGAA | AAAAAGGAGQ |
| 130 | 140 | 150 | 160 | 170 | 180 |
| GGYGGLGNQG | AGRGGQGAAA | AAAGGAGQGG | YGGLGSQGAG | RGGLGGQGAG | AAAAAAGGAG |
| 190 | 200 | 210 | 220 | 230 | 240 |
| QGGYGGLGGQ | GAGQGGYGGL | GSQGAGRGGL | GGQGAGAAAA | AAAGGAGQGG | LGGQGAGQGA |
| 250 | 260 | 270 | 280 | 290 | 300 |
| GASAAAAGGA | GQGGYGGLGS | QGAGRGGEGA | GAAAAAAGGA | GQGGYGGLGG | QGAGQGGYGG |
| 310 | 320 | 330 | 340 | 350 | 360 |
| LGSQGAGRGG | LGGQGAGAAA | AGGAGQGGLG | GQGAGQGAGA | AAAAAGGAGQ | GGYGGLGSQG |
| 370 | 380 | 390 | 400 | 410 | 420 |
| AGRGGLGGQG | AGAVAAAAAG | GAGQGGYGGL | GSQGAGRGGQ | GAGAAAAAAG | GAGQRGYGGL |
| 430 | 440 | 450 | 460 | 470 | 480 |
| GNQGAGRGGL | GGQGAGAAAA | AAAGGAGQGG | YGGLGNQGAG | RGGQGAAAAA | GGAGQGGYGG |
| 490 | 500 | 510 | 520 | 530 | 540 |
| LGSQGAGRGG | QGAGAAAAAA | VGAGQEGIRG | QGAGQGGYGG | LGSQGSGRGG | LGGQGAGAAA |
| 550 | 560 | 570 | 580 | 590 | 600 |
| AAAGGAGQGG | LGGQGAGQGA | GAAAAAAGGV | RQGGYGGLGS | QGAGRGGQGA | GAAAAAAGGA |
| 610 | 620 | 630 | 640 | 650 | 660 |
| GQGGYGGLGG | QGVGRGGLGG | QGAGAAAAGG | AGQGGYGGVG | SGASAASAAA | SRLSSPQASS |
| 670 | 680 | 690 | 700 | 710 | 720 |
| RVSSAVSNLV | ASGPTNSAAL | SSTISNVVSQ | IGASNPGLSG | CDVLIQALLE | VVSALIQILG |
| 730 | 740 | | | | |
| SSSIGQVNYG | SAGQATQIVG | QSVYQALG | | | |

```
SEQ NO 6: Nucleotide Sequence of Spidroin-I, Codon Optimized for
Nicotiana Benthamiana Chloroplast Expression
      1 CAAGGAGCTG GAGCTGCAGC TGCAGCTGCT GGAGGAGCTG GACAAGGTGG ATATGGTGGT
    61 TTGGGAGGTC AAGGTGCTGG ACAAGGAGGT TACGGAGGTC TTGGAGGACA GGGAGCTGGA
  121 CAAGGTGCTG GTGCTGCAGC TGCTGCTGCT GCTGGAGGAG CTGGTCAAGG AGGTTACGGT
  181 GGTTTAGGTT CACAAGGAGC TGGTAGAGGA GGACAGGGTG CAGGTGCTGC TGCTGCAGCA
  241 GCAGGAGGTG CTGGACAGGG CGGTTATGGT GGTTTGGGTT CCCAAGGAGC TGGAAGGGGA
  301 GGATTAGGTG GACAGGGAGC TGGTGCTGCT GCTGCTGCTG CTGCAGGAGG TGCTGGTCAG
  361 GGTGGCTATG GTGGTTTGGG GAATCAAGGA GCTGGAAGGG GTGGTCAAGG AGCTGCAGCA
  421 GCTGCTGCTG GAGGTGCTGG ACAAGGCGGT TACGGAGGTT TGGGATCTCA GGGTGCAGGA
  481 AGAGGAGGTC TTGGCGGACA GGGAGCTGGA GCTGCTGCCG CTGCTGCTGG AGGTGCAGGA
  541 CAAGGCGGTT ATGGTGGTCT TGGTGGTCAA GGAGCAGGAC AAGGTGGGTA CGGGGGACTT
  601 GGAAGTCAGG GTGCTGGTAG GGGAGGACTT GGAGGACAAG GTGCTGGGGC AGCTGCTGCA
  661 GCTGCTGCTG GAGGTGCTGG ACAAGGAGGT CTTGGTGGAC AGGGAGCAGG ACAAGGTGCT
  721 GGAGCATCTG CAGCTGCAGC AGGTGGAGCA GGGCAGGGAG GATACGGAGG ACTTGGTTCT
  781 CAAGGTGCTG GTAGAGGAGG TGAAGGAGCA GGTGCTGCTG CAGCTGCAGC TGGAGGCGCT
  841 GGACAAGGAG GCTATGGAGG ACTTGGTGGA CAGGGGGCTG GACAGGGTGG TTATGGAGGT
  901 CTTGGATCCC AGGGAGCTGG GAGGGGAGGA CTCGGAGGAC AAGGAGCTGG AGCTGCAGCT
  961 GCTGGTGGTG CTGGACAAGG TGGACTTGGT GGACAAGGAG CTGGCCAAGG TGCTGGAGCT
1021 GCAGCTGCTG CTGCTGGTGG TGCAGGTCAA GGAGGATATG GAGGACTTGG AAGTCAAGGA
1081 GCTGGAAGAG GAGGTCTTGG TGGTCAAGGT GCCGGAGCAG TTGCTGCCGC TGCTGCTGGA
1141 GGTGCTGGTC AGGGTGGATA TGGAGGTCTT GGTTCTCAGG GTGCAGGAAG AGGAGGTCAA
1201 GGAGCAGGTG CTGCAGCTGC AGCTGCAGGT GGAGCTGGTC AAAGAGGATA CGGAGGACTT
1261 GGAAACCAGG GTGCTGGTAG AGGAGGCCTT GGAGGTCAGG GAGCTGGGGC TGCTGCTGCT
1321 GCTGCAGCTG GTGGTGCTGG ACAAGGTGGA TATGGTGGAC TCGGAAATCA GGGTGCTGGA
1381 AGAGGTGGAC AAGGTGCTGC AGCTGCAGCT GGCGGAGCAG GACAAGGTGG ATATGGTGGT
1441 TTGGGTTCAC AGGGAGCAGG AAGAGGAGGA CAGGGAGCTG GTGCTGCTGC TGCAGCAGCT
1501 GTTGGAGCAG GACAAGAAGG AATTAGAGGA CAAGGAGCTG GTCAGGGAGG ATATGGAGGT
1561 TTGGGATCTC AAGGTTCAGG TAGGGGTGGA CTTGGTGGAC AAGGAGCAGG AGCAGCTGCT
1621 GCTGCTGCTG GTGGAGCTGG ACAAGGAGGA TTGGGAGGAC AAGGAGCTGG TCAAGGAGCT
1681 GGAGCTGCTG CCGCAGCTGC TGGAGGAGTG AGACAAGGTG GGTATGGTGG TTTGGGTTCA
1741 CAAGGAGCAG GTAGAGGTGG ACAAGGAGCA GGTGCAGCAG CAGCAGCTGC TGGTGGTGCT
1801 GGTCAAGGAG GTTACGGTGG ACTTGGAGGT CAAGGAGTTG GTAGGGGAGG TCTTGGTGGT
1861 CAAGGGGCTG GTGCTGCTGC CGCTGGTGGA GCTGGACAAG GTGGTTATGG TGGTGTTGGT
1921 TCTGGAGCTT CTGCTGCTAG TGCTGCTGCA TCAAGACTTT CTTCTCCTCA AGCTTCTTCT
1981 AGAGTTAGCA GCGCTGTTAG TAACCTTGTA GCTTCAGGTC CAACTAATTC TGCTGCTCTT
2041 TCTAGTACTA TTTCTAATGT TGTGAGCCAA ATCGGAGCTT CAAATCCAGG ATTGTCTGGT
2101 TGTGATGTTT TAATTCAAGC TTTGTTGGAG GTGGTTTCTG CTCTTATTCA GATTTTGGGT
2161 TCTTCTTCTA TCGGTCAAGT TAACTATGGA TCTGCTGGTC AGGCTACACA AATTGTTGGT
2221 CAGTCTGTTT ACCAGGCTCT TGGGTGA
SEQ NO 7: Amino Acid Sequence of Fibroin-III
```

| | | | | | |
|---|---|---|---|---|---|
| 10 | 20 | 30 | 40 | 50 | 60 |
| ARAGSGQQGP | GQQGPGQQGP | GQQGPYGPGA | SAAAAAAGGY | GPGSGQQGPS | QQGPGQQGPG |
| 70 | 80 | 90 | 100 | 110 | 120 |
| GQGPYGPGAS | AAAAAAGGYG | PGSGQQGPGG | QGPYGPGSSA | AAAAAGGNGP | GSGQQGAGQQ |
| 130 | 140 | 150 | 160 | 170 | 180 |
| GPGQQGPGAS | AAAAAAGGYG | PGSGQQGPGQ | QGPGGQGPYG | PGASAAAAAA | GGYGPGSGQG |
| 190 | 200 | 210 | 220 | 230 | 240 |
| PGQQGPGGQG | PYGPGASAAA | AAAGGYGPGS | GQQGPGQQGP | GQQGPGGQGP | YGPGASAAAA |
| 250 | 260 | 270 | 280 | 290 | 300 |
| AAGGYGPGYG | QQGPGQQGPG | GQGPYGPGAS | AASAASGGYG | PGSGQQGPGQ | QGPGGQGPYG |
| 310 | 320 | 330 | 340 | 350 | 360 |
| PGASAAAAAA | GGYGPGSGQQ | GPGQQGPGQQ | GPGQQGPGGQ | GPYGPGASAA | AAAAGGYGPG |
| 370 | 380 | 390 | 400 | 410 | 420 |
| SGQQGPGQQG | PGQQGPGQQG | PGQQGPGQQG | PGQQGPGQQG | PGQQGPGGQG | AYGPGASAAA |
| 430 | 440 | 450 | 460 | 470 | 480 |
| GAAGGYGPGS | GQQGPGQQGP | GQQGPGQQGP | GQQGPGQQGP | GQQGPGQQGP | YGPGASAAAA |
| 490 | 500 | 510 | 520 | 530 | 540 |
| AAGGYGPGSG | QQGPGQQGPG | QQGPGGQGPY | GPGAASAAVS | VGGYGPQSSS | VPVASAVASR |
| 550 | 560 | 570 | 580 | 590 | 600 |
| LSSPAASSRV | SSAVSSLVSS | GPTKHAALSN | TISSVVSQVS | ASNPGLSGCD | VLVQALLEVV |
| 610 | 620 | 630 | | | |
| SALVSILGSS | SIGQINYGAS | AQYTQMVGQS | VAQALA | | |

```
SEQ NO 8: Nucleotide Sequence of Fibroin-IIII, Codon Optimized for
Nicotiana Benthamiana Chloroplast Expression
      1 GCAAGAGCAG GATCTGGTCA GCAAGGACCT GGTCAACAAG GTCCAGGACA GCAGGGACCA
    61 GGACAACAAG GTCCTTATGG ACCTGGTGCT TCAGCAGCTG CTGCTGCTGC TGGTGGTTAT
  121 GGACCAGGAT CTGGACAACA GGGCCCTTCT CAGCAAGGAC CTGGACAGCA GGGTCCTGGT
  181 GGTCAAGGAC CTTACGGTCC TGGAGCTTCT GCTGCTGCTG CTGCTGCAGG TGGATATGGT
  241 CCTGGTAGTG GTCAACAAGG TCCAGGAGGA CAAGGACCAT ACGGCCCTGG TAGCTCAGCT
  301 GCTGCAGCAG CTGCTGGTGG TAATGGCCCA GGAAGTGGAC AACAAGGTGC TGGACAACAG
  361 GGTCCAGGAC AACAAGGACC TGGTGCATCA GCCGCTGCTG CTGCTGCTGG AGGTTATGGA
  421 CCAGGTTCTG GACAACAAGG TCCAGGACAA CAAGGACCAG GAGGTCAAGG ACCTTATGGA
  481 CCAGGAGCTA GTGCTGCAGC AGCTGCTGCT GGAGGTTATG GTCCTGGAAG TGGTCAAGGA
  541 CCTGGACAAC AAGGACCTGG GGGTCAAGGT CCTTATGGAC CTGGAGCTTC CGCTGCTGCT
  601 GCAGCAGCCG GAGGTTATGG TCCAGGAAGT GGTCAGCAAG GACCAGGACA GCAGGGACCA
  661 GGACAGCAAG GTCCTGGAGG TCAGGGACCA TATGGTCCAG GAGCTTCTGC TGCTGCTGCA
  721 GCTGCTGGTG GATATGGACC AGGTTATGGA CAACAAGGAC CTGGACAACA AGGACCTGGA
  781 GGGCAAGGTC CATATGGACC AGGAGCTTCT GCTGCTAGTG CTGCTTCAGG AGGGTATGGA
  841 CCAGGTTCTG GACAACAAGG ACCAGGACAA CAAGGTCCAG GTGGTCAAGG ACCTTATGGA
  901 CCGGGTGCTT CTGCAGCTGC CGCAGCTGCT GGAGGATATG GTCCTGGTTC AGGTCAACAA
  961 GGACCCGGTC AACAAGGACC AGGTCAGCAA GGTCCAGGAC AACAGGGTCC TGGAGGTCAG
1021 GGTCCTTATG GGCCAGGAGC ATCTGCTGCA GCTGCTGCTG CTGGTGGTTA TGGACCAGGC
1081 TCTGGACAAC AAGGACCTGG TCAACAAGGA CCTGGACAGC AGGGTCCTGG ACAGCAAGGT
1141 CCAGGACAAC AAGGACCAGG ACAGCAAGGT CCAGGTCAGC AAGGACCTGG ACAACAAGGA
1201 CCAGGTCAAC AAGGACCTGG TGGTCAGGGT GCTTATGGTC CAGGTGCTAG TGCTGCTGCC
1261 GGAGCAGCTG GAGGCTATGG ACCTGGATCT GGTCAGCAGG GACCTGGTCA ACAAGGACCT
1321 GGTCAACAGG GACCAGGTCA ACAAGGACCA GGGCAACAGG GACCTGGACA ACAAGGACCT
1381 GGACAACAAG GTCCAGGTCA GCAGGGACCT TATGGACCTG GTGCTTCAGC TGCTGCTGCA
1441 GCTGCTGGAG GATATGGCCC AGGTTCTGGA CAACAGGGAC CTGGACAACA AGGTCCAGGT
1501 CAACAAGGTC CAGGTGGCCA AGGACCATAC GGACCAGGAG CTGCATCTGC AGCTGTTTCT
1561 GTTGGAGGAT ATGGACCTCA ATCATCATCT GTTCCTGTGG CATCAGCTGT TGCTTCAAGA
1621 TTGTCTTCAC CAGCTGCTTC ATCTAGAGTT AGCAGTGCAG TTTCTTCCTT GGTTTCTTCT
1681 GGACCTACTA AACATGCTGC TCTCTCTAAT ACTATTAGTA GTGTTGTTTC TCAGGTTTCT
1741 GCTTCTAATC CAGGTTTATC AGGATGCGAT GTTCTTGTTC AAGCACTTTT GGAAGTTGTT
1801 TCCGCTTTGG TTTCAATTTT AGGATCATCT TCTATTGGTC AAATTAATTA TGGCGCTTCT
1861 GCCCAATATA CTCAGATGGT CGGACAATCA GTTGCTCAAG CTCTTGCTTA A
SEQ NO 9: Amino Acid Sequence of P4H Alpha Subunit
```

| | | | | | |
|---|---|---|---|---|---|
| 10 | 20 | 30 | 40 | 50 | 60 |
| MDISNLPPHI | RQQILGLISK | PQQNNDESSS | SNNKNNLINN | EKVSNVLIDL | TSNLKIENFK |
| 70 | 80 | 90 | 100 | 110 | 120 |
| IFNKESLNQL | EKKGYLIIDN | FLNDLNKINL | IYDESYNQFK | ENKLIEAGMN | KGTDKWKDKS |
| 130 | 140 | 150 | 160 | 170 | 180 |
| IRGDYIQWIH | RDSNSRIQDK | DLSSTIRNIN | YLLDKLDLIK | NEFDNVIPNF | NSIKTQTQLA |
| 190 | 200 | 210 | 220 | 230 | 240 |
| VYLNGGRYIK | HRDSFYSSES | LTISRRITMI | YYVNKDWKKG | DGGELRLYTN | NPNNTNQKEL |
| 250 | 260 | 270 | 280 | | |
| KQTEEFIDIE | PIADRLLIFL | SPFLEHEVLQ | CNFEPRIAIT | TWIY | |

SEQ NO 10: Amino Acid Sequence of P4H Beta Subunit

| | | | | | |
|---|---|---|---|---|---|
| 10 | 20 | 30 | 40 | 50 | 60 |
| APDEEDHVLV | LHKGNFDEAL | AAHKYLLVEF | YAPWCGHCKA | LAPEYAKAAG | KLKAEGSEIR |
| 70 | 80 | 90 | 100 | 110 | 120 |
| LAKVDATEES | DLAQQYGVRG | YPTIKFFKNG | DTASPKEYTA | GREADDIVNW | LKKRTGPAAS |
| 130 | 140 | 150 | 160 | 170 | 180 |
| TLSDGAAAEA | LVESSEVAVI | GFFKDMESDS | AKQFFLAAEV | IDDIPFGITS | NSDVESKYQL |
| 190 | 200 | 210 | 220 | 230 | 240 |
| DKDGVVLFKK | FDEGRNNFEG | EVTKEKLLDF | IKHNQLPLVI | EFTEQTAPKI | FGGEIKTHIL |
| 250 | 260 | 270 | 280 | 290 | 300 |
| LFLPKSVSDY | EGKLSNFKKA | AESFKGKILF | IFIDSDHTDN | QRILEFFGLK | KEECPAVRLI |
| 310 | 320 | 330 | 340 | 350 | 360 |
| TLEEEMTKYK | PESDELTAEK | ITEFCHRFLE | GKIKPHLMSQ | ELPDDWDKQP | VKVLVGKNFE |
| 370 | 380 | 390 | 400 | 410 | 420 |
| EVAFDEKKNV | FVEFYAPWCG | HCKQLAPIWD | KLGETYKDHE | NIVIAKMDST | ANEVEAVKVH |
| 430 | 440 | 450 | 460 | 470 | 480 |
| SFPTLKFFPA | SADRTVIDYN | GERTLDGFKK | FLESGGQDGA | GDDDDLEDLE | EAEEPDLEED |
| 490 | | | | | |
| DDQKAVKDEL | | | | | |

```
SEQ NO 11: Nucleotide Sequence of P4H Alpha Subunit, Codon Optimized
for Nicotiana Benthamiana Chloroplast Expression
    1 ATGGATATTT CTAATTTGCC ACCACATATC AGGCAGCAAA TTCTTGGCTT GATTTCTAAG
  61 CCACAACAAA ATAACGATGA GAGTTCTTCA TCAAACAATA AAAATAACCT TATTAACAAC
121 GAGAAGGTGT CAAATGTTTT GATTGATCTT ACTTCCAATC TTAAGATTGA GAACTTCAAA
181 ATCTTTAACA AGGAGTCTCT CAATCAGCTT GAAAAGAAGG GATACCTTAT TATTGATAAT
241 TTCTTGAATG ATTTGAATAA AATTAATTTA ATTTATGATG AATCCTATAA TCAATTTAAA
301 GAAAATAAAC TTATAGAAGC TGGAATGAAC AAGGGAACTG ATAAGTGGAA AGATAAGTCA
361 ATCAGAGGAG ATTACATACA GTGGATTCAT CGGGATTCTA ATAGTAGAAT TCAGGATAAG
421 GATCTTTCAT CTACGATTAG AAATATTAAT TATCTTCTCG ATAAACTTGA CTTGATTAAA
481 AATGAGTTTG ATAATGTCAT TCCAAATTTT AATTCAATCA AAACACAAAC TCAATTAGCA
541 GTGTACTTGA ATGGCGGTAG GTACATTAAG CATAGAGATT CTTTCTATTC TTCTGAAAGT
601 CTTACTATTT CTAGGAGGAT TACTATGATA TACTATGTGA ATAAGGATTG GAAGAAGGGA
661 GATGGAGGTG AGTTGAGATT GTATACAAAC AATCCAAATA ATACAAATCA AAAAGAGTTG
721 AAGCAGACTG AAGAATTCAT TGATATTGAA CCTATTGCTG ATCGTTTACT TATTTTTCTT
781 TCCCCCTTCC TTGAACATGA GGTGCTTCAA TGCAATTTCG AGCCAAGGAT AGCTATTACT
841 ACTTGGATAT ACTAA
SEQ NO 12: Nucleotide Sequence of P4H Beta Subunit, Codon Optimized
for Nicotiana Benthamiana Chloroplast Expression
      1 GCTCCTGATG AAGAGGACCA TGTACTTGTT TTGCATAAGG GTAATTTTGA TGAAGCTCTT
    61 GCAGCACATA AATATCTATT GGTGGAGTTC TATGCACCTT GGTGTGGACA CTGCAAGGCT
  121 TTGGCTCCAG AGTACGCTAA GGCTGCAGGA AAACTTAAGG CTGAGGGTTC TGAGATTAGA
  181 CTCGCTAAGG TTGATGCTAC TGAGGAATCA GATTTGGCTC AGCAATATGG AGTTAGGGGA
  241 TACCCAACTA TTAAATTTTT CAAAAATGGG GATACTGCAT CACCAAAAGA ATACACTGCC
  301 GGGAGAGAAG CTGATGATAT TGTTAATTGG TTGAAGAAGA GGACTGGCCC TGCTGCTTCT
  361 ACTTTGTCTG ATGGAGCAGC TGCAGAGGCA CTTGTTGAAT CTTCAGAAGT TGCTGTTATT
  421 GGATTTTTCA AAGATATGGA GTCCGATTCT GCTAAGCAAT TCTTTCTGGC AGCAGAAGTC
  481 ATTGATGATA TTCCATTTGG AATCACTTCA AATTCTGATG TTTTTTCTAA GTATCAGCTT
  541 GATAAGGATG GGGTTGTTCT TTTTAAAAAA TTCGACGAGG GAAGGAATAA CTTCGAGGGA
  601 GAGGTGACAA AAGAGAAGCT TCTTGATTTC ATTAAGCATA ATCAGCTCCC TCTTGTTATT
  661 GAATTTACCG AACAGACTGC ACCTAAGATC TTCGGTGGAG AAATTAAAAC TCATATTTTG
  721 CTTTTTTTGC CAAAATCTGT TTCAGATTAT GAAGGAAAAC TTTCTAATTT TAAGAAAGCT
  781 GCTGAATCAT TTAAGGGTAA GATTTTGTTT ATTTTTATTG ACTCAGATCA TACTGATAAT
  841 CAAAGGATCT TGGAATTTTT TGGGTTAAAG AAGGAGGAAT GTCCTGCTGT TAGACTTATT
  901 ACTTTGGAGG AAGAAATGAC AAAGTACAAG CCTGAAAGTG ATGAATTGAC AGCAGAAAAG
  961 ATTACTGAAT TCTGTCATCG TTTCCTGGAA GGTAAGATTA AGCCACATTT GATGTCCCAA
1021 GAACTTCCTG ATGATTGGGA TAAGCAACCT GTTAAGGTTC TCGTTGGTAA GAATTTTGAA
1081 GAGGTGGCTT TTGATGAGAA AAAGAATGTC TTTGTTGAGT TCTATGCACC TTGGTGTGGA
1141 CACTGTAAAC AATTGGCTCC AATTTGGGAC AAGCTCGGTG AAACCTATAA GGATCACGAA
1201 AACATTGTTA TTGCTAAGAT GGATTCTACA GCTAATGAAG TGGAGGCTGT TAAGGTGCAT
1261 TCTTTTCCAA CACTTAAATT TTTCCCAGCT TCAGCTGATA GGACTGTAAT AGATTATAAC
1321 GGAGAGCGTA CTCTAGATGG TTTCAAAAAG TTTCTGGAGT CAGGTGGTCA AGATGGAGCT
1381 GGAGATGATG ATGATCTTGA AGATCTCGAG GAAGCTGAGG AACCAGATCT TGAGGAGGAT
1441 GATGATCAAA AAGCTGTTAA GGATGAGTTG TGA
SEQ NO 13: Amino Acid Sequence of LH3
```

| | | | | | |
|---|---|---|---|---|---|
| 10 | 20 | 30 | 40 | 50 | 60 |
| SDRPRGRDPV | NPEKLLVITV | ATAETEGYLR | FLRSAEFFNY | TVRTLGLGEE | WRGGDVARTV |
| 70 | 80 | 90 | 100 | 110 | 120 |
| GGGQKVRWLK | KEMEKYADRE | DMIIMFVDSY | DVILAGSPTE | LLKKFVQSGS | RLLFSAESFC |
| 130 | 140 | 150 | 160 | 170 | 180 |
| WPEWGLAEQY | PEVGTGKRFL | NSGGFIGFAT | TIHQIVRQWK | YKDDDDDQLF | YTRLYLDPGL |
| 190 | 200 | 210 | 220 | 230 | 240 |
| REKLSLNLDH | KSRIFQNLNG | ALDEVVLKED | RNRVRIRNVA | YDTLPIVVHG | NGPTKLQLNY |
| 250 | 260 | 270 | 280 | 290 | 300 |
| LGNYVPNGWT | PEGGCGFCNQ | DRRTLPGGQP | PPRVFLAVFV | EQPTPFLPRF | LQRLLLLDYP |
| 310 | 320 | 330 | 340 | 350 | 360 |
| PDRVTLFLHN | NEVFHEPHIA | DSWPQLQDHF | SAVKLVGPEE | ALSPGEARDM | AMDLCRQDPE |
| 370 | 380 | 390 | 400 | 410 | 420 |
| CEFYFSLDAD | AVLTNLQTLR | ILIEENRKVI | APMLSRHGKL | WSNFWGALSP | DEYYARSEDY |
| 430 | 440 | 450 | 460 | 470 | 480 |
| VELVQRKRVG | VWNVPYISQA | YVIRGDTLRM | ELPQRDVFSG | SDTDPDMAFC | KSFRDKGIFL |
| 490 | 500 | 510 | 520 | 530 | 540 |
| HLSNQHEFGR | LLATSRYDTE | HLHPDLWQIF | DNPVDWKEQY | IHENYSRALE | GEGIVEQPCP |
| 550 | 560 | 570 | 580 | 590 | 600 |
| DVYWFPLLSE | QMCDELVAEM | EHYGQWSGGR | HEDSRLAGGY | ENVPTVDIHM | KQVGYEDQWL |
| 610 | 620 | 630 | 640 | 650 | 660 |
| QLLRTYVGPM | TESLFPGYHT | KARAVMNFVV | RYRPDEQPSL | RPHHDSSTFT | LNVALNHKGL |
| 670 | 680 | 690 | 700 | 710 | |
| DYEGGGCRFL | RYDCVISSPR | KGWALLHPGR | LTHYHEGLPT | TWGTRYIMVS | FVDP |

```
SEQ NO 14: Nucleotide Sequence of LH3, Codon Optimized for Nicotiana
Benthamiana Chloroplast Expression
      1 TCTGATAGGC CACGTGGAAG AGATCCTGTG AACCCAGAGA AGCTTTTGGT TATTACTGTT
    61 GCTACTGCAG AGACTGAAGG ATACCTTAGA TTTCTTCGTT CAGCAGAGTT CTTTAATTAT
  121 ACTGTTAGAA CTTTGGGATT GGGTGAAGAA TGGAGAGGAG GAGATGTTGC TAGGACTGTT
  181 GGTGGTGGAC AAAAAGTTAG GTGGTTGAAG AAGGAAATGG AAAAGTATGC TGATAGGGAG
  241 GATATGATTA TTATGTTTGT GGATTCCTAT GACGTTATTC TTGCTGGATC TCCTACCGAG
  301 CTTCTTAAAA AGTTTGTTCA ATCAGGATCA AGACTTCTCT TTAGTGCAGA GAGTTTCTGT
  361 TGGCCAGAAT GGGGTCTCGC TGAACAATAC CCAGAAGTTG GAACTGGAAA GAGATTCTTG
  421 AATTCTGGAG GATTTATTGG ATTTGCTACA ACTATTCATC AAATTGTTAG ACAGTGGAAG
  481 TATAAGGATG ATGATGATGA TCAGCTTTTT TATACTAGGC TTTATTTAGA TCCTGGTCTC
  541 AGAGAAAAAC TTTCTTTGAA TCTTGATCAT AAGAGCAGAA TCTTCCAAAA TCTTAACGGA
  601 GCTTTGGATG AGGTTGTTCT TAAATTTGAT AGAAACAGAG TTAGGATTAG AAATGTTGCT
  661 TATGATACTT TGCCTATTGT TGTTCATGGA AATGGACCAA CTAAGCTTCA ACTTAACTAT
  721 CTTGGTAACT ACGTTCCTAA TGGATGGACT CCAGAAGGTG GTTGTGGATT TTGCAACCAG
  781 GATAGAAGGA CACTTCCTGG GGGACAACCC CCTCCAAGGG TGTTCTTGGC TGTTTTTGTT
  841 GAACAACCAA CTCCATTTTT GCCTCGTTTT TTGCAAAGAC TGCTACTTCT TGATTATCCA
  901 CCTGATAGAG TTACTTTGTT CTTGCATAAT AATGAAGTTT TTCATGAACC ACATATTGCA
  961 GATTCTTGGC CACAACTCCA AGATCACTTT AGCGCTGTTA AACTTGTTGG TCCAGAAGAA
1021 GCTTTGTCTC CTGGTGAAGC AAGAGACATG GCCATGGATC TCTGCAGACA AGACCCAGAA
1081 TGTGAATTTT ATTTTAGTTT GGATGCTGAT GCTGTTTTGA CTAATTTGCA GACATTGAGA
1141 ATTCTTATCG AAGAAAACAG GAAAGTTATT GCTCCCATGC TTTCAAGGCA TGGAAAGCTC
1201 TGGTCTAATT TCTGGGGTGC ACTTTCTCCA GACGAATACT ATGCAAGATC TGAAGATTAT
1261 GTTGAGTTGG TTCAGAGAAA GAGAGTTGGA GTTTGGAACG TTCCCTATAT TTCACAGGCT
1321 TACGTTATTA GAGGAGATAC CTTGCGTATG GAATTGCCAC AGAGGGATGT TTTCTCTGGC
1381 TCTGATACAG ATCCAGATAT GGCTTTCTGC AAATCTTTTA GAGACAAAGG TATTTTTTTG
1441 CATTTGTCAA ACCAACATGA ATTCGGTAGA CTTCTTGCTA CCTCTAGGTA TGATACTGAA
1501 CACCTTCATC CAGATCTCTG GCAAATTTTT GACAATCCTG TTGACTGGAA GGAGCAATAT
1561 ATTCACGAGA ACTACTCTAG AGCTCTTGAA GGAGAGGGTA TTGTTGAGCA ACCTTGCCCT
1621 GATGTGTACT GGTTTCCTTT GCTCTCTGAG CAAATGTGTG ATGAACTTGT TGCTGAAATG
1681 GAACATTACG GCCAATGGTC AGGTGGTAGG CATGAGGATT CAAGACTTGC TGGTGGTTAC
1741 GAAAACGTTC CAACAGTGGA TATCCATATG AAGCAAGTTG GTTATGAAGA TCAGTGGCTT
1801 CAATTGCTTA GGACTTATGT TGGACCTATG ACTGAATCAC TTTTTCCAGG ATATCACACA
1861 AAGGCTAGAG CTGTGATGAA TTTTGTTGTT AGATATAGAC CAGATGAACA GCCATCATTG
1921 AGACCACATC ATGATTCTTC TACTTTTACT TTAAATGTGG CTCTTAACCA TAAGGGACTG
1981 GATTACGAAG GAGGAGGTTG CAGATTTCTT AGATATGATT GTGTTATTTC CTCTCCAAGA
2041 AAAGGGTGGG CATTGTTACA TCCAGGTAGA CTTACACATT ACCATGAAGG ACTTCCTACT
2101 ACTTGGGGAA CTCGTTATAT TATGGTTTCT TTCGTTGATC CTTAA

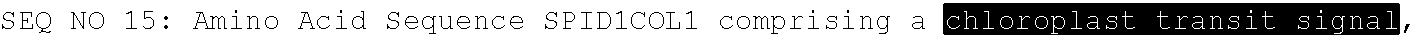

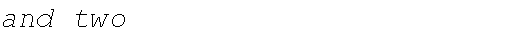

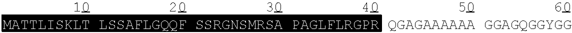



```

| | | | | | |
|---|---|---|---|---|---|
| 70 | 80 | 90 | 100 | 110 | 120 |
| LGGQGAGQGG | YGGLGGQGAG | QGAGAAAAAA | AGGAGQGGYG | GLGSQGAGRG | GQGAGAAAAA |
| 130 | 140 | 150 | 160 | 170 | 180 |
| AGGAGQGGYG | GLGSQGAGRG | GLGGQGAGAA | AAAAAGGAGQ | GGYGGLGNQG | AGRGGQGAAA |
| 190 | 200 | 210 | 220 | 230 | 240 |
| AAAGGAGQGG | YGGLGSQGAG | RGGLGGQGAG | AAAAAAGGAG | QGGYGGLGGQ | GAGQGGYGGL |
| 250 | 260 | 270 | 280 | 290 | 300 |
| GSQGAGRGGL | GGQGAGAAAA | AAAGGAGQGG | LGGQGAGQGA | GASAAAAGGA | GQGGYGGLGS |
| 310 | 320 | 330 | 340 | 350 | 360 |
| QGAGRGGEGA | GAAAAAAGGA | GQGGYGGLGG | QGAGQGGYGG | LGSQGAGRGG | LGGQGAGAAA |
| 370 | 380 | 390 | 400 | 410 | 420 |
| AGGAGQGGLG | GQGAGQGAGA | AAAAAGGAGQ | GGYGGLGSQG | AGRGGLGGQG | AGAVAAAAAG |
| 430 | 440 | 450 | 460 | 470 | 480 |
| GAGQGGYGGL | GSQGAGRGGQ | GAGAAAAAAG | GAGQRGYGGL | GNQGAGRGGL | GGQGAGAAAA |
| 490 | 500 | 510 | 520 | 530 | 540 |
| AAAGGAGQGG | YGGLGNQGAG | RGGQGAAAAA | GGAGQGGYGG | LGSQGAGRGG | QGAGAAAAAA |
| 550 | 560 | 570 | 580 | 590 | 600 |
| VGAGQEGIRG | QGAGQGGYGG | LGSQGSGRGG | LGGQGAGAAA | AAAGGAGQGG | LGGQGAGQGA |
| 610 | 620 | 630 | 640 | 650 | 660 |
| GAAAAAAGGV | RQGGYGGLGS | QGAGRGGQGA | GAAAAAAGGA | GQGGYGGLGG | QGVGRGGLGG |
| 670 | 680 | 690 | 700 | 710 | 720 |
| QGAGAAAAGG | AGQGGYGGVG | SGASAASAAA | SRLSSPQASS | RVSSAVSNLV | ASGPTNSAAL |
| 730 | 740 | 750 | 760 | 770 | 780 |
| SSTISNVVSQ | IGASNPGLSG | CDVLIQALLE | VVSALIQILG | SSSIGQVNYG | SAGQATQIVG |

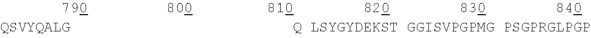

| | | | | | |
|---|---|---|---|---|---|
| 850 | 860 | 870 | 880 | 890 | 900 |
| PGAPGPQGFQ | GPPGEPGEPG | ASGPMGPRGP | PGPPGKNGDD | GEAGKPGRPG | ERGPPGPQGA |
| 910 | 920 | 930 | 940 | 950 | 960 |
| RGLPGTAGLP | GMKGHRGFSG | LDGAKGDAGP | AGPKGEPGSP | GENGAPGQMG | PRGLPGERGR |
| 970 | 980 | 990 | 1000 | 1010 | 1020 |
| PGAPGPAGAR | GNDGATGAAG | PPGPTGPAGP | PGFPGAVGAK | GEAGPQGPRG | SEGPQGVRGE |
| 1030 | 1040 | 1050 | 1060 | 1070 | 1080 |
| PGPPGPAGAA | GPAGNPGADG | QPGAKGANGA | PGIAGAPGFP | GARGPSGPQG | PGGPPGPKGN |
| 1090 | 1100 | 1110 | 1120 | 1130 | 1140 |
| SGEPGAPGSK | GDTGAKGEPG | PVGVQGPPGP | AGEEGKRGAR | GEPGPTGLPG | PPGERGGPGS |
| 1150 | 1160 | 1170 | 1180 | 1190 | 1200 |
| RGFPGADGVA | GPKGPAGERG | SPGPAGPKGS | PGEAGRPGEA | GLPGAKGLTG | SPGSPGPDGK |
| 1210 | 1220 | 1230 | 1240 | 1250 | 1260 |
| TGPPGPAGQD | GRPGPPGPPG | ARGQAGVMGF | PGPKGAAGEP | GKAGERGVPG | PPGAVGPAGK |
| 1270 | 1280 | 1290 | 1300 | 1310 | 1320 |
| DGEAGAQGPP | GPAGPAGERG | EQGPAGSPGF | QGLPGPAGPP | GEAGKPGEQG | VPGDLGAPGP |
| 1330 | 1340 | 1350 | 1360 | 1370 | 1380 |
| SGARGERGFP | GERGVQGPPG | PAGPRGANGA | PGNDGAKGDA | GAPGAPGSQG | APGLQGMPGE |
| 1390 | 1400 | 1410 | 1420 | 1430 | 1440 |
| RGAAGLPGPK | GDRGDAGPKG | ADGSPGKDGV | RGLTGPIGPP | GPAGAPGDKG | ESGPSGPAGP |
| 1450 | 1460 | 1470 | 1480 | 1490 | 1500 |
| TGARGAPGDR | GEPGPPGPAG | FAGPPGADGQ | PGAKGEPGDA | GAKGDAGPPG | PAGPAGPPGP |
| 1510 | 1520 | 1530 | 1540 | 1550 | 1560 |
| IGNVGAPGAK | GARGSAGPPG | ATGFPGAAGR | VGPPGPSGNA | GPPGPPGPAG | KEGGKGPRGE |
| 1570 | 1580 | 1590 | 1600 | 1610 | 1620 |
| TGPAGRPGEV | GPPGPPGPAG | EKGSPGADGP | AGAPGTPGPQ | GIAGQRGVVG | LPGQRGERGE |
| 1630 | 1640 | 1650 | 1660 | 1670 | 1680 |
| PGLPGPSGEP | GKQGPSGASG | ERGPPGPMGP | PGLAGPPGES | GREGAPGAEG | SPGRDGSPGA |
| 1690 | 1700 | 1710 | 1720 | 1730 | 1740 |
| KGDRGETGPA | GPPGAPGAPG | APGPVGPAGK | SGDRGETGPA | GPAGPVGPVG | ARGPAGPQGP |
| 1750 | 1760 | 1770 | 1780 | 1790 | 1800 |
| RGDKGETGEQ | GDRGIKGHRG | FSGLQGPPGP | PGSPGEQGPS | GASGPAGPRG | PPGSAGAPGK |
| 1810 | 1820 | 1830 | 1840 | 1850 | 1860 |
| DGLNGLPGPI | GPPGPRGRTG | DAGPVGPPGP | PGPPGPPGPP | SAGFDFSFLP | QPPQEKAHDG |

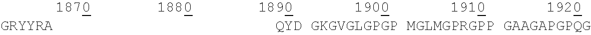

| | | | | | |
|---|---|---|---|---|---|
| 1930 | 1940 | 1950 | 1960 | 1970 | 1980 |
| FQGPAGEPGE | PGQTGPAGAR | GPAGPPGKAG | EDGHPGKPGR | PGERGVVGPQ | GARGFPGTPG |
| 1990 | 2000 | 2010 | 2020 | 2030 | 2040 |
| LPGFKGIRGH | NGLDGLKGQP | GAPGVKGEPG | APGENGTPGQ | TGARGLPGER | GRVGAPGPAG |
| 2050 | 2060 | 2070 | 2080 | 2090 | 2100 |
| ARGSDGSVGP | VGPAGPIGSA | GPPGFPGAPG | PKGEIGAVGN | AGPAGPAGPR | GEVGLPGLSG |
| 2110 | 2120 | 2130 | 2140 | 2150 | 2160 |
| PVGPPGNPGA | NGLTGAKGAA | GLPGVAGAPG | LPGPRGIPGP | VGAAGATGAR | GLVGEPGPAG |
| 2170 | 2180 | 2190 | 2200 | 2210 | 2220 |
| SKGESGNKGE | PGSAGPQGPP | GPSGEEGKRG | PNGEAGSAGP | PGPPGLRGSP | GSRGLPGADG |
| 2230 | 2240 | 2250 | 2260 | 2270 | 2280 |
| RAGVMGPPGS | RGASGPAGVR | GPNGDAGRPG | EPGLMGPRGL | PGSPGNIGPA | GKEGPVGLPG |
| 2290 | 2300 | 2310 | 2320 | 2330 | 2340 |
| IDGRPGPIGP | AGARGEPGNI | GFPGPKGPTG | DPGKNGDKGH | AGLAGARGAP | GPDGNNGAQG |
| 2350 | 2360 | 2370 | 2380 | 2390 | 2400 |
| PPGPQGVQGG | KGEQGPPGPP | GFQGLPGPSG | PAGEVGKPGE | RGLHGEFGLP | GPAGPRGERG |
| 2410 | 2420 | 2430 | 2440 | 2450 | 2460 |
| PPGESGAAGP | TGPIGSRGPS | GPPGPDGNKG | EPGVVGAVGT | AGPSGPSGLP | GERGAAGIPG |
| 2470 | 2480 | 2490 | 2500 | 2510 | 2520 |
| GKGEKGEPGL | RGEIGNPGRD | GARGAPGAVG | APGPAGATGD | RGEAGAAGPA | GPAGPRGSPG |
| 2530 | 2540 | 2550 | 2560 | 2570 | 2580 |
| ERGEVGPAGP | NGFAGPAGAA | GQPGAKGERG | AKGPKGENGV | VGPTGPVGAA | GPAGPNGPPG |
| 2590 | 2600 | 2610 | 2620 | 2630 | 2640 |
| PAGSRGDGGP | PGMTGFPGAA | GRTGPPGPSG | ISGPPGPPGP | AGKEGLRGPR | GDQGPVGRTG |
| 2650 | 2660 | 2670 | 2680 | 2690 | 2700 |
| EVGAVGPPGF | AGEKGPSGEA | GTAGPPGTPG | PQGLLGAPGI | LGLPGSRGER | GLPGVAGAVG |
| 2710 | 2720 | 2730 | 2740 | 2750 | 2760 |
| EPGPLGIAGP | PGARGPPGAV | GSPGVNGAPG | EAGRDGNPGN | DGPPGRDGQP | GHKGERGYPG |
| 2770 | 2780 | 2790 | 2800 | 2810 | 2820 |
| NIGPVGAAGA | PGPHGPVGPA | GKHGNRGETG | PSGPVGPAGA | VGPRGPSGPQ | GIRGDKGEPG |
| 2830 | 2840 | 2850 | 2860 | 2870 | 2880 |
| EKGPRGLPGL | KGHNGLQGLP | GIAGHHGDQG | APGSVGPAGP | RGPAGPSGPA | GKDGRTGHPG |
| 2890 | 2900 | 2910 | 2920 | | |
| TVGPAGIRGP | QGHQGPAGPP | GPPGPPGPPG | VSGGGYDEGY | DGDFYRA | |

```
SEQ NO 16: Nucleotide Sequence of SPIDCOL1, Codon Optimized for
Nicotiana Benthamiana Chloroplast Expression
      1 ATGGCTACCA CTCTTATATC TAAGTTGACT CTTTCATCAG CTTTCCTTGG ACAACAGTTT
    61 TCATCTAGAG GTAATTCAAT GAGATCAGCA CCAGCTGGAC TTTTTCTTAG GGGACCAAGA
  121 CAAGGAGCAG GTGCTGCTGC AGCAGCAGCT GGAGGTGCTG GACAAGGTGG TTATGGAGGA
  181 CTTGGAGGAC AAGGAGCAGG ACAGGGTGGA TATGGTGGAT TGGGAGGACA AGGTGCTGGA
  241 CAAGGAGCTG GAGCTGCTGC TGCTGCAGCA GCTGGAGGTG CTGGTCAGGG TGGATATGGA
  301 GGATTGGGAA GCCAAGGAGC TGGTAGGGGT GGTCAAGGAG CTGGTGCTGC TGCTGCAGCA
  361 GCTGGTGGAG CAGGTCAGGG CGGTTATGGA GGCTTGGGTT CTCAAGGAGC TGGAAGGGGT
  421 GGCTTGGGTG GCCAAGGTGC CGGTGCTGCT GCTGCTGCTG CTGCTGGTGG TGCTGGTCAA
  481 GGCGGATATG GAGGACTTGG AAACCAAGGT GCTGGCCGTG GTGGACAGGG AGCTGCTGCT
  541 GCTGCTGCAG GAGGAGCTGG TCAGGGTGGG TATGGTGGTT TGGGTTCACA GGGAGCTGGA
  601 AGGGGTGGAC TTGGAGGACA GGGTGCAGGA GCAGCTGCTG CTGCAGCTGG TGGTGCAGGT
  661 CAAGGTGGAT ACGGTGGTCT TGGTGGACAA GGAGCTGGTC AGGGTGGCTA CGGTGGACTT
  721 GGAAGTCAAG GAGCTGGAAG AGGTGGTCTT GGAGGTCAAG GAGCCGGTGC TGCTGCTGCA
  781 GCTGCAGCTG GTGGAGCTGG ACAAGGCGGT CTGGGTGGCC AAGGTGCTGG ACAGGGAGCA
  841 GGTGCATCTG CAGCTGCAGC TGGTGGAGCT GGTCAAGGTG GCTATGGTGG ATTGGGTTCT
  901 CAGGGAGCTG GTAGAGGTGG AGAAGGAGCT GGAGCTGCTG CAGCTGCTGC TGGAGGAGCA
  961 GGTCAGGGTG GTTACGGAGG TTTAGGAGGT CAAGGAGCCG GACAAGGAGG ATATGGAGGT
1021 CTTGGTTCTC AAGGGGCAGG GAGAGGAGGT TTAGGTGGAC AGGGAGCTGG TGCTGCAGCT
1081 GCTGGAGGAG CTGGTCAGGG AGGACTTGGA GGACAAGGTG CAGGTCAAGG TGCTGGTGCA
1141 GCTGCTGCTG CCGCTGGAGG TGCTGGACAG GGAGGGTATG GAGGCCTTGG TAGCCAGGGT
1201 GCAGGCAGGG GAGGTTTGGG AGGACAGGGT GCTGGTGCTG TGGCAGCAGC TGCCGCAGGA
1261 GGTGCTGGAC AAGGAGGATA TGGAGGACTT GGATCTCAAG GTGCTGGTAG AGGTGGTCAA
1321 GGAGCTGGAG CTGCTGCTGC TGCAGCTGGA GGAGCCGGTC AAAGAGGATA CGGTGGACTA
1381 GGTAATCAAG GAGCTGGAAG GGGAGGATTG GGTGGTCAGG GAGCTGGAGC AGCAGCTGCA
1441 GCAGCTGCTG GAGGAGCAGG TCAGGGGGGT TATGGAGGAT TGGGGAATCA AGGTGCAGGA
1501 AGAGGTGGAC AAGGGGCTGC TGCAGCTGCT GGTGGAGCTG GCCAAGGAGG TTACGGTGGA
1561 CTTGGTTCTC AGGGAGCAGG AAGAGGAGGG CAGGGAGCTG GAGCTGCAGC TGCTGCTGCT
1621 GTTGGTGCTG GTCAGGAAGG TATTAGAGGA CAGGGAGCTG GTCAAGGAGG TTACGGAGGT
1681 TTAGGGTCCC AGGGTTCTGG AAGAGGAGGA CTGGGAGGAC AAGGAGCAGG TGCTGCTGCT
1741 GCTGCAGCTG GTGGTGCTGG ACAAGGAGGT CTTGGAGGAC AAGGAGCTGG ACAGGGAGCT
1801 GGTGCAGCTG CTGCTGCTGC TGGAGGAGTT AGACAGGGAG GATATGGAGG TTTGGGATCA
1861 CAAGGTGCAG GAAGAGGTGG ACAGGGAGCT GGAGCTGCAG CTGCTGCGGC TGGTGGGGCT
1921 GGACAAGGTG GATATGGAGG GCTTGGAGGC CAAGGAGTTG GAAGGGGTGG GCTTGGTGGA
1981 CAAGGTGCAG GTGCTGCTGCT GCTGGAGGT GCTGGTCAAG GCGGTTACGG AGGTGTTGGT
2041 TCTGGAGCTT CAGCTGCAAG TGCTGCAGCT AGTAGGCTTT CTAGTCCACA AGCATCATCT
2101 AGAGTTTCTT CTGCTGTTTC TAATTTGGTG GCATCTGGTC CAACAAACTC GGCAGCACTT
2161 TCTTCTACTA TTTCTAATGT TGTTTCTCAG ATAGGTGCAT CTAACCCAGG TCTTTCAGGA
2221 TGTGATGTTT TGATACAGGC TTTGCTTGAA GTGGTTAGTG CTCTTATACA AATTCTCGGA
2281 TCCTCATCAA TTGGTCAAGT GAACTACGGT TCTGCTGGAC AAGCTACACA GATTGTTGGT
2341 CAATCAGTTT ATCAAGCACT TGGGGGTTCT GGTGAAGGAA GGGGTAGTCT TCTTACTTGT
2401 GAAGATGTGG AAGAAAATCC TGGACCACAG CTTTCTTATG GATACGATGA AAAGTCTACT
2461 GGAGGTATAT CTGTTCCTGG TCCTATGGGA CCTAGTGGTC CTAGAGGTTT GCCAGGACCT
2521 CCTGGTGCTC CAGGACCACA AGGATTTCAG GGACCACCAG GGGAACCAGG TGAACCTGGA
2581 GCTTCTGGAC CAATGGGTCC TAGAGGTCCA CCTGGACCTC CTGGTAAAAA TGGAGATGAT
2641 GGTGAGGCTG GAAAGCCAGG AAGGCCAGGA GAAAGAGGTC CACCAGGACC ACAGGGTGCT
2701 CGTGGTCTTC CAGGAACAGC CGGTTTACCT GGCATGAAGG GACATAGAGG ATTTTCAGGT
2761 TTGGATGGAG CTAAAGGAGA TGCTGGACCA GCTGGACCTA AAGGAGAGCC AGGATCTCCT
2821 GGAGAGAATG GTGCACCTGG CCAGATGGGT CCAAGGGGTC TTCCAGGTGA GAGAGGTAGA
2881 CCTGGAGCCC CAGGTCCAGC AGGCGCTAGA GGGAATGACG GAGCCACAGG TGCAGCTGGT
2941 CCACCTGGAC CTACTGGTCC TGCTGGGCCT CCTGGCTTTC CTGGAGCTGT AGGTGCTAAG
3001 GGTGAGGCTG GACCTCAAGG TCCTCGAGGA TCAGAAGGTC CACAAGGAGT TAGGGGAGAG
3061 CCTGGCCCAC CAGGTCCAGC TGGAGCTGCA GGTCCTGCTG GTAATCCAGG AGCTGATGGA
3121 CAACCTGGAG CTAAAGGTGC TAACGGAGCT CCTGGAATTG CTGGAGCACC AGGTTTTCCT
3181 GGTGCTAGAG GACCATCAGG ACCACAAGGA CCAGGTGGTC CTCCAGGACC TAAAGGTAAT
3241 AGTGGAGAAC CAGGTGCTCC TGGTTCTAAA GGAGATACTG GTGCTAAGGG AGAACCAGGC
3301 CCTGTTGGAG TCCAAGGTCC ACCTGGACCA GCTGGAGAAG AAGGAAAGAG GGGAGCTAGA
3361 GGCGAACCAG GACCTACTGG ATTGCCAGGT CCTCCAGGTG AAAGAGGAGG TCCAGGTTCT
3421 AGGGGTTTCC CAGGAGCAGA TGGAGTAGCT GGACCTAAGG GACCCGCTGG TGAAAGAGGT
3481 TCACCTGGAC CTGCAGGTCC TAAGGGTTCA CCAGGTGAAG CAGGTAGACC TGGTGAAGCA
3541 GGTTTGCCTG GAGCTAAGGG TTTGACAGGA AGTCCAGGGT CACCTGGACC AGATGGAAAG
3601 ACAGGACCTC CTGGTCCAGC TGGTCAAGAT GGAAGACCTG GTCCTCCAGG ACCACCAGGT
3661 GCAAGAGGAC AAGCTGGAGT TATGGGTTTT CCTGGTCCAA AGGGAGCTGC TGGAGAGCCA
3721 GGTAAAGCTG GTGAAAGAGG TGTTCCAGGT CCTCCTGGTG CTGTTGGACC AGCTGGTAAA
3781 GATGGAGAAG CTGGAGCTCA AGGTCCACCT GGTCCTGCAG GACCAGCTGG AGAAAGAGGC
3841 GAACAAGGTC CTGCTGGTTC GCCAGGATTT CAGGGTTTAC CAGGTCCCGC TGGTCCTCCA
3901 GGTGAAGCTG GAAAACCTGG AGAACAAGGT GTGCCTGGAG ATTTGGGAGC TCCAGGACCT
3961 TCTGGTGCAA GAGGTGAGCG TGGTTTCCCT GGAGAAAGGG GTGTTCAAGG TCCACCTGGA
4021 CCTGCTGGTC CTAGAGGAGC TAACGGAGCT CCAGGAAATG ATGGTGCAAA GGGTGATGCT
4081 GGTGCACCTG GTGCTCCTGG ATCTCAAGGT GCTCCAGGTC TTCAGGGTAT GCCAGGAGAG
4141 AGGGGAGCTG CTGGATTACC TGGGCCTAAA GGTGATAGAG GAGATGCTGG TCCAAAGGGT
4201 GCTGATGGTA GTCCAGGTAA AGATGGTGTT AGAGGACTTA CAGGCCCTAT TGGTCCACCT
4261 GGGCCAGCTG GTGCACCAGG TGATAAGGGA GAAAGTGGAC CAAGTGGACC AGCAGGACCA
4321 ACCGGTGCTA GAGGAGCACC AGGTGATAGA GGAGAACCAG GTCCACCAGG ACCAGCTGGT
4381 TTTGCTGGTC CTCCAGGAGC TGATGGACAA CCAGGAGCTA AAGGTGAGCC TGGAGATGCT
4441 GGAGCTAAAG GAGATGCTGG TCCACCGGGA CCAGCAGGTC CAGCAGGCCC ACCAGGTCCA
4501 ATTGGAAACG TTGGTGCACC TGGCGCTAAG GGTGCCAGAG GAAGCGCAGG TCCACCAGGA
4561 GCAACTGGCT TTCCAGGTGC TGCAGGTAGA GTTGGACCAC CAGGACCTTC TGGAAACGCT
4621 GGACCTCCTG GGCCTCCAGG ACCTGCTGGA AAGGAAGGAG GGAAGGGTCC TAGGGGAGAG
4681 ACTGGACCAG CTGGTAGACC AGGTGAGGTT GGACCACCAG GTCCTCCAGG CCCAGCTGGT
4741 GAAAAGGGTA GTCCCGGTGC TGATGGACCA GCAGGAGCTC CAGGAACACC AGGACCTCAA
4801 GGTATTGCTG GTCAAAGAGG TGTTGTTGGT TTGCCTGGTC AGAGAGGAGA AAGAGGATTT
4861 CCAGGATTGC CAGGACCTTC TGGTGAGCCT GGTAAACAGG GTCCATCAGG TGCTTCTGGG
4921 GAAAGAGGAC CACCTGGTCC TATGGGACCA CCAGGTTTGG CTGGTCCTCC TGGTGAATCA
4981 GGTAGGGAAG GAGCTCCCGG AGCTGAAGGA TCACCAGGAA GAGATGGATC TCCTGGAGCT
5041 AAAGGAGATA GAGGAGAAAC AGGTCCAGCT GGACCACCAG GAGCACCTGG TGCTCCTGGT
5101 GCTCCTGGAC CTGTTGGTCC AGCTGGTAAA TCAGGAGATA GAGGTGAAAC TGGACCTGCT
5161 GGACCAGCTG GTCCAGTTGG ACCTGTTGGT GCTAGAGGGC CAGCAGGACC ACAGGGTCCT
5221 AGAGGAGATA AGGGAGAGAC TGGTGAACAA GGAGATAGAG GAATCAAAGG TCATAGAGGA
5281 TTTAGTGGAC TTCAAGGACC ACCTGGCCCT CCTGGTTCTC CTGGAGAACA AGGCCCATCT
5341 GGTGCTTCTG GACCTGCTGG CCCAAGGGGA CCACCTGGAT CTGCTGGTGC CCCTGGTAAA
5401 GATGGACTTA ATGGATTGCC AGGTCCAATT GGTCCTCCTG GTCCAAGAGG AAGGACAGGA
5461 GATGCTGGAC CTGTTGGTCC TCCTGGGCCA CCAGGACCAC CTGGACCTCC TGGACCTCCA
5521 AGTGCTGGTT TTGATTTCTC TTTTTTACCA CAGCCACCAC AGGAAAAAGC TCATGATGGT
5581 GGAAGATACT ATAGAGCTGG TTCAGGTGAG GGTAGAGGAT CCTTACTTAC ATGTGAAGAT
5641 GTTGAGGAAA ATCCTGGACC ACAGTACGAT GGAAAAGGTG TTGGACTTGG TCCAGGTCCA
5701 ATGGGATTGA TGGGACCAAG AGGACCTCCA GGAGCTGCAG GAGCTCCAGG ACCACAGGGA
5761 TTTCAAGGTC CTGCAGGAGA GCCTGGAGAG CCAGGTCAAA CTGGACCTGC AGGAGCTAGA
5821 GGTCCAGCTG GACCTCCAGG TAAAGCTGGA GAAGATGGTC ATCCAGGAAA GCCAGGGAGG
5881 CCAGGTGAAA GGGGTGTTGT TGGTCCACAG GGGGCTAGAG GCTTCCCTGG TACACCTGGT
5941 CTTCCAGGAT TTAAAGGTAT TAGAGGTCAT AATGGTTTAG ATGGATTGAA GGGTCAACCA
6001 GGAGCTCCAG GTGTTAAGGG GGAACCAGGA GCACCAGGTG AAAATGGAAC TCCTGGTCAG
6061 ACTGGAGCTA GAGGACTTCC AGGAGAAAGA GGTAGAGTGG GTGCACCTGG TCCAGCAGGG
6121 GCTCGTGGTA GTGATGGTTC CGTTGGACCC GTCGGACCTG CAGGTCCAAT TGGATCAGCA
6181 GGACCACCTG GATTCCCAGG AGCTCCAGGT CCAAAAGGCG AGATTGGTGC TGTTGGAAAT
6241 GCTGGGCCTG CTGGACCTGC TGGTCCTAGA GGAGAGGTTG GACTTCCAGG TTTGTCCGGA
6301 CCAGTGGGAC CACCTGGAAA TCCAGGAGCT AATGGTCTTA CTGGAGCTAA AGGAGCTGCA
6361 GGGTTGCCTG GTGTTGCTGG AGCTCCAGGA CTTCCTGGAC CTAGAGGAAT TCCTGGTCCA
6421 GTTGGAGCTG CTGGTGCTAC TGGTGCTAGA GGACTTGTTG GAGAACCAGG TCCAGCAGGA
6481 TCTAAGGGAG AGTCAGGTAA TAAAGGTGAG CCAGGAAGTG CTGGTCCACA AGGTCCACCA
6541 GGACCTTCTG GTGAGGAGGG TAAGAGGGGT CCAAATGGTG AAGCTGGATC AGCTGGACCT
6601 CCAGGACCAC CTGGACTTAG GGGTAGCCCT GGTTCAAGAG GACTGCCTGG GGCTGATGGA
6661 AGAGCTGGAG TTATGGGACC TCCCGGTAGT AGGGGAGCAT CCGGACCAGC TGGAGTAAGG
6721 GGACCTAATG GTGATGCTGG AAGACCAGGA GAACCTGGAT TAATGGGTCC TAGGGGTCTC
6781 CCAGGATCTC CAGGTAACAT TGGTCCTGCT GGTAAAGAAG GACCAGTTGG TCTTCCAGGC
6841 ATTGATGGTA GACCAGGACC AATTGGGCCA GCTGGTGCTC GTGGCGAACC TGGTAATATA
6901 GGATTCCCAG GTCCTAAGGG ACCAACCGGT GATCCAGGTA AAAATGGTGA TAAAGGTCAT
6961 GCTGGATTGG CCGGAGCTAG GGGAGCTCCA GGTCCAGATG GAAATAATGG TGCTCAGGGA
7021 CCACCAGGAC CACAGGGTGT TCAAGGTGGA AAAGGAGAAC AGGGTCCTCC TGGTCCTCCA
7081 GGTTTCCAAG GACTTCCTGG ACCTTCTGGT CCAGCAGGTG AGGTTGGTAA ACCAGGAGAG
7141 AGAGGATTGC ACGGAGAATT TGGTTTGCCA GGACCGGCTG GTCCTAGGGG TGAAAGAGGA
7201 CCACCTGGTG AATCTGGAGC TGCTGGACCA ACTGGTCCTA TTGGTTCAAG GGGACCTTCT
7261 GGACCTCCAG GTCCAGATGG AAATAAAGGA GAGCCAGGAG TGGTTGGAGC TGTTGGAACA
7321 GCTGGACCAA GTGGACCTTC AGGACTCCCA GGAGAGAGGG GCGCTGCTGG TATTCCTGGT
7381 GGAAAAGGTG AGAAGGGTGA GCCTGGACTT AGAGGAGAAA TAGGAAATCC AGGCAGGGAT
7441 GGTGCACGGG GAGCTCCTGG AGCTGTTGGT GCCCCAGGAC CAGCCGGAGC AACAGGAGAT
7501 AGGGGAGAGG CTGGTGCTGC TGGTCCAGCT GGACCTGCAG GACCTAGGGG TTCACCAGGA
7561 GAAAGAGGTG AGGTTGGTCC AGCTGGTCCT AATGGATTTG CTGGTCCTGC TGGTGCTGCT
7621 GGTCAACCTG GAGCTAAGGG TGAGAGGGGT GCAAAAGGAC CTAAAGGTGA AAATGGTGTT
7681 GTTGGTCCTA CTGGACCAGT TGGAGCTGCT GGACCTGCTG GACCAAATGG TCCACCTGGT
7741 CCAGCTGGTT CTAGAGGAGA TGGTGGGCCA CCTGGAATGA CTGGATTCCC AGGTGCTGCT
7801 GGAAGGACTG GACCACCAGG CCCTAGTGGA ATTTCTGGAC CACCTGGTCC TCCTGGACCA
7861 GCAGGTAAGG AAGGTTTGAG GGGACCAAGA GGGGATCAGG GACCTGTAGG TAGAACTGGT
7921 GAGGTTGGTG CTGTTGGCCC ACCAGGTTTC GCTGGCGAAA AGGGACCTTC AGGTGAAGCT
7981 GGTACAGCTG GTCCTCCTGG TACTCCTGGT CCACAAGGTT TGCTTGGTGC TCCTGGTATT
8041 CTTGGTCTTC CAGGTTCAAG AGGTGAGAGA GGTCTTCCTG GAGTGGCTGG AGCTGTTGGA
8101 GAACCAGGTC CATTGGGTAT AGCTGGACCT CCAGGCGCTA GAGGCCCACC TGGTGCAGTC
8161 GGATCACCAG GTGTTAACGG AGCTCCAGGT GAGGCAGGTA GAGATGGAAA TCCTGGAAAT
8221 GATGGGCCTC CTGGTAGGGA TGGACAGCCA GGTCATAAAG GTGAAAGAGG ATACCCTGGA
8281 AATATCGGTC CTGTTGGTGC TGCTGGTGCA CCAGGACCAC ATGGTCCTGT TGGTCCTGCT
8341 GGAAAGCATG GTAATCGAGG AGAAACTGGA CCATCTGGAC CAGTTGGTCC AGCAGGTGCT
8401 GTTGGACCAC GAGGACCTTC AGGACCACAG GGAATTAGGG GTGATAAGGG CGAGCCTGGT
8461 GAAAAGGGAC CTAGGGGTCT TCCAGGTTTG AAAGGTCATA ACGGACTGCA AGGACTTCCA
8521 GGAATTGCTG GTCACCACGG TGATCAAGGA GCCCCAGGTT CTGTTGGTCC AGCTGGACCA
8581 AGAGGACCAG CAGGTCCATC AGGTCCAGCT GGAAAAGATG GTAGAACTGG ACATCCAGGC
8641 ACAGTTGGTC CAGCTGGTAT TAGGGGACCT CAAGGTCATC AAGGACCAGC TGGACCTCCA
8701 GGACCACCTG GTCCACCAGG ACCACCAGGT GTTTCTGGAG GAGGCTACGA TTTTGGTTAT
8761 GATGGTGATT TTTATAGGGC TTAA

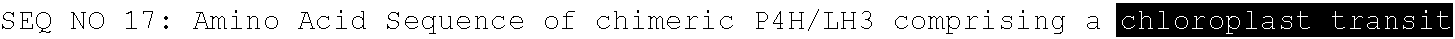

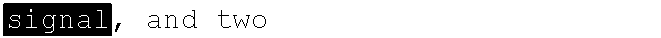

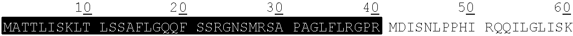



```

| | | | | | |
|---|---|---|---|---|---|
| 70 | 80 | 90 | 100 | 110 | 120 |
| PQQNNDESSS | SNNKNNLINN | EKVSNVLIDL | TSNLKIENFK | IFNKESLNQL | EKKGYLIIDN |
| 130 | 140 | 150 | 160 | 170 | 180 |
| FLNDLNKINL | IYDESYNQFK | ENKLIEAGMN | KGTDKWKDKS | IRGDYIQWIH | RDSNSRIQDK |
| 190 | 200 | 210 | 220 | 230 | 240 |
| DLSSTIRNIN | YLLDKLDLIK | NEFDNVIPNF | NSIKTQTQLA | VYLNGGRYIK | HRDSFYSSES |
| 250 | 260 | 270 | 280 | 290 | 300 |
| LTISRRITMI | YYVNKDWKKG | DGGELRLYTN | NPNNTNQKEL | KQTEEFIDIE | PIADRLLIFL |

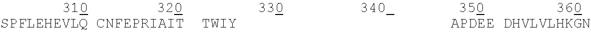

| | | | | | |
|---|---|---|---|---|---|
| 370 | 380 | 390 | 400 | 410 | 420 |
| FDEALAAHKY | LLVEFYAPWC | GHCKALAPEY | AKAAGKLKAE | GSEIRLAKVD | ATEESDLAQQ |
| 430 | 440 | 450 | 460 | 470 | 480 |
| YGVRGYPTIK | FFKNGDTASP | KEYTAGREAD | DIVNWLKKRT | GPAASTLSDG | AAAEALVESS |
| 490 | 500 | 510 | 520 | 530 | 540 |
| EVAVIGFFKD | MESDSAKQFF | LAAEVIDDIP | FGITSNSDVF | SKYQLDKDGV | VLFKKEDEGR |
| 550 | 560 | 570 | 580 | 590 | 600 |
| NNFEGEVTKE | KLLDFIKHNQ | LPLVIEFTEQ | TAPKIFGGEI | KTHILLFLPK | SVSDYEGKLS |
| 610 | 620 | 630 | 640 | 650 | 660 |
| NEKKAAESFK | GKILFIFIDS | DHTDNQRILE | FFGLKKEECP | AVRLITLEEE | MTKYKPESDE |
| 670 | 680 | 690 | 700 | 710 | 720 |
| LTAEKITEFC | HRFLEGKIKP | HLMSQELPDD | WDKQPVKVLV | GKNFEEVAFD | EKKNVEVEFY |
| 730 | 740 | 750 | 760 | 770 | 780 |
| APWCGHCKQL | APIWDKLGET | YKDHENIVIA | KMDSTANEVE | AVKVHSFPTL | KFFPASADRT |

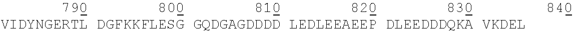

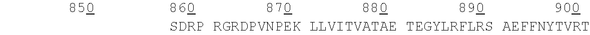

| | | | | | |
|---|---|---|---|---|---|
| 910 | 920 | 930 | 940 | 950 | 960 |
| LGLGEEWRGG | DVARTVGGGQ | KVRWLKKEME | KYADREDMII | MFVDSYDVIL | AGSPTELLKK |
| 970 | 980 | 990 | 1000 | 1010 | 1020 |
| FVQSGSRLLF | SAESFCWPEW | GLAEQYPEVG | TGKRFLNSGG | FIGFATTIHQ | IVRQWKYKDD |
| 1030 | 1040 | 1050 | 1060 | 1070 | 1080 |
| DDDQLFYTRL | YLDPGLREKL | SLNLDHKSRI | FQNLNGALDE | VVLKFDRNRV | RIRNVAYDTL |
| 1090 | 1100 | 1110 | 1120 | 1130 | 1140 |
| PIVVHGNGPT | KLQLNYLGNY | VPNGWTPEGG | CGFCNQDRRT | LPGGQPPPRV | FLAVFVEQPT |
| 1150 | 1160 | 1170 | 1180 | 1190 | 1200 |
| PFLPRFLQRL | LLLDYPPDRV | TLFLHNNEVF | HEPHIADSWP | QLQDHFSAVK | LVGPEEALSP |
| 1210 | 1220 | 1230 | 1240 | 1250 | 1260 |
| GEARDMAMDL | CRQDPECEFY | FSLDADAVLT | NLQTLRILIE | ENRKVIAPML | SRHGKLWSNF |
| 1270 | 1280 | 1290 | 1300 | 1310 | 1320 |
| WGALSPDEYY | ARSEDYVELV | QRKRVGVWNV | PYISQAYVIR | GDTLRMELPQ | RDVFSGSDTD |
| 1330 | 1340 | 1350 | 1360 | 1370 | 1380 |
| PDMAFCKSFR | DKGIFLHLSN | QHEFGRLLAT | SRYDTEHLHP | DLWQIFDNPV | DWKEQYIHEN |
| 1390 | 1400 | 1410 | 1420 | 1430 | 1440 |
| YSRALEGEGI | VEQPCPDVYW | FPLLSEQMCD | ELVAEMEHYG | QWSGGRHEDS | RLAGGYENVP |
| 1450 | 1460 | 1470 | 1480 | 1490 | 1500 |
| TVDIHMKQVG | YEDQWLQLLR | TYVGPMTESL | FPGYHTKARA | VMNFVVRYRP | DEQPSLRPHH |
| 1510 | 1520 | 1530 | 1540 | 1550 | 1560 |
| DSSTFTLNVA | LNHKGLDYEG | GGCRFLRYDC | VISSPRKGWA | LLHPGRLTHY | HEGLPTTWGT |
| 1570 | | | | | |
| RYIMVSFVDP | | | | | |

```
SEQ NO 18: Nucleotide Sequence of chimeric P4H/LH3, Codon Optimized
for Nicotiana Benthamiana Chloroplast Expression
      1 ATGGCAACAA CACTTATTAG TAAACTCACT CTTTCTAGTG CTTTTCTTGG ACAGCAATTT
    61 TCTAGCAGGG GAAATTCTAT GAGAAGTGCT CCAGCCGGTT TATTTTTGCG CGGTCCTAGA
  121 ATGGATATAA GTAACTTGCC ACCACATATT AGACAGCAAA TTCTTGGTCT TATCTCAAAG
  181 CCTCAACAGA ACAATGATGA ATCTTCATCA TCTAATAATA AGAATAATCT TATCAATAAC
  241 GAAAAGGTTT CTAATGTTCT TATTGATCTT ACTTCTAATT TGAAGATTGA AAATTTTAAA
  301 ATTTTTAATA AAGAGTCACT TAATCAACTC GAAAAAAAGG GATACCTCAT AATTGATAAT
  361 TTCTTAAATG ACCTTAATAA GATTAATCTT ATTTATGATG AATCTTATAA CCAATTTAAG
  421 GAAAACAAGC TTATTGAAGC TGGTATGAAT AAGGGTACAG ATAAATGGAA AGATAAGAGT
  481 ATTAGAGGGG ATTATATTCA GTGGATTCAT AGAGATTCCA ATTCTAGAAT TCAAGATAAG
  541 GATCTTTCAA GTACAATTAG AAATATTAAT TATTTGTTGG ACAAGTTGGA TCTTATTAAG
  601 AATGAGTTTG ATAACGTTAT CCCTAATTTT AATTCTATCA AGACTCAAAC CCAATTGGCT
  661 GTATATTTGA ACGGAGGAAG ATACATTAAA CATAGGGATA GTTTTTATTC CTCAGAATCT
  721 TTGACTATTA GCAGAAGAAT TACTATGATT TATTATGTCA ATAAAGACTG GAAAAAGGGA
  781 GATGGAGGAG AGCTTAGACT GTACACTAAT AACCCAAACA ATACTAATCA AAAAGAGTTG
  841 AAACAAACTG AAGAATTTAT TGATATAGAA CCAATAGCAG ACAGATTGCT TATTTTTTTG
  901 TCTCCATTTC TTGAACATGA GGTTCTTCAA TGTAATTTTG AACCACGTAT TGCTATTACT
  961 ACATGGATTT ATGGATCTGG CGAGGGTAGG GGTTCACTCC TTACTTGTGA GGATGTTGAA
1021 GAGAATCCTG GACCAGCACC AGATGAGGAA GATCATGTTT TGGTTCTTCA TAAAGGAAAT
1081 TTTGATGAAG CTTTGGCTGC TCACAAATAT TTGCTTGTTG AATTCTATGC TCCTTGGTGT
1141 GGTCATTGCA AGGCATTGGC CCCTGAGTAT GCTAAGGCAG CTGGAAAGTT GAAGGCCGAG
1201 GGATCTGAAA TTAGACTTGC AAAGGTTGAC GCTACTGAGG AATCTGATTT GGCACAACAA
1261 TATGGTGTTA GAGGTTACCC AACTATTAAG TTCTTTAAGA ATGGTGACAC TGCTAGTCCT
1321 AAGGAATATA CCGCTGGTAG AGAGGCCGAT GATATCGTAA ATTGGCTTAA GAAAAGGACA
1381 GGACCAGCAG CTTCAACATT GTCAGATGGT GCTGCTGCTG AAGCTTTAGT CGAATCTTCA
1441 GAAGTTGCTG TGATTGGATT TTTTAAAGAC ATGGAATCTG ATAGTGCCAA ACAGTTTTTT
1501 TTGGCTGCAG AGGTGATTGA TGATATTCCA TTTGGAATTA CTTCAAATTC AGATGTGTTT
1561 TCAAAATATC AGCTTGATAA GGACGGAGTT GTTTTGTTCA AAAAGTTCGA TGAAGGAAGA
1621 AATAATTTTG AAGGTGAAGT GACTAAAGAA AAGCTTCTTG ATTTTATTAA GCACAATCAA
1681 TTGCCATTGG TTATTGAATT TACTGAGCAA ACTGCTCCAA AGATTTTTGG TGGTGAAATT
1741 AAGACTCATA TTCTTTTGTT CTTGCCTAAG TCTGTTAGTG ATTATGAAGG TAAGTTGAGC
1801 AACTTTAAAA AGGCTGCTGA ATCTTTTAAG GGAAAAATTC TTTTTATCTT CATTGATAGC
1861 GATCACACAG ATAATCAGAG AATATTGGAA TTCTTCGGTT TGAAGAAGGA AGAATGCCCT
1921 GCTGTTAGGT TGATTACACT GGAGGAGGAG ATGACTAAGT ACAAGCCTGA ATCTGATGAG
1981 CTTACTGCTG AAAAGATCAC TGAATTCTGC CACAGATTTC TTGAGGGGAA GATTAAGCCA
2041 CACCTTATGT CTCAGGAGTT ACCTGATGAT TGGGATAAAC AACCTGTTAA GGTTCTCGTG
2101 GGTAAGAACT TTGAAGAGGT TGCTTTCGAT GAAAAAAAAA ATGTTTTCGT TGAATTCTAT
2161 GCACCTTGGT GCGGTCATTG TAAACAGCTA GCACCAATTT GGGATAAACT TGGGGAAACT
2221 TATAAGGATC ATGAGAATAT TGTTATAGCT AAAATGGATT CAACGGCTAA TGAAGTTGAA
2281 GCTGTTAAAG TCCATTCATT TCCTACTCTT AAGTTCTTTC CTGCTTCAGC AGACCGTACC
2341 GTTATTGATT ACAATGGTGA GAGAACATTG GATGGCTTTA AAAAATTTTT GGAATCTGGC
2401 GGACAGGATG GAGCTGGAGA TGATGATGAC TTGGAGGATT TAGAGGAGGC CGAAGAGCCT
2461 GATTTGGAAG AAGATGATGA TCAAAAAGCA GTGAAGGATG AACTAGGTTC AGGAGAGGGT
2521 AGGGGGAGTT TGTTGACTTG CGAGGATGTA GAAGAAAACC CTGGTCCATC TGATAGACCT
2581 AGAGGTAGAG ATCCTGTTAA CCCAGAGAAG CTTTTGGTTA TTACAGTGGC TACAGCTGAG
2641 ACAGAAGGAT ATCTTAGGTT TCTAAGGTCT GCTGAATTTT TTAATTATAC AGTTAGAACA
2701 TTGGGACTTG GGGAGGAATG GAGAGGTGGG GATGTTGCTC GAACTGTGGG AGGAGGTCAA
2761 AAGGTTCGTT GGTTGAAGAA AGAAATGGAA AAATATGCAG ATAGAGAAGA TATGATTATT
2821 ATGTTTGTTG ATAGTTACGA TGTTATTTTG GCTGGAAGCC CTACAGAATT GTTAAAGAAG
2881 TTTGTTCAAT CTGGCTCAAG GCTTTTGTTC TCCGCAGAGA GTTTCTGCTG GCCAGAGTGG
2941 GGACTAGCTG AACAGTATCC TGAGGTGGGT ACTGGTAAGA GGTTTCTCAA TTCCGGTGGT
3001 TTTATTGGCT TCGCAACTAC TATTCACCAA ATTGTTAGAC AATGGAAGTA TAAAGATGAT
3061 GATGATGATC AACTTTTTTA TACAAGACTT TACCTTGACC CAGGTTTGAG AGAAAAGTTG
3121 TCTCTGAACT TGGATCACAA GTCTAGAATT TTCCAAAATC TCAATGGAGC TTTGGATGAA
3181 GTTGTTTTGA AATTTGATAG AAATAGGGTT AGGATTCGTA ATGTTGCCTA TGACACACTT
3241 CCTATTGTAG TGCATGGAAA TGGACCTACT AAGCTTCAGT TGAACTATTT AGGTAACTAT
3301 GTGCCTAACG GATGGACTCC AGAAGGTGGT TGTGGATTTT GTAATCAAGA TCGAAGAACT
3361 TTGCCAGGAG GACAACCTCC ACCAAGGGTT TTTCTTGCTG TTTTCGTTGA GCAACCTACC
3421 CCATTCCTTC CAAGATTCTT ACAAAGACTT TTGTTGCTTG ATTATCCACC AGATAGAGTT
3481 ACTTTGTTCC TTCACAATAA TGAGGTGTTT CATGAGCCTC ATATTGCTGA TAGTTGGCCA
3541 CAACTCCAAG ATCATTTCTC CGCAGTTAAG CTCGTTGGTC CAGAGGAAGC TTTGTCTCCT
3601 GGTGAAGCTA GGGATATGGC AATGGATCTT TGCAGACAAG ATCCTGAATG CGAATTTTAC
3661 TTTTCTTTGG ATGCTGATGC TGTGCTTACA AATCTTCAGA CTCTAAGAAT TTTGATAGAG
3721 GAGAACAGGA AAGTTATTGC TCCAATGCTT AGTAGGCATG GTAAATTGTG GAGTAATTTC
3781 TGGGGAGCTC TTTCTCCGGA TGAATATTAT GCTAGATCGG AAGATTACGT GGAGCTTGTT
3841 CAACGTAAGA GAGTTGGTGT ATGGAATGTT CCATATATCT CACAAGCTTA CGTTATCAGA
3901 GGAGATACAT TGAGAATGGA ACTTCCTCAG AGAGATGTTT TTAGCGGATC AGATACCGAT
3961 CCTGATATGG CATTTTGTAA ATCATTCAGA GATAAGGGAA TTTTCCTTCA TCTATCTAAT
4021 CAGCACGAAT TCGGAAGGTT GCTTGCTACA TCAAGATATG ATACTGAGCA CCTGCATCCA
4081 GATTTGTGGC AAATTTTCGA TAATCCAGTG GATTGGAAAG AACAATACAT ACATGAAAAT
4141 TATTCTAGAG CTCTTGAAGG TGAGGGAATT GTCGAACAAC CTTGCCCAGA CGTCTATTGG
4201 TTTCCACTTC TTTCTGAGCA AATGTGCGAT GAACTAGTGG CAGAGATGGA ACATTACGGA
4261 CAATGGTCTG GAGGACGGCA TGAGGATTCA AGATTGGCTG GAGGGTACGA GAATGTGCCA
4321 ACTGTCGATA TTCATATGAA GCAAGTTGGA TATGAAGATC AGTGGTTGCA ACTTTTAAGA
4381 ACATACGTTG GTCCTATGAC TGAATCATTG TTTCCAGGAT ACCACACAAA AGCAAGAGCA
4441 GTTATGAATT TCGTTGTTAG ATACAGACCA GATGAGCAAC CTTCTTTAAG ACCACATCAT
4501 GATTCTTCTA CATTTACTCT CAATGTTGCT TTGAATCACA AAGGTCTTGA TTATGAGGGA
4561 GGAGGATGCA GGTTTCTGAG ATATGATTGT GTAATTTCAT CGCCTCGTAA AGGATGGGCT
4621 TTGCTCCATC CAGGAAGACT TACTCACTAT CATGAAGGAC TCCCTACTAC ATGGGGTACT
4681 AGATATATTA TGGTTTCATT TGTTGATCCT TGA

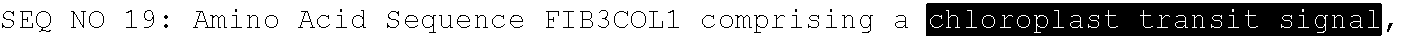

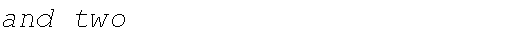

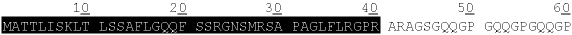



```

| | | | | | |
|---|---|---|---|---|---|
| 70 | 80 | 90 | 100 | 110 | 120 |
| GQQGPYGPGA | SAAAAAAGGY | GPGSGQQGPS | QQGPGQQGPG | GQGPYGPGAS | AAAAAAGGYG |
| 130 | 140 | 150 | 160 | 170 | 180 |
| PGSGQQGPGG | QGPYGPGSSA | AAAAAGGNGP | GSGQQGAGQQ | GPGQQGPGAS | AAAAAAGGYG |
| 190 | 200 | 210 | 220 | 230 | 240 |
| PGSGQQGPGQ | QGPGGQGPYG | PGASAAAAAA | GGYGPGSGQG | PGQQGPGGQG | PYGPGASAAA |
| 250 | 260 | 270 | 280 | 290 | 300 |
| AAAGGYGPGS | GQQGPGQQGP | GQQGPGGQGP | YGPGASAAAA | AAGGYGPGYG | QQGPGQQGPG |
| 310 | 320 | 330 | 340 | 350 | 360 |
| GQGPYGPGAS | AASAASGGYG | PGSGQQGPGQ | QGPGGQGPYG | PGASAAAAAA | GGYGPGSGQQ |
| 370 | 380 | 390 | 400 | 410 | 420 |
| GPGQQGPGQQ | GPGQQGPGGQ | GPYGPGASAA | AAAAGGYGPG | SGQQGPGQQG | PGQQGPGQQG |
| 430 | 440 | 450 | 460 | 470 | 480 |
| PGQQGPGQQG | PGQQGPGQQG | PGQQGPGGQG | AYGPGASAAA | GAAGGYGPGS | GQQGPGQQGP |
| 490 | 500 | 510 | 520 | 530 | 540 |
| GQQGPGQQGP | GQQGPGQQGP | GQQGPGQQGP | YGPGASAAAA | AAGGYGPGSG | QQGPGQQGPG |
| 550 | 560 | 570 | 580 | 590 | 600 |
| QQGPGGQGPY | GPGAASAAVS | VGGYGPQSSS | VPVASAVASR | LSSPAASSRV | SSAVSSLVSS |
| 610 | 620 | 630 | 640 | 650 | 660 |
| GPTKHAALSN | TISSVVSQVS | ASNPGLSGCD | VLVQALLEVV | SALVSILGSS | SIGQINYGAS |

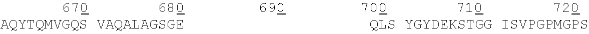

| | | | | | |
|---|---|---|---|---|---|
| 730 | 740 | 750 | 760 | 770 | 780 |
| GPRGLPGPPG | APGPQGFQGP | PGEPGEPGAS | GPMGPRGPPG | PPGKNGDDGE | AGKPGRPGER |
| 790 | 800 | 810 | 820 | 830 | 840 |
| GPPGPQGARG | LPGTAGLPGM | KGHRGFSGLD | GAKGDAGPAG | PKGEPGSPGE | NGAPGQMGPR |
| 850 | 860 | 870 | 880 | 890 | 900 |
| GLPGERGRPG | APGPAGARGN | DGATGAAGPP | GPTGPAGPPG | FPGAVGAKGE | AGPQGPRGSE |
| 910 | 920 | 930 | 940 | 950 | 960 |
| GPQGVRGEPG | PPGPAGAAGP | AGNPGADGQP | GAKGANGAPG | IAGAPGFPGA | RGPSGPQGPG |
| 970 | 980 | 990 | 1000 | 1010 | 1020 |
| GPPGPKGNSG | EPGAPGSKGD | TGAKGEPGPV | GVQGPPGPAG | EEGKRGARGE | PGPTGLPGPP |
| 1030 | 1040 | 1050 | 1060 | 1070 | 1080 |
| GERGGPGSRG | FPGADGVAGP | KGPAGERGSP | GPAGPKGSPG | EAGRPGEAGL | PGAKGLTGSP |
| 1090 | 1100 | 1110 | 1120 | 1130 | 1140 |
| GSPGPDGKTG | PPGPAGQDGR | PGPPGPPGAR | GQAGVMGFPG | PKGAAGEPGK | AGERGVPGPP |
| 1150 | 1160 | 1170 | 1180 | 1190 | 1200 |
| GAVGPAGKDG | EAGAQGPPGP | AGPAGERGEQ | GPAGSPGFQG | LPGPAGPPGE | AGKPGEQGVP |
| 1210 | 1220 | 1230 | 1240 | 1250 | 1260 |
| GDLGAPGPSG | ARGERGFPGE | RGVQGPPGPA | GPRGANGAPG | NDGAKGDAGA | PGAPGSQGAP |
| 1270 | 1280 | 1290 | 1300 | 1310 | 1320 |
| GLQGMPGERG | AAGLPGPKGD | RGDAGPKGAD | GSPGKDGVRG | LTGPIGPPGP | AGAPGDKGES |
| 1330 | 1340 | 1350 | 1360 | 1370 | 1380 |
| GPSGPAGPTG | ARGAPGDRGE | PGPPGPAGFA | GPPGADGQPG | AKGEPGDAGA | KGDAGPPGPA |
| 1390 | 1400 | 1410 | 1420 | 1430 | 1440 |
| GPAGPPGPIG | NVGAPGAKGA | RGSAGPPGAT | GFPGAAGRVG | PPGPSGNAGP | PGPPGPAGKE |
| 1450 | 1460 | 1470 | 1480 | 1490 | 1500 |
| GGKGPRGETG | PAGRPGEVGP | PGPPGPAGEK | GSPGADGPAG | APGTPGPQGI | AGQRGVVGLP |
| 1510 | 1520 | 1530 | 1540 | 1550 | 1560 |
| GQRGERGFPG | LPGPSGEPGK | QGPSGASGER | GPPGPMGPPG | LAGPPGESGR | EGAPGAEGSP |
| 1570 | 1580 | 1590 | 1600 | 1610 | 1620 |
| GRDGSPGAKG | DRGETGPAGP | PGAPGAPGAP | GPVGPAGKSG | DRGETGPAGP | AGPVGPVGAR |
| 1630 | 1640 | 1650 | 1660 | 1670 | 1680 |
| GPAGPQGPRG | DKGETGEQGD | RGIKGHRGFS | GLQGPPGPPG | SPGEQGPSGA | SGPAGPRGPP |
| 1690 | 1700 | 1710 | 1720 | 1730 | 1740 |
| GSAGAPGKDG | LNGLPGPIGP | PGPRGRTGDA | GPVGPPGPPG | PPGPPGPPSA | GFDFSFLPQP |
| 1750 | 1760 | 1770 | 1780 | 1790 | 1800 |
| PQEKAHDGGR | YYRAGSGECE | CSLLICEDVE | ENPCPQYDGK | GVGLGPGPMG | LMGPRGPPGA |
| 1810 | 1820 | 1830 | 1840 | 1850 | 1860 |
| AGAPGPQGFQ | GPAGEPGEPG | QTGPAGARGP | AGPPGKAGED | GHPGKPGRPG | ERGVVGPQGA |
| 1870 | 1880 | 1890 | 1900 | 1910 | 1920 |
| RGFPGTPGLP | GFKGIRGHNG | LDGLKGQPGA | PGVKGEPGAP | GENGTPGQTG | ARGLPGERGR |
| 1930 | 1940 | 1950 | 1960 | 1970 | 1980 |
| VGAPGPAGAR | GSDGSVGPVG | PAGPIGSAGP | PGFPGAPGPK | GEIGAVGNAG | PAGPAGPRGE |
| 1990 | 2000 | 2010 | 2020 | 2030 | 2040 |
| VGLPGLSGPV | GPPGNPGANG | LTGAKGAAGL | PGVAGAPGLP | GPRGIPGPVG | AAGATGARGL |
| 2050 | 2060 | 2070 | 2080 | 2090 | 2100 |
| VGEPGPAGSK | GESGNKGEPG | SAGPQGPPGP | SGEEGKRGPN | GEAGSAGPPG | PPGLRGSPGS |
| 2110 | 2120 | 2130 | 2140 | 2150 | 2160 |
| RGLPGADGRA | GVMGPPGSRG | ASGPAGVRGP | NGDAGRPGEP | GLMGPRGLPG | SPGNIGPAGK |
| 2170 | 2180 | 2190 | 2200 | 2210 | 2220 |
| EGPVGLPGID | GRPGPIGPAG | ARGEPGNIGF | PGPKGPTGDP | GKNGDKGHAG | LAGARGAPGP |
| 2230 | 2240 | 2250 | 2260 | 2270 | 2280 |
| DGNNGAQGPP | GPQGVQGGKG | EQGPPGPPGF | QGLPGPSGPA | GEVGKPGERG | LHGEFGLPGP |
| 2290 | 2300 | 2310 | 2320 | 2330 | 2340 |
| AGPRGERGPP | GESGAAGPTG | PIGSRGPSGP | PGPDGNKGEP | GVVGAVGTAG | PSGPSGLPGE |
| 2350 | 2360 | 2370 | 2380 | 2390 | 2400 |
| RGAAGIPGGK | GEKGEPGLRG | EIGNPGRDGA | RGAPGAVGAP | GPAGATGDRG | EAGAAGPAGP |
| 2410 | 2420 | 2430 | 2440 | 2450 | 2460 |
| AGPRGSPGER | GEVGPAGPNG | FAGPAGAAGQ | PGAKGERGAK | GPKGENGVVG | PTGPVGAAGP |
| 2470 | 2480 | 2490 | 2500 | 2510 | 2520 |
| AGPNGPPGPA | GSRGDGGPPG | MTGFPGAAGR | TGPPGPSGIS | GPPGPPGPAG | KEGLRGPRGD |
| 2530 | 2540 | 2550 | 2560 | 2570 | 2580 |
| QGPVGRTGEV | GAVGPPGFAG | EKGPSGEAGT | AGPPGTPGPQ | GLLGAPGILG | LPGSRGERGL |
| 2590 | 2600 | 2610 | 2620 | 2630 | 2640 |
| PGVAGAVGEP | GPLGIAGPPG | ARGPPGAVGS | PGVNGAPGEA | GRDGNPGNDG | PPGRDGQPGH |
| 2650 | 2660 | 2670 | 2680 | 2690 | 2700 |
| KGERGYPGNI | GPVGAAGAPG | PHGPVGPAGK | HGNRGETGPS | GPVGPAGAVG | PRGPSGPQGI |
| 2710 | 2720 | 2730 | 2740 | 2750 | 2760 |
| RGDKGEPGEK | GPRGLPGLKG | HNGLQGLPGI | AGHHGDQGAP | GSVGPAGPRG | PAGPSGPAGK |
| 2770 | 2780 | 2790 | 2800 | 2810 | |
| DGRTGHPGTV | GPAGIRGPQG | HQGPAGPPGP | PGPPGPPGVS | GGGYDEGYDG | DFYRA |

```
SEQ NO 20: Nucleotide Sequence of FIB3COL1, Codon Optimized for
Nicotiana Benthamiana Chloroplast Expression
      1 ATGGCTACTA CTTTGATTTC AAAGTTAACC CTTTCTAGTG CTTTCCTCGG CCAACAGTTT
    61 TCTTCTAGGG GTAATTCTAT GAGATCTGCA CCTGCAGGAT TGTTTCTTCG TGGACCAAGA
  121 GCTAGAGCAG GATCGGGTCA ACAAGGACCA GGACAACAGG GACCAGGACA ACAGGGTCCA
  181 GGTCAACAAG GACCATATGG TCCTGGAGCA TCAGCAGCTG CTGCTGCAGC TGGTGGATAC
  241 GGTCCAGGAA GCGGTCAACA AGGTCCATCC CAACAAGGTC CTGGTCAACA AGGACCAGGA
  301 GGGCAAGGTC CTTACGGACC TGGTGCTAGT GCAGCTGCTG CAGCTGCTGG AGGTTACGGA
  361 CCAGGTTCTG GTCAACAAGG ACCAGGAGGA CAAGGTCCAT ACGGACCAGG ATCTTCTGCT
  421 GCAGCTGCTG CTGCAGGAGG AAATGGTCCT GGATCTGGAC AACAAGGAGC AGGTCAACAA
  481 GGTCCTGGCC AACAAGGTCC AGGTGCTTCT GCTGCTGCTG CTGCAGCAGG TGGTTATGGT
  541 CCCGGATCAG GACAACAAGG TCCTGGACAA CAAGGTCCTG GAGGACAAGG ACCTTATGGT
  601 CCTGGTGCTA GTGCTGCTGC TGCTGCTGCT GGAGGATATG GTCCAGGAAG CGGACAAGGA
  661 CCAGGACAGC AAGGGCCTGG AGGTCAGGGT CCATATGGTC CTGGAGCTTC TGCAGCTGCT
  721 GCTGCTGCTG GTGGATATGG ACCAGGTTCT GGACAACAGG GTCCTGGTCA ACAAGGACCA
  781 GGACAGCAGG GACCAGGAGG TCAAGGTCCA TATGGACCTG GAGCATCAGC AGCTGCAGCA
  841 GCTGCAGGTG GCTATGGTCC TGGATATGGT CAACAGGGAC CTGGACAGCA GGGTCCTGGA
  901 GGTCAAGGTC CTTATGGTCC TGGAGCTTCA GCTGCTTCTG CAGCTTCCGG TGGATATGGA
  961 CCTGGATCTG GTCAGCAAGG CCCTGGTCAA CAAGGTCCAG GAGGTCAAGG ACCTTATGGG
1021 CCTGGAGCTT CTGCTGCTGC AGCTGCAGCT GGAGGATACG GACCTGGATC TGGTCAGCAA
1081 GGACCAGGTC AACAGGGTCC AGGTCAACAA GGACCAGGTC AACAAGGTCC AGGAGGGCAG
1141 GGACCATATG GACCTGGAGC TTCAGCAGCA GCTGCTGCTG CTGGTGGATA CGGTCCAGGT
1201 TCAGGACAAC AGGGCCCTGG ACAACAAGGA CCTGGCCAAC AAGGACCTGG TCAACAAGGT
1261 CCTGGTCAAC AAGGACCTGG TCAACAAGGA CCAGGACAAC AAGGACCAGG TCAACAGGGA
1321 CCAGGTCAAC AAGGTCCTGG AGGTCAGGGT GCTTATGGTC CAGGTGCTTC CGCTGCTGCT
1381 GGTGCTGCAG GTGGTTACGG ACCTGGATCT GGACAGCAAG GACCAGGTCA ACAAGGACCT
1441 GGACAACAAG GTCCAGGACA ACAAGGACCT GGACAACAAG GTCCAGGTCA ACAAGGTCCT
1501 GGTCAGCAGG GTCCAGGACA ACAAGGTCCT TATGGACCAG GGGCTAGCGC TGCTGCAGCA
1561 GCAGCAGGTG GATATGGACC AGGTAGTGGT CAACAAGGTC CTGGACAGCA AGGTCCTGGT
1621 CAACAAGGTC CTGGAGGTCA AGGACCCTAC GGTCCAGGTG CTGCTTCAGC AGCTGTGTCT
1681 GTTGGTGGAT ATGGACCACA GTCTTCTTCA GTCCCAGTTG CATCTGCAGT TGCATCTAGA
1741 CTTTCATCTC CAGCTGCTTC ATCTAGAGTT TCTTCTGCTG TTTCTTCTCT TGTGTCATCT
1801 GGTCCAACTA AACATGCTGC ACTTTCTAAC ACAATTAGTT CAGTTGTTTC TCAAGTTTCT
1861 GCATCTAACC CAGGACTTTC TGGTTGCGAT GTTCTTGTGC AAGCTCTTCT GGAAGTTGTT
1921 AGTGCTTTGG TTTCCATTTT GGGTTCTAGC TCTATTGGAC AGATCAATTA TGGTGCTTCA
1981 GCACAATACA CTCAAATGGT TGGACAAAGC GTTGCTCAGG CTCTTGCTGG AAGCGGAGAA
2041 GGAAGAGGTA GTCTGCTTAC ATGTGAAGAT GTTGAAGAAA ATCCTGGTCC ACAACTTTCA
2101 TATGGTTATG ATGAGAAATC AACAGGTGGT ATTTCTGTGC CAGGACCTAT GGGTCCTTCA
2161 GGCCCTAGAG GATTGCCAGG TCCACCTGGT GCTCCTGGTC CTCAAGGATT CCAAGGACCA
2221 CCAGGTGAGC CAGGTGAACC TGGAGCTAGT GGACCAATGG GTCCTAGAGG TCCACCTGGT
2281 CCTCCTGGTA AAAATGGTGA TGATGGAGAG GCAGGAAAGC CTGGAAGACC TGGAGAAAGA
2341 GGACCACCTG GACCTCAAGG AGCTCGGGGA CTTCCAGGTA CAGCTGGATT GCCAGGTATG
2401 AAGGGACACA GAGGATTCAG TGGCTTGGAT GGAGCTAAGG GAGATGCTGG TCCAGCTGGA
2461 CCTAAAGGAG AGCCAGGTTC TCCAGGAGAA AACGGAGCTC CAGGACAAAT GGGACCTAGA
2521 GGTCTTCCTG GTGAAAGGGG TAGGCCAGGA GCCCCTGGAC CTGCTGGTGC TAGAGGTAAC
2581 GATGGAGCTA CTGGTGCTGC TGGACCACCA GGACCTACTG GTCCTGCAGG TCCACCAGGT
2641 TTTCCAGGTG CAGTTGGAGC AAAGGGTGAG GCTGGTCCAC AAGGACCTAG AGGTTCAGAA
2701 GGACCACAAG GTGTTAGAGG TGAACCAGGT CCACCGGGAC CAGCAGGAGC CGCTGGCCCC
2761 GCTGGTAATC CTGGTGCTGA TGGTCAACCA GGTGCTAAGG GAGCTAACGG TGCTCCAGGG
2821 ATTGCTGGTG CTCCAGGATT CCCTGGAGCT AGAGGACCTT CAGGTCCACA AGGTCCTGGT
2881 GGACCACCTG GACCAAAAGG AAATAGCGGA GAGCCAGGTG CACCTGGCTC AAAGGGAGAT
2941 ACTGGAGCAA AGGGAGAGCC TGGACCTGTT GGTGTTCAAG GTCCTCCTGG ACCTGCTGGT
3001 GAGGAGGGAA AGAGAGGTGC AAGAGGTGAG CCTGGTCCTA CAGGACTCCC TGGTCCTCCT
3061 GGTGAAAGGG GAGGACCTGG ATCTAGGGGT TTTCCAGGTG CTGATGGAGT TGCTGGACCT
3121 AAAGGACCAG CTGGAGAAAG GGGATCTCCA GGTCCAGCTG GGCCAAAGGG TTCTCCTGGA
3181 GAGGCAGGAA GACCAGGTGA AGCTGGATTG CCAGGTGCCA AGGGACTTAC AGGATCTCCT
3241 GGGTCACCAG GACCAGATGG AAAGACTGGT CCTCCTGGAC CAGCTGGACA AGATGGAAGA
3301 CCTGGACCAC CTGGACCACC TGGAGCAAGG GGTCAAGCTG GTGTTATGGG TTTTCCAGGT
3361 CCAAAAGGTG CAGCAGGCGA GCCAGGAAAG GCTGGTGAAA GGGGTGTTCC AGGTCCACCT
3421 GGAGCAGTTG GTCCAGCTGG AAAGGATGGA GAGGCTGGCG CTCAAGGTCC TCCTGGTCCT
3481 GCTGGGCCAG CAGGTGAAAG AGGAGAACAA GGACCTGCTG GGTCTCCTGG TTTTCAAGGA
3541 CTTCCTGGAC CAGCTGGTCC TCCAGGTGAA GCAGGCAAGC CAGGAGAGCA AGGTGTTCCT
3601 GGAGATCTTG GTGCCCCAGG TCCTTCTGGT GCAAGAGGAG AGCGTGGATT CCCTGGAGAA
3661 AGAGGTGTGC AAGGTCCTCC AGGTCCAGCT GGTCCACGTG GAGCTAACGG AGCTCCTGGT
3721 AACGATGGAG CTAAAGGAGA TGCTGGTGCC CCAGGCGCAC CTGGTTCACA AGGTGCTCCT
3781 GGATTGCAAG GTATGCCTGG CGAAAGAGGT GCTGCTGGAC TTCCTGGACC TAAGGGTGAC
3841 AGAGGTGATG CTGGACCAAA AGGAGCTGAT GGATCACCTG GTAAAGATGG AGTGAGAGGT
3901 TTAACCGGTC CAATTGGACC ACCAGGTCCC GCTGGAGCTC CAGGAGATAA AGGAGAAAGT
3961 GGACCATCAG GTCCTGCCGG TCCCACTGGT GCTAGAGGTG CACCTGGTGA TAGAGGTGAA
4021 CCTGGTCCAC CAGGGCCTGC TGGATTTGCT GGTCCACCAG GAGCAGATGG ACAACCAGGA
4081 GCAAAAGGTG AGCCTGGAGA TGCTGGAGCT AAAGGAGATG CAGGTCCTCC TGGACCAGCT
4141 GGACCTGCTG GACCACCTGG ACCAATTGGA AATGTTGGTG CTCCAGGAGC TAAAGGGGCA
4201 AGAGGATCTG CTGGTCCTCC TGGAGCAACT GGGTTCCCTG GAGCAGCAGG AAGAGTTGGT
4261 CCTCCTGGAC CTTCTGGAAA CGCTGGACCT CCTGGTCCAC CAGGACCTGC TGGAAAGGAA
4321 GGAGGAAAGG GTCCAAGAGG CGAAACTGGA CCAGCAGGTA GACCAGGAGA GGTTGGACCA
4381 CCTGGACCAC CTGGTCCCGC TGGAGAGAAA GGATCTCCTG GAGCTGATGG ACCAGCAGGT
4441 GCTCCAGGCA CTCCAGGCCC ACAAGGAATT GCTGGTCAAA GGGGAGTIGT TGGATTGCCT
4501 GGGCAAAGAG GAGAGAGGGG ATTTCCTGGT CTTCCTGGTC CATCAGGTGA ACCTGGAAAA
4561 CAAGGTCCAT CTGGAGCTAG TGGTGAGAGG GGCCCTCCAG GACCAATGGG CCCACCTGGA
4621 CTTGCTGGAC CTCCTGGAGA GTCCGGTAGA GAAGGGGCTC CAGGTGCTGA AGGATCACCA
4681 GGAAGGGATG GATCTCCTGG AGCCAAGGGG GATAGAGGAG AAACAGGTCC AGCAGGGCCT
4741 CCTGGTGCAC CAGGTGCACC TGGTGCTCCT GGTCCAGTTG GACCTGCAGG TAAATCTGGT
4801 GATCGTGGAG AAACTGGTCC AGCTGGACCT GCTGGACCTG TTGGACCAGT GGGTGCTCGT
4861 GGACCTGCTG GTCCACAGGG ACCAAGAGGA GATAAAGGTG AGACTGGTGA GCAAGGTGAT
4921 AGAGGAATTA AAGGACATAG GGGTTTTTCT GGCTTACAGG GTCCTCCAGG TCCACCAGGA
4981 TCTCCAGGAG AACAAGGTCC ATCAGGAGCT AGTGGACCAG CAGGGCCAAG GGGACCTCCT
5041 GGTTCTGCTG GTGCACCAGG TAAAGATGGG CTTAACGGAT TGCCTGGACC TATAGGTCCT
5101 CCAGGTCCAA GAGGAAGAAC TGGTGATGCT GGTCCTGTTG GACCACCAGG TCCACCTGGA
5161 CCACCAGGGC CACCTGGACC TCCATCTGCA GGATTTGATT TTTCTTTCCT TCCACAACCA
5221 CCACAAGAAA AGGCTCACGA TGGTGGAAGG TATTATAGGG CAGGCTCTGG TGAAGGGCGT
5281 GGAAGTCTTC TTACATGTGA GGATGTTGAA GAAAATCCAG GACCACAATA TGATGGAAAG
5341 GGTGTTGGAT TGGGTCCAGG TCCAATGGGA TTGATGGGCC CTAGAGGTCC TCCTGGAGCT
5401 GCTGGAGCTC CTGGACCACA AGGATTCCAG GGCCCAGCTG GTGAACCTGG AGAACCGGGA
5461 CAAACAGGAC CAGCTGGTGC TAGAGGTCCA GCTGGTCCTC CAGGAAAAGC TGGAGAAGAT
5521 GGCCATCCTG GTAAACCAGG TAGGCCAGGA GAAAGAGGTG TTGTGGGTCC ACAGGGAGCT
5581 AGGGGATTTC CTGGTACTCC TGGGTTGCCT GGATTCAAGG GAATTAGGGG TCATAATGGT
5641 CTTGATGGTC TTAAAGGACA ACCAGGAGCT CCTGGTGTTA AAGGAGAACC TGGAGCACCT
5701 GGTGAAAATG GTACTCCAGG TCAAACAGGT GCAAGAGGAT TGCCAGGAGA AAGGGGTAGA
5761 GTGGGAGCAC CAGGTCCTGC TGGAGCTAGA GGTTCAGATG GAAGTGTGGG ACCTGTGGGA
5821 CCTGCAGGAC CAATTGGATC AGCTGGTCCA CCTGGATTTC CAGGTGCCCC AGGTCCAAAG
5881 GGAGAAATTG GAGCTGTTGG AAATGCGGGC CCAGCAGGCC CAGCTGGACC TAGAGGTGAG
5941 GTTGGTCTAC CAGGTCTGTC AGGACCAGTG GGCCCTCCAG GAAATCCTGG TGCAAATGGG
6001 CTTACAGGAG CTAAGGGAGC AGCTGGATTG CCTGGTGTTG CTGGGGCACC AGGTCTTCCT
6061 GGTCCAAGAG GTATTCCAGG ACCAGTAGGT GCTGCAGGAG CAACTGGAGC TAGAGGTTTG
6121 GTTGGTGAAC CAGGACCAGC AGGCTCCAAG GGTGAATCTG GTAATAAGGG AGAACCTGGT
6181 TCTGCTGGAC CACAAGGACC ACCAGGACCA TCAGGAGAAG AAGGTAAGAG GGGTCCTAAC
6241 GGAGAGGCCG GTTCTGCAGG TCCACCTGGA CCACCTGGAC TTAGAGGATC TCCAGGGTCT
6301 AGAGGTTTAC CTGGTGCTGA TGGTAGAGCT GGAGTGATGG GTCCTCCAGG TTCAAGAGGA
6361 GCATCTGGCC CAGCAGGAGT TAGGGGACCA AATGGTGATG CTGGGAGACC AGGTGAACCA
6421 GGTCTTATGG GTCCTAGAGG ATTGCCAGGT TCACCAGGAA ATATTGGTCC AGCTGGAAAA
6481 GAAGGACCAG TTGGACTTCC TGGAATTGAT GGTAGACCAG GTCCTATTGG TCCTGCTGGT
6541 GCTAGAGGTG AGCCAGGTAA TATCGGTTTT CCAGGACCAA AGGGACCAAC TGGTGATCCA
6601 GGCAAAAATG GTGATAAGGG ACATGCTGGA CTCGCAGGAG CTAGAGGCGC TCCAGGACCT
6661 GATGGAAATA ATGGTGCCCA GGGACCTCCA GGACCACAAG GTGTTCAAGG AGGAAAGGGT
6721 GAGCAAGGAC CTCCAGGACC TCCAGGTTTT CAGGGACTTC CAGGACCATC TGGACCAGCA
6781 GGTGAGGTTG GTAAGCCAGG AGAAAGGGGT TTACATGGTG AATTCGGTCT GCCAGGACCA
6841 GCTGGACCAA GGGGTGAAAG GGGTCCACCA GGAGAGTCAG GTGCTGCTGG ACCAACAGGA
6901 CCAATTGGTT CAAGAGGTCC ATCTGGACCT CCAGGTCCTG ATGGAAACAA AGGTGAACCA
6961 GGAGTTGTAG GTGCTGTTGG AACTGCTGGT CCTTCAGGCC CAAGCGGACT TCCAGGTGAA
7021 AGGGGTGCTG CTGGTATTCC TGGAGGTAAG GGTGAAAAAG GGGAGCCTGG TCTTAGAGGT
7081 GAGATTGGTA ATCCAGGAAG AGATGGGGCT AGAGGTGCAC CAGGAGCCGT TGGTGCTCCT
7141 GGTCCTGCTG GAGCTACAGG AGATAGAGGA GAGGCAGGAG CTGCTGGTCC TGCTGGACCA
7201 GCTGGCCCAA GAGGTAGCCC AGGAGAAAGA GGTGAAGTTG GTCCAGCTGG TCCTAATGGA
7261 TTTGCTGGAC CTGCTGGTGC TGCTGGTCAG CCTGGAGCTA AAGGGGAAAG AGGAGCCAAA
7321 GGACCTAAAG GAGAAAATGG AGTTGTTGGG CCTACAGGAC CAGTAGGAGC AGCAGGACCT
7381 GCTGGTCCAA ATGGACCACC AGGACCAGCA GGATCCAGAG GAGATGGTGG TCCACCAGGA
7441 ATGACAGGTT TTCCTGGTGC TGCTGGAAGA ACAGGACCAC CAGGTCCTTC AGGTATTTCT
7501 GGTCCTCCAG GTCCTCCAGG ACCAGCTGGA AAGGAGGGTT TGAGAGGACC TAGAGGTGAT
7561 CAAGGACCTG TGGGAAGAAC AGGAGAAGTT GGAGCAGTTG GACCACCAGG TTTCGCTGGA
7621 GAAAAGGGAC CATCTGGCGA AGCTGGAACT GCTGGACCAC CAGGTACCCC TGGACCTCAG
7681 GGACTTCTTG GAGCTCCTGG AATTTTGGGA CTTCCCGGAT CTAGAGGAGA GAGGGGATTG
7741 CCAGGCGTTG CTGGAGCTGT GGGTGAGCCA GGTCCTCTCG GAATTGCTGG ACCACCTGGT
7801 GCAAGGGGTC CACCAGGTGC CGTTGGGTCC CCAGGTGTTA ATGGTGCTCC AGGTGAGGCT
7861 GGTAGGGATG GAAATCCAGG TAATGATGGA CCACCTGGTA GGGATGGACA GCCTGGCCAT
7921 AAGGGTGAGC GTGGATATCC AGGTAATATT GGACCAGTTG GTGCAGCAGG GGCACCAGGA
7981 CCACACGGAC CTGTTGGTCC AGCTGGTAAG CACGGCAATA GGGGAGAGAC TGGTCCTTCA
8041 GGACCTGTGG GTCCGGCTGG AGCAGTTGGA CCTAGAGGTC CATCAGGACC ACAAGGAATT
8101 AGAGGTGATA AGGGAGAACC CGGGGAGAAA GGACCAAGAG GATTACCTGG ATTAAAGGGT
8161 CACAATGGAT TACAAGGATT GCCTGGAATT GCTGGACATC ACGGAGATCA AGGAGCACCA
8221 GGATCAGTTG GACCGGCTGG ACCAAGAGGA CCTGCAGGAC CTTCTGGACC TGCTGGTAAA
8281 GATGGAAGAA CTGGACATCC TGGTACAGTT GGACCTGCTG GAATTAGAGG TCCACAAGGT
8341 CATCAAGGGC CTGCCGGTCC TCCAGGACCA CCAGGACCAC CAGGGCCTCC AGGAGTTTCT
8401 GGCGGTGGAT ATGATTTTGG TTATGATGGA GATTTTTACC GTGCTTGA
```

What is claimed:

1. A method of producing fusion proteins of a non-human scleroprotein with a human collagen , wherein said fusion proteins are capable of forming hydroxylated triple helix fibers, hereinafter also referred to as a self-fibrillating heterotrimeric collagen comprising fusion protein, in a plant or an isolated plant cell comprising:
(a) Targeting to and accumulating in a chloroplast of the plant or the isolated plant cell a nucleotide sequence encoding a non-human scleroprotein, a nucleotide sequence encoding a human Collagen Type-I Alpha-I chain, a nucleotide sequence encoding a human Collagen Type-I Alpha-II chain, including a signal peptide sequence for targeting to a chloroplast as set forth by SEQ ID 16, all of which said sequences are devoid of an ER retention sequence,
(b) Targeting to and accumulating in a chloroplast of the plant or the isolated plant cell a nucleotide sequence encoding an exogenous non-human chimeric Prolyl 4 Hydroxylase (P4H) capable of specifically hydroxylating the Y position of Gly-X-Y triplets of said Collagen Type-I Alpha-I chain and said Collagen Type-I Alpha-II chain, and
(c) Co-expressing the genes of (a) and (b) in said chloroplast of the plant or the isolated plant cell, thereby obtaining fusion proteins of the non-human scleroprotein with a human collagen

2. The method of claim 1, wherein co-expressing the genes of (a) and (b) is done in two separate vectors and by means of an A2-enabled tricistronic expression vector that enables the induction of ribosomal skipping during translation of a protein in a cell, thereby making it possible to express the genes of (a) in parallel with the genes of (b) in one single plant; in particular by means of an A2-enabled tricistronic expression vector having an A2 sequence as set forth in SEQ ID 18 and 20.

3. The method of claim 1, wherein said exogenous non-human chimeric P4H comprises A) a non-human P4H alpha subunit sequence as set forth by SEQ ID's 11 and 18, and B) a non-human P4H beta subunit sequence as set forth by SEQ ID's 12 and 18, an exogenous human Lysine Hydroxylase 3 (LH3) sequence capable of specifically hydroxylating collagen lysines into 1,2-glucosylgalactosyl-5-hydroxylysines of said Collagen Type-I Alpha-I chain and said Collagen Type-I Alpha-II chain.

4. The method of claim 3, wherein said exogenous human LH3 is as set forth by SEQ ID's 14 and 18, including a signal peptide sequence for targeting to a chloroplast as set forth by SEQ ID 18, all of which said sequences are devoid of an ER retention sequence.

5. The method according to any one of the previous claims, wherein the method comprises avoiding the co-expression of a C-terminus and/or an N-terminus Collagen propeptide which are necessary for the assembly of collagen molecules into fibrils and thus enabling the formation of a triple-helical fibril structure.

6. The method according to any one of the previous claims, wherein the non-human scleroprotein is selected from Spidroin-I or Fibroin-III.

7. The method according to claim 6, wherein the Spidroin-I is encoded by a nucleotide sequence as set forth by SEQ ID's 6 and 16.

8. The method according to claim 6, wherein the Fibroin-III is encoded by a nucleotide sequence as set forth by SEQ ID's 8 and 20.

9. The method of claim 1, wherein said plant is a Nicotiana benthamiana or Nicotiana tabacum plant, and

10. The method of claim 1 further comprises filtrating and/or purifying the extracted fusion proteins of the non-human scleroprotein with a human collagen.

11. The method of claim 10, wherein said filtrating and/or purifying comprises a chromatography process.

12. The method of claim 1, wherein said plant is transiently transformed.

13. The method of claim 12 comprising introducing the nucleotide sequences, using the viral vector of any of the claims (a) to (c), into at least one Agrobacterium tumefaciens strain.

14. The fusion proteins of a non-human scleroprotein with a human collagen obtained using a method according to any one of the previous claims.

15. Use of the fusion proteins according to claim 14, for producing nano fibers.
